SEQUENCE LISTING (209 sequences)

SEQ ID NO: 1 - protein, 189 aa, Homo sapiens
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 2 - protein, 10 aa, Homo sapiens
VVVGADGVGK

SEQ ID NO: 3 - protein, 7 aa, Mus musculus
TIYSNPF

SEQ ID NO: 4 - protein, 7 aa, Mus musculus
SFTDNKR

SEQ ID NO: 5 - protein, 13 aa, Mus musculus
CALRGNAGAKLTF

SEQ ID NO: 6 - protein, 5 aa, Mus musculus
LGHDT

SEQ ID NO: 7 - protein, 6 aa, Mus musculus
YNNKQL

SEQ ID NO: 8 - protein, 15 aa, Mus musculus
CASSSRDWSAETLYF

SEQ ID NO: 9 - protein, 135 aa, Mus musculus
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAGAKLTFGGGTRLTVRPD

SEQ ID NO: 10 - protein, 134 aa, Mus musculus
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSAETLYFGSGTRLTVLE

SEQ ID NO: 11 - protein, 271 aa, Mus musculus
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAGAKLTFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 12 - protein, 306 aa, Mus musculus
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 13 - protein, 136 aa, Mus musculus
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 14 - protein, 172 aa, Mus musculus
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 15 - DNA, 403 bp, synthetic construct
  misc_feature 1..403: note = Synthetic
  source 1..403: mol_type = other DNA
atgcgtcctgtcacctgctcagttcttgtgctcctcctaatgctcaggaggagcaatggcgatggagactccgtgacccagacagaaggcctggtcactctcacagaagggttgcctgtgatgctgaactgcacctatcagactatttactcaaatcctttccttttctggtatgtgcaacatctcaatgaatcccctcggctactcctgaagagcttcacagacaacaagaggaccgagcaccaagggttccacgccactctccataagagcagcagctccttccatctgcagaagtcctcagcgcagctgtcagactctgccctgtactactgtgctctgagggggaatgcaggtgccaagctcacattcggagggggaacaaggttaacggtcagacccg

SEQ ID NO: 16 - DNA, 400 bp, synthetic construct
  misc_feature 1..400: note = Synthetic
  source 1..400: mol_type = other DNA
atgggctgtaggctcctaagctgtgtggccttctgcctcttgggaataggccctttggagacggctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagctcccgggactggagtgcagaaacgctgtattttggctcaggaaccagactgactgttctcg

SEQ ID NO: 17 - DNA, 816 bp, synthetic construct
  misc_feature 1..816: note = Synthetic
  source 1..816: mol_type = other DNA
atgcgtcctgtcacctgctcagttcttgtgctcctcctaatgctcaggaggagcaatggcgatggagactccgtgacccagacagaaggcctggtcactctcacagaagggttgcctgtgatgctgaactgcacctatcagactatttactcaaatcctttccttttctggtatgtgcaacatctcaatgaatcccctcggctactcctgaagagcttcacagacaacaagaggaccgagcaccaagggttccacgccactctccataagagcagcagctccttccatctgcagaagtcctcagcgcagctgtcagactctgccctgtactactgtgctctgagggggaatgcaggtgccaagctcacattcggagggggaacaaggttaacggtcagacccgacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagttga

SEQ ID NO: 18 - DNA, 921 bp, synthetic construct
  misc_feature 1..921: note = Synthetic
  source 1..921: mol_type = other DNA
atgggctgtaggctcctaagctgtgtggccttctgcctcttgggaataggccctttggagacggctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagctcccgggactggagtgcagaaacgctgtattttggctcaggaaccagactgactgttctcgaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctga

SEQ ID NO: 19 - DNA, 413 bp, synthetic construct
  misc_feature 1..413: note = Synthetic
  source 1..413: mol_type = other DNA
acatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagttga

SEQ ID NO: 20 - DNA, 521 bp, synthetic construct
  misc_feature 1..521: note = Synthetic
  source 1..521: mol_type = other DNA
aggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctagtgctgatggccatggtcaagaaaaaaaattcctga

SEQ ID NO: 21 - DNA, 1815 bp, synthetic construct
  misc_feature 1..1815: note = Synthetic
  source 1..1815: mol_type = other DNA
atgcgtcctgtcacctgctcagttcttgtgctcctcctaatgctcaggaggagcaatggcgatggagactccgtgacccagacagaaggcctggtcactctcacagaagggttgcctgtgatgctgaactgcacctatcagactatttactcaaatcctttccttttctggtatgtgcaacatctcaatgaatcccctcggctactcctgaagagcttcacagacaacaagaggaccgagcaccaagggttccacgccactctccataagagcagcagctccttccatctgcagaagtcctcagcgcagctgtcagactctgccctgtactactgtgctctgagggggaatgcaggtgccaagctcacattcggagggggaacaaggttaacggtcagacccgacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagtcgggccaagcggtccggatccggagccaccaacttcagcctgctgaagcaggccggcgacgtggaggagaaccccggccccatgggctgtaggctcctaagctgtgtggccttctgcctcttgggaataggccctttggagacggctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagctcccgggactggagtgcagaaacgctgtattttggctcaggaaccagactgactgttctcgaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctga

SEQ ID NO: 22 - DNA, 21 bp, synthetic construct
  misc_feature 1..21: note = Synthetic
  source 1..21: mol_type = other DNA
actatttactcaaatcctttc

SEQ ID NO: 23 - DNA, 21 bp, synthetic construct
  misc_feature 1..21: note = Synthetic
  source 1..21: mol_type = other DNA
agcttcacagacaacaagagg

SEQ ID NO: 24 - DNA, 33 bp, synthetic construct
  misc_feature 1..33: note = Synthetic
  source 1..33: mol_type = other DNA
gctctgagggggaatgcaggtgccaagctcaca

SEQ ID NO: 25 - DNA, 15 bp, synthetic construct
  misc_feature 1..15: note = Synthetic
  source 1..15: mol_type = other DNA
ctgggccacgatact

SEQ ID NO: 26 - DNA, 18 bp, synthetic construct
  misc_feature 1..18: note = Synthetic
  source 1..18: mol_type = other DNA
tacaataataagcaactc

SEQ ID NO: 27 - DNA, 39 bp, synthetic construct
  misc_feature 1..39: note = Synthetic
  source 1..39: mol_type = other DNA
gccagcagctcccgggactggagtgcagaaacgctgtat

SEQ ID NO: 28 - protein, 27 aa, synthetic construct
  REGION 1..27: note = Synthetic
RAKRSGSGATNFSLLKQAGDVEENPGP

SEQ ID NO: 29 - protein, 189 aa, Homo sapiens [sequence omitted]

SEQ ID NO: 30 - protein, 10 aa, Homo sapiens
VVVGAGGVGK

SEQ ID NO: 31 - protein, 9 aa, Homo sapiens
VVGAGGVGK

SEQ ID NO: 32 - protein, 189 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 33 - protein, 10 aa, Homo sapiens
VVVGAVGVGK

SEQ ID NO: 34 - protein, 9 aa, Homo sapiens
VVGADGVGK

SEQ ID NO: 35 - protein, 9 aa, Homo sapiens
VVGAVGVGK

SEQ ID NO: 36 - protein, 9 aa, Homo sapiens
VVGACGVGK

SEQ ID NO: 37 - protein, 10 aa, Homo sapiens
VVVGACGVGK

SEQ ID NO: 38 - protein, 9 aa, Homo sapiens
VVGARGVGK

SEQ ID NO: 39 - protein, 10 aa, Homo sapiens
VVVGARGVGK

SEQ ID NO: 40 - protein, 16 aa, Mus musculus
CAMREDTGANTGKLTF

SEQ ID NO: 41 - protein, 15 aa, Mus musculus
CASSQDSLGRAEQFF

SEQ ID NO: 42 - protein, 15 aa, Mus musculus
CASSSDWGGAETLYF

SEQ ID NO: 43 - protein, 16 aa, Mus musculus
CASSSGLGSSAETLYF

SEQ ID NO: 44 - DNA, 81 bp, synthetic construct
  misc_feature 1..81: note = Synthetic
  source 1..81: mol_type = other DNA
cgggccaagcggtccggatccggagccaccaacttcagcctgctgaagcaggccggcgacgtggaggagaaccccggcccc

SEQ ID NO: 45 - protein, 604 aa, synthetic construct
  REGION 1..604: note = Synthetic
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAGAKLTFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 46 - protein, 13 aa, synthetic construct
  REGION 1..13: note = Synthetic
  VARIANT 2: note = X is Arg, Asn, Asp, Cys, Glu, Gln, Gly, His, Ile, Leu, Lys, Met, Phe, Pro, Ser, Thr, Trp, Tyr, or Val.
CXLRGNAGAKLTF

SEQ ID NO: 47 - protein, 13 aa, synthetic construct
  REGION 1..13: note = Synthetic
  VARIANT 3: note = X is Ala, Arg, Asn, Asp, Cys, Glu, Gln, Gly, His, Ile, Lys, Met, Phe, Pro, Ser, Thr, Trp, Tyr, or Val.
CAXRGNAGAKLTF

SEQ ID NO: 48 - protein, 13 aa, synthetic construct
  REGION 1..13: note = Synthetic
  VARIANT 4: note = X is Ala, Asn, Asp, Cys, Glu, Gln, Gly, His, Ile, Leu, Lys, Met, Phe, Pro, Ser, Thr, Trp, Tyr, or Val.
CALXGNAGAKLTF

SEQ ID NO: 49 - protein, 13 aa, synthetic construct
  REGION 1..13: note = Synthetic
  VARIANT 5: note = X Ala, Arg, Asn, Asp, Cys, Glu, Gln, His, Ile, Leu, Lys, Met, Phe, Pro, Ser, Thr, Trp, Tyr, or Val.
CALRXNAGAKLTF

SEQ ID NO: 50 - protein, 13 aa, synthetic construct
  REGION 1..13: note = Synthetic
  VARIANT 6: note = X is Ala, Arg, Asp, Cys, Glu, Gln, Gly, His, Ile, Leu, Lys, Met, Phe, Pro, Ser, Thr, Trp, Tyr, or Val.
CALRGXAGAKLTF

SEQ ID NO: 51 - protein, 13 aa, synthetic construct
  REGION 1..13: note = Synthetic
  VARIANT 7: note = X is Arg, Asn, Asp, Cys, Glu, Gln, Gly, His, Ile, Leu, Lys, Met, Phe, Pro, Ser, Thr, Trp, Tyr, or Val.
CALRGNXGAKLTF

SEQ ID NO: 52 - protein, 13 aa, synthetic construct
  REGION 1..13: note = Synthetic
  VARIANT 8: note = X is Ala, Arg, Asn, Asp, Cys, Glu, Gln, His, Ile, Leu, Lys, Met, Phe, Pro, Ser, Thr, Trp, Tyr, or Val.
CALRGNAXAKLTF

SEQ ID NO: 53 - protein, 13 aa, synthetic construct
  REGION 1..13: note = Synthetic
  VARIANT 9: note = X is Arg, Asn, Asp, Cys, Glu, Gln, Gly, His, Ile, Leu, Lys, Met, Phe, Pro, Ser, Thr, Trp, Tyr, or Val.
CALRGNAGXKLTF

SEQ ID NO: 54 - protein, 13 aa, synthetic construct
  REGION 1..13: note = Synthetic
  VARIANT 10: note = X is Ala, Arg, Asn, Asp, Cys, Glu, Gln, Gly, His, Ile, Leu, Met, Phe, Pro, Ser, Thr, Trp, Tyr, or Val.
CALRGNAGAXLTF

SEQ ID NO: 55 - protein, 13 aa, synthetic construct
  REGION 1..13: note = Synthetic
  VARIANT 11: note = X is Ala, Arg, Asn, Asp, Cys, Glu, Gln, Gly, His, Ile, Lys, Met, Phe, Pro, Ser, Thr, Trp, Tyr, or Val.
CALRGNAGAKXTF

SEQ ID NO: 56 - protein, 13 aa, synthetic construct
  REGION 1..13: note = Synthetic
  VARIANT 12: note = X is Ala, Arg, Asn, Asp, Cys, Glu, Gln, Gly, His, Ile, Leu, Lys, Met, Phe, Pro, Ser, Trp, Tyr, or Val.
CALRGNAGAKLXF

SEQ ID NO: 57 - protein, 15 aa, synthetic construct
  REGION 1..15: note = Synthetic
  VARIANT 2: note = X is Arg, Asn, Asp, Cys, Glu, Gln, Gly, His, Ile, Leu, Lys, Met, Phe, Pro, Ser, Thr, Trp, Tyr, or Val.
CXSSSRDWSAETLYF

SEQ ID NO: 58 - protein, 15 aa, synthetic construct
  REGION 1..15: note = Synthetic
  VARIANT 3: note = X is Ala, Arg, Asn, Asp, Cys, Glu, Gln, Gly, His, Ile, Leu, Lys, Met, Phe, Pro, Thr, Trp, Tyr, or Val.
CAXSSRDWSAETLYF

SEQ ID NO: 59 - protein, 15 aa, synthetic construct
  REGION 1..15: note = Synthetic
  VARIANT 4: note = X is Ala, Arg, Asn, Asp, Cys, Glu, Gln, Gly, His, Ile, Leu, Lys, Met, Phe, Pro, Thr, Trp, Tyr, or Val.
CASXSRDWSAETLYF

SEQ ID NO: 60 - protein, 15 aa, synthetic construct
  REGION 1..15: note = Synthetic
  VARIANT 5: note = X is Ala, Arg, Asn, Asp, Cys, Glu, Gln, Gly, His, Ile, Leu, Lys, Met, Phe, Pro, Thr, Trp, Tyr, or Val.
CASSXRDWSAETLYF

SEQ ID NO: 61 - protein, 15 aa, synthetic construct
  REGION 1..15: note = Synthetic
  VARIANT 6: note = X is Ala, Asn, Asp, Cys, Glu, Gln, Gly, His, Ile, Leu, Lys, Met, Phe, Pro, Ser, Thr, Trp, Tyr, or Val.
CASSSXDWSAETLYF

SEQ ID NO: 62 - protein, 15 aa, synthetic construct
  REGION 1..15: note = Synthetic
  VARIANT 7: note = X is Ala, Arg, Asn, Cys, Glu, Gln, Gly, His, Ile, Leu, Lys, Met, Phe, Pro, Ser, Thr, Trp, Tyr, or Val.
CASSSRXWSAETLYF

SEQ ID NO: 63 - protein, 15 aa, synthetic construct
  REGION 1..15: note = Synthetic
  VARIANT 8: note = X is Ala, Arg, Asn, Asp, Cys, Glu, Gln, Gly, His, Ile, Leu, Lys, Met, Phe, Pro, Ser, Thr, Tyr, or Val.
CASSSRDXSAETLYF

SEQ ID NO: 64 - protein, 15 aa, synthetic construct
  REGION 1..15: note = Synthetic
  VARIANT 9: note = X is Ala, Arg, Asn, Asp, Cys, Glu, Gln, Gly, His, Ile, Leu, Lys, Met, Phe, Pro, Thr, Trp, Tyr, or Val.
CASSSRDWXAETLYF

SEQ ID NO: 65 - protein, 15 aa, synthetic construct
  REGION 1..15: note = Synthetic
  VARIANT 10: note = X is Arg, Asn, Asp, Cys, Glu, Gln, Gly, His, Ile, Leu, Lys, Met, Phe, Pro, Ser, Thr, Trp, Tyr, or Val.
CASSSRDWSXETLYF

SEQ ID NO: 66 - protein, 15 aa, synthetic construct
  REGION 1..15: note = Synthetic
  VARIANT 11: note = X is Ala, Arg, Asn, Asp, Cys, Gln, Gly, His, Ile, Leu, Lys, Met, Phe, Pro, Ser, Thr, Trp, Tyr, or Val.
CASSSRDWSAXTLYF

SEQ ID NO: 67 - protein, 15 aa, synthetic construct
  REGION 1..15: note = Synthetic
  VARIANT 12: note = X is Ala, Arg, Asn, Asp, Cys, Glu, Gln, Gly, His, Ile, Leu, Lys, Met, Phe, Pro, Ser, Trp, Tyr, or Val.
CASSSRDWSAEXLYF

SEQ ID NO: 68 - protein, 15 aa, synthetic construct
  REGION 1..15: note = Synthetic
  VARIANT 13: note = X is Ala, Arg, Asn, Asp, Cys, Glu, Gln, Gly, His, Ile, Lys, Met, Phe, Pro, Ser, Thr, Trp, Tyr, or Val.
CASSSRDWSAETXYF

SEQ ID NO: 69 - protein, 15 aa, synthetic construct
  REGION 1..15: note = Synthetic
  VARIANT 14: note = X is Ala, Arg, Asn, Asp, Cys, Glu, Gln, Gly, His, Ile, Leu, Lys, Met, Phe, Pro, Ser, Thr, Trp, or Val.
CASSSRDWSAETLXF

SEQ ID NO: 70 - protein, 135 aa, synthetic construct
  REGION 1..135: note = Synthetic
  VARIANT 113: note = X is Arg, Asn, Asp, Cys, Glu, Gln, Gly, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCXLRGNAGAKLTFGGGTRLTVRPD

SEQ ID NO: 71 - protein, 135 aa, synthetic construct
  REGION 1..135: note = Synthetic
  VARIANT 114: note = X is Ala, Arg, Asn, Asp, Cys, Glu, Gln, Gly, His, Ile, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCAXRGNAGAKLTFGGGTRLTVRPD

SEQ ID NO: 72 - protein, 135 aa, synthetic construct
  REGION 1..135: note = Synthetic
  VARIANT 115: note = X is Ala, Asn, Asp, Cys, Glu, Gln, Gly, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALXGNAGAKLTFGGGTRLTVRPD

SEQ ID NO: 73 - protein, 135 aa, synthetic construct
  REGION 1..135: note = Synthetic
  VARIANT 116: note = X is Ala, Arg, Asn, Asp, Cys, Glu, Gln, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRXNAGAKLTFGGGTRLTVRPD

SEQ ID NO: 74 - protein, 135 aa, synthetic construct
  REGION 1..135: note = Synthetic
  VARIANT 117: note = X is Ala, Arg, Asp, Cys, Glu, Gln, Gly, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGXAGAKLTFGGGTRLTVRPD

SEQ ID NO: 75 - protein, 135 aa, synthetic construct
  REGION 1..135: note = Synthetic
  VARIANT 118: note = X is Arg, Asn, Asp, Cys, Glu, Gln, Gly, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNXGAKLTFGGGTRLTVRPD

SEQ ID NO: 76 - protein, 135 aa, synthetic construct
  REGION 1..135: note = Synthetic
  VARIANT 119: note = X is Ala, Arg, Asn, Asp, Cys, Glu, Gln, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAXAKLTFGGGTRLTVRPD

SEQ ID NO: 77 - protein, 135 aa, synthetic construct
  REGION 1..135: note = Synthetic
  VARIANT 120: note = X is Arg, Asn, Asp, Cys, Glu, Gln, Gly, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAGXKLTFGGGTRLTVRPD

SEQ ID NO: 78 - protein, 135 aa, synthetic construct
  REGION 1..135: note = Synthetic
  VARIANT 121: note = X is Ala, Arg, Asn, Asp, Cys, Glu, Gln, Gly, His, Ile, Leu, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAGAXLTFGGGTRLTVRPD

SEQ ID NO: 79 - protein, 135 aa, synthetic construct
  REGION 1..135: note = Synthetic
  VARIANT 122: note = X is Ala, Arg, Asn, Asp, Cys, Glu, Gln, Gly, His, Ile, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAGAKXTFGGGTRLTVRPD

SEQ ID NO: 80 - protein, 135 aa, synthetic construct
  REGION 1..135: note = Synthetic
  VARIANT 123: note = X is Ala, Arg, Asn, Asp, Cys, Glu, Gln, Gly, His, Ile, Leu, Lys, Met, Phe, Pro, Ser, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAGAKLXFGGGTRLTVRPD

SEQ ID NO: 81 - protein, 134 aa, synthetic construct
  REGION 1..134: note = Synthetic
  VARIANT 111: note = X is Arg, Asn, Asp, Cys, Glu, Gln, Gly, His, Ile, Leu, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCXSSSRDWSAETLYFGSGTRLTVLE

SEQ ID NO: 82 - protein, 134 aa, synthetic construct
  REGION 1..134: note = Synthetic
  VARIANT 112: note = X is Ala, Arg, Asn, Asp, Cys, Glu, Gln, Gly, His, Ile, Leu, Lys, Met, Phe, Pro, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCAXSSRDWSAETLYFGSGTRLTVLE

SEQ ID NO: 83 - protein, 134 aa, synthetic construct
  REGION 1..134: note = Synthetic
  VARIANT 113: note = X is Ala, Arg, Asn, Asp, Cys, Glu, Gln, Gly, His, Ile, Leu, Lys, Met, Phe, Pro, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASXSRDWSAETLYFGSGTRLTVLE

SEQ ID NO: 84 - protein, 134 aa, synthetic construct
  REGION 1..134: note = Synthetic
  VARIANT 114: note = X is Ala, Arg, Asn, Asp, Cys, Glu, Gln, Gly, His, Ile, Leu, Lys, Met, Phe, Pro, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSXRDWSAETLYFGSGTRLTVLE

SEQ ID NO: 85 - protein, 134 aa, synthetic construct
  REGION 1..134: note = Synthetic
  VARIANT 115: note = X is Ala, Asn, Asp, Cys, Glu, Gln, Gly, His, Ile, Leu, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSXDWSAETLYFGSGTRLTVLE

SEQ ID NO: 86 - protein, 134 aa, synthetic construct
  REGION 1..134: note = Synthetic
  VARIANT 116: note = X is Ala, Arg, Asn, Cys, Glu, Gln, Gly, His, Ile, Leu, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRXWSAETLYFGSGTRLTVLE

SEQ ID NO: 87 - protein, 134 aa, synthetic construct
  REGION 1..134: note = Synthetic
  VARIANT 117: note = X is Ala, Arg, Asn, Asp, Cys, Glu, Gln, Gly, His, Ile, Leu, Lys, Met, Phe, Pro, Ser, Thr, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDXSAETLYFGSGTRLTVLE

SEQ ID NO: 88 - protein, 134 aa, synthetic construct
  REGION 1..134: note = Synthetic
  VARIANT 118: note = X is Ala, Arg, Asn, Asp, Cys, Glu, Gln, Gly, His, Ile, Leu, Lys, Met, Phe, Pro, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWXAETLYFGSGTRLTVLE

SEQ ID NO: 89 - protein, 134 aa, synthetic construct
  REGION 1..134: note = Synthetic
  VARIANT 119: note = X is Arg, Asn, Asp, Cys, Glu, Gln, Gly, His, Ile, Leu, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSXETLYFGSGTRLTVLE

SEQ ID NO: 90 - protein, 134 aa, synthetic construct
  REGION 1..134: note = Synthetic
  VARIANT 120: note = X is Ala, Arg, Asn, Asp, Cys, Gln, Gly, His, Ile, Leu, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSAXTLYFGSGTRLTVLE

SEQ ID NO: 91 - protein, 134 aa, synthetic construct
  REGION 1..134: note = Synthetic
  VARIANT 121: note = X is Ala, Arg, Asn, Asp, Cys, Glu, Gln, Gly, His, Ile, Leu, Lys, Met, Phe, Pro, Ser, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSAEXLYFGSGTRLTVLE

SEQ ID NO: 92 - protein, 134 aa, synthetic construct
  REGION 1..134: note = Synthetic
  VARIANT 122: note = X is Ala, Arg, Asn, Asp, Cys, Glu, Gln, Gly, His, Ile, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSAETXYFGSGTRLTVLE

SEQ ID NO: 93 - protein, 134 aa, synthetic construct
  REGION 1..134: note = 'Synthetic
  VARIANT 123: note = X is Ala, Arg, Asn, Asp, Cys, Glu, Gln, Gly, His, Ile, Leu, Lys, Met, Phe, Pro, Ser, Thr, Trp, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSAETLXFGSGTRLTVLE

SEQ ID NO: 94 - protein, 271 aa, synthetic construct
  REGION 1..271: note = Synthetic
  VARIANT 113: note = X is Arg, Asn, Asp, Cys, Glu, Gln, Gly, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCXLRGNAGAKLTFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 95 - protein, 271 aa, synthetic construct
  REGION 1..271: note = Synthetic
  VARIANT 114: note = X is Ala, Arg, Asn, Asp, Cys, Glu, Gln, Gly, His, Ile, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCAXRGNAGAKLTFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 96 - protein, 271 aa, synthetic construct
  REGION 1..271: note = Synthetic
  VARIANT 115: note = X is Ala, Asn, Asp, Cys, Glu, Gln, Gly, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALXGNAGAKLTFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 97 - protein, 271 aa, synthetic construct
  REGION 1..271: note = Synthetic
  VARIANT 116: note = X is Ala, Arg, Asn, Asp, Cys, Glu, Gln, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRXNAGAKLTFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 98 - protein, 271 aa, synthetic construct
  REGION 1..271: note = Synthetic
  VARIANT 117: note = X is Ala, Arg, Asp, Cys, Glu, Gln, Gly, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGXAGAKLTFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 99 - protein, 271 aa, synthetic construct
  REGION 1..271: note = Synthetic
  VARIANT 118: note = X is Arg, Asn, Asp, Cys, Glu, Gln, Gly, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNXGAKLTFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 100 - protein, 271 aa, synthetic construct
  REGION 1..271: note = Synthetic
  VARIANT 119: note = X is Ala, Arg, Asn, Asp, Cys, Glu, Gln, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAXAKLTFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 101 - protein, 271 aa, synthetic construct
  REGION 1..271: note = Synthetic
  VARIANT 120: note = X is Arg, Asn, Asp, Cys, Glu, Gln, Gly, His, Ile, Leu, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAGXKLTFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 102 - protein, 271 aa, synthetic construct
  REGION 1..271: note = Synthetic
  VARIANT 121: note = X is Ala, Arg, Asn, Asp, Cys, Glu, Gln, Gly, His, Ile, Leu, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAGAXLTFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 103 - protein, 271 aa, synthetic construct
  REGION 1..271: note = Synthetic
  VARIANT 122: note = X is Ala, Arg, Asn, Asp, Cys, Glu, Gln, Gly, His, Ile, Lys, Met, Phe, Pro, Ser, Thr, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAGAKXTFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 104 - protein, 271 aa, synthetic construct
  REGION 1..271: note = Synthetic
  VARIANT 123: note = X is Ala, Arg, Asn, Asp, Cys, Glu, Gln, Gly, His, Ile, Leu, Lys, Met, Phe, Pro, Ser, Trp, Tyr, or Val.
MRPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCALRGNAGAKLXFGGGTRLTVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 105 - protein, 306 aa, synthetic construct
  REGION 1..306: note = Synthetic
  VARIANT 111: note = X is Arg, Asn, Asp, Cys, Glu, Gln, Gly, His, Ile, Leu, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCXSSSRDWSAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 106 - protein, 306 aa, synthetic construct
  REGION 1..306: note = Synthetic
  VARIANT 112: note = X is Ala, Arg, Asn, Asp, Cys, Glu, Gln, Gly, His, Ile, Leu, Lys, Met, Phe, Pro, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCAXSSRDWSAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 107 - protein, 306 aa, synthetic construct
  REGION 1..306: note = Synthetic
  VARIANT 113: note = X is Ala, Arg, Asn, Asp, Cys, Glu, Gln, Gly, His, Ile, Leu, Lys, Met, Phe, Pro, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASXSRDWSAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 108 - protein, 306 aa, synthetic construct
  REGION 1..306: note = Synthetic
  VARIANT 114: note = X is Ala, Arg, Asn, Asp, Cys, Glu, Gln, Gly, His, Ile, Leu, Lys, Met, Phe, Pro, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSXRDWSAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 109 - protein, 306 aa, synthetic construct
  REGION 1..306: note = Synthetic
  VARIANT 115: note = X is Ala, Asn, Asp, Cys, Glu, Gln, Gly, His, Ile, Leu, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSXDWSAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 110 - protein, 306 aa, synthetic construct
  REGION 1..306: note = Synthetic
  VARIANT 116: note = X is Ala, Arg, Asn, Cys, Glu, Gln, Gly, His, Ile, Leu, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRXWSAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 111 - protein, 306 aa, synthetic construct
  REGION 1..306: note = Synthetic
  VARIANT 117: note = X is Ala, Arg, Asn, Asp, Cys, Glu, Gln, Gly, His, Ile, Leu, Lys, Met, Phe, Pro, Ser, Thr, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDXSAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 112 - protein, 306 aa, synthetic construct
  REGION 1..306: note = Synthetic
  VARIANT 118: note = X is Ala, Arg, Asn, Asp, Cys, Glu, Gln, Gly, His, Ile, Leu, Lys, Met, Phe, Pro, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWXAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 113 - protein, 306 aa, synthetic construct
  REGION 1..306: note = Synthetic
  VARIANT 119: note = X is Arg, Asn, Asp, Cys, Glu, Gln, Gly, His, Ile, Leu, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSXETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 114 - protein, 306 aa, synthetic construct
  REGION 1..306: note = Synthetic
  VARIANT 120: note = X is Ala, Arg, Asn, Asp, Cys, Gln, Gly, His, Ile, Leu, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSAXTLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 115 - protein, 306 aa, synthetic construct
  REGION 1..306: note = Synthetic
  VARIANT 121: note = X is Ala, Arg, Asn, Asp, Cys, Gln, Gly, His, Ile, Leu, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSAEXLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 116 - protein, 306 aa, synthetic construct
  REGION 1..306: note = Synthetic
  VARIANT 122: note = X is Ala, Arg, Asn, Asp, Cys, Glu, Gln, Gly, His, Ile, Lys, Met, Phe, Pro, Ser, Thr, Trp, Tyr, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSAETXYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 117 - protein, 306 aa, synthetic construct
  REGION 1..306: note = Synthetic
  VARIANT 123: note = X is Ala, Arg, Asn, Asp, Cys, Glu, Gln, Gly, His, Ile, Leu, Lys, Met, Phe, Pro, Ser, Thr, Trp, or Val.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSSRDWSAETLXFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 118 - protein, 23 aa, Homo sapiens
MTEYKLVVVGAGGVGKSALTIQL

SEQ ID NO: 119 - protein, 23 aa, Homo sapiens
MTEYKLVVVGADGVGKSALTIQL

SEQ ID NO: 120 - protein, 23 aa, Homo sapiens
MTEYKLVVVGAVGVGKSALTIQL

SEQ ID NO: 121 - protein, 188 aa, Homo sapiens [sequence omitted]

SEQ ID NO: 122 - protein, 188 aa, Homo sapiens [sequence omitted]

SEQ ID NO: 123 - protein, 188 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 124 - protein, 9 aa, Homo sapiens
VVVGADGVG

SEQ ID NO: 125 - protein, 6 aa, Mus musculus
DPNSYY

SEQ ID NO: 126 - protein, 7 aa, Mus musculus
VFSSTEI

SEQ ID NO: 127 - protein, 16 aa, Mus musculus
CAVSGGTNSAGNKLTF

SEQ ID NO: 128 - protein, 5 aa, Mus musculus
LGHDT

SEQ ID NO: 129 - protein, 6 aa, Mus musculus
YNNKQL

SEQ ID NO: 130 - protein, 14 aa, Mus musculus
CASSRDWGPAEQFF

SEQ ID NO: 131 - protein, 137 aa, Mus musculus
MKTVTGPLFLCFWLQLNCVSRGEQVEQRPPHLSVREGDSAVITCTYTDPNSYYFFWYKQEPGASLQLLMKVFSSTEINEGQGFTVLLNKKDKRLSLNLTAAHPGDSAAYFCAVSGGTNSAGNKLTFGIGTRVLVRPD

SEQ ID NO: 132 - protein, 133 aa, Mus musculus
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSRDWGPAEQFFGPGTRLTVLE

SEQ ID NO: 133 - protein, 273 aa, Mus musculus
MKTVTGPLFLCFWLQLNCVSRGEQVEQRPPHLSVREGDSAVITCTYTDPNSYYFFWYKQEPGASLQLLMKVFSSTEINEGQGFTVLLNKKDKRLSLNLTAAHPGDSAAYFCAVSGGTNSAGNKLTFGIGTRVLVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 134 - protein, 305 aa, Mus musculus
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSRDWGPAEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 135 - protein, 136 aa, Mus musculus; identical to SEQ ID NO: 13

SEQ ID NO: 136 - protein, 172 aa, Mus musculus; identical to SEQ ID NO: 14

SEQ ID NO: 137 - protein, 6 aa, Mus musculus
NDMFDY

SEQ ID NO: 138 - protein, 7 aa, Mus musculus
VRSNVDK

SEQ ID NO: 139 - protein, 15 aa, Mus musculus
CAAGDSGGSNYKLTF

SEQ ID NO: 140 - protein, 5 aa, Mus musculus
NSHNY

SEQ ID NO: 141 - protein, 6 aa, Mus musculus
SYGAGN

SEQ ID NO: 142 - protein, 14 aa, Mus musculus
CASASWGGYAEQFF

SEQ ID NO: 143 - protein, 141 aa, Mus musculus
MTGFLKALLLVLCLRPEWIKSQQKTGGQQVKQSSPSLTVQEGGILILNCDYENDMFDYFAWYKKYPDNSPTLLISVRSNVDKREDGRFTVFLNKSGKHFSLHITASQPEDTAVYLCAAGDSGGSNYKLTFGKGTLLTVTPN

SEQ ID NO: 144 - protein, 131 aa, Mus musculus
MGSRLFLVLSLLCTKHMEAAVTQSPRNKVTVTGGNVTLSCRQTNSHNYMYWYRQDTGHGLRLIHYSYGAGNLQIGDVPDGYKATRTTQEDFFLLLELASPSQTSLYFCASASWGGYAEQFFGPGTRLTVLE

SEQ ID NO: 145 - protein, 277 aa, Mus musculus
MTGFLKALLLVLCLRPEWIKSQQKTGGQQVKQSSPSLTVQEGGILILNCDYENDMFDYFAWYKKYPDNSPTLLISVRSNVDKREDGRFTVFLNKSGKHFSLHITASQPEDTAVYLCAAGDSGGSNYKLTFGKGTLLTVTPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 146 - protein, 303 aa, Mus musculus
MGSRLFLVLSLLCTKHMEAAVTQSPRNKVTVTGGNVTLSCRQTNSHNYMYWYRQDTGHGLRLIHYSYGAGNLQIGDVPDGYKATRTTQEDFFLLLELASPSQTSLYFCASASWGGYAEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 147 - protein, 136 aa, Mus musculus; identical to SEQ ID NO: 13

SEQ ID NO: 148 - protein, 172 aa, Mus musculus; identical to SEQ ID NO: 14

SEQ ID NO: 149 - protein, 5 aa, Mus musculus
TTMRS

SEQ ID NO: 150 - protein, 5 aa, Mus musculus
LASGT

SEQ ID NO: 151 - protein, 15 aa, Mus musculus
CAADSSNTGYQNFYF

SEQ ID NO: 152 - protein, 5 aa, Mus musculus
SGHLS

SEQ ID NO: 153 - protein, 6 aa, Mus musculus
HYDKME

SEQ ID NO: 154 - protein, 15 aa, Mus musculus
CASSLTDPLDSDYTF

SEQ ID NO: 155 - protein, 133 aa, Mus musculus
MQRNLGAVLGILWVQICWVRGDQVEQSPSALSLHEGTDSALRCNFTTTMRSVQWFRQNSRGSLISLFYLASGTKENGRLKSAFDSKERRYSTLHIRDAQLEDSGTYFCAADSSNTGYQNFYFGKGTSLTVIPN

SEQ ID NO: 156 - protein, 143 aa, Mus musculus
MSNTAFPDPAWNTTLLSWVALFLLGTSSANSGVVQSPRYIIKGKGERSILKCIPISGHLSVAWYQQTQGQELKFFIQHYDKMERDKGNLPSRFSVQQFDDYHSEMNMSALELEDSAVYFCASSLTDPLDSDYTFGSGTRLLVI

SEQ ID NO: 157 - protein, 269 aa, Mus musculus
MQRNLGAVLGILWVQICWVRGDQVEQSPSALSLHEGTDSALRCNFTTTMRSVQWFRQNSRGSLISLFYLASGTKENGRLKSAFDSKERRYSTLHIRDAQLEDSGTYFCAADSSNTGYQNFYFGKGTSLTVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 158 - protein, 316 aa, Mus musculus
MSNTAFPDPAWNTTLLSWVALFLLGTSSANSGVVQSPRYIIKGKGERSILKCIPISGHLSVAWYQQTQGQELKFFIQHYDKMERDKGNLPSRFSVQQFDDYHSEMNMSALELEDSAVYFCASSLTDPLDSDYTFGSGTRLLVIEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 159 - protein, 136 aa, Mus musculus; identical to SEQ ID NO: 13

SEQ ID NO: 160 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 161 - protein, 13 aa, Mus musculus
CAALNTGYQNFYF

SEQ ID NO: 162 - protein, 612 aa, synthetic construct
  REGION 1..612: note = Synthetic
MQRNLGAVLGILWVQICWVRGDQVEQSPSALSLHEGTDSALRCNFTTTMRSVQWFRQNSRGSLISLFYLASGTKENGRLKSAFDSKERRYSTLHIRDAQLEDSGTYFCAADSSNTGYQNFYFGKGTSLTVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMSNTAFPDPAWNTTLLSWVALFLLGTSSANSGVVQSPRYIIKGKGERSILKCIPISGHLSVAWYQQTQGQELKFFIQHYDKMERDKGNLPSRFSVQQFDDYHSEMNMSALELEDSAVYFCASSLTDPLDSDYTFGSGTRLLVIEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 163 - DNA, 1839 bp, synthetic construct
  misc_feature 1..1839: note = Synthetic
  source 1..1839: mol_type = other DNA
atgcagaggaacctgggagctgtgctggggattctgtgggtgcagatttgctgggtgagaggggatcaggtggagcagagtccttcagccctgagcctccacgagggaaccgattctgctctgagatgcaattttacgaccaccatgaggagtgtgcagtggttccgacagaattccaggggcagcctcatcagtttgttctacttggcttcaggaacaaaggagaatgggaggctaaagtcagcatttgattctaaggagcggcgctacagcaccctgcacatcagggatgcccagctggaggactcaggcacttacttctgtgctgctgactcttcgaacacgggttaccagaacttctattttgggaaaggaacaagtttgactgtcattccaaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctgaactttcaaaacctgtcagttatgggactccgaatcctcctgctgaaagtagcgggatttaacctgctcatgacgctgaggctgtggtccagtcgggccaagcggtccggatccggagccaccaacttcagcctgctgaagcaggccggcgacgtggaggagaaccccggccccatgtctaacactgccttccctgaccccgcctggaacaccaccctgctatcttgggttgctctctttctcctgggaacaagttcagcaaattctggggttgtccagtctccaagatacataatcaaaggaaagggagaaaggtccattctaaaatgtattcccatctctggacatctctctgtggcctggtatcaacagactcaggggcaggaactaaagttcttcattcagcattatgataaaatggagagagataaaggaaacctgcccagcagattctcagtccaacagtttgatgactatcactctgagatgaacatgagtgccttggagctagaggactctgccgtgtacttctgtgccagctctctcacagatccgctagactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaattcatga

SEQ ID NO: 164 - DNA, 15 bp, synthetic construct
  misc_feature 1..15: note = Synthetic
  source 1..15: mol_type = other DNA
accaccatgaggagt

SEQ ID NO: 165 - DNA, 15 bp, synthetic construct
  misc_feature 1..15: note = Synthetic
  source 1..15: mol_type = other DNA
ttggcttcaggaaca

SEQ ID NO: 166 - DNA, 39 bp, synthetic construct
  misc_feature 1..39: note = Synthetic
  source 1..39: mol_type = other DNA
gctgctgactcttcgaacacgggttaccagaacttctat

SEQ ID NO: 167 - DNA, 15 bp, synthetic construct
  misc_feature 1..15: note = Synthetic
  source 1..15: mol_type = other DNA
tctggacatctctct

SEQ ID NO: 168 - DNA, 18 bp, synthetic construct
  misc_feature 1..18: note = Synthetic
  source 1..18: mol_type = other DNA
cattatgataaaatggag

SEQ ID NO: 169 - DNA, 39 bp, synthetic construct
  misc_feature 1..39: note = Synthetic
  source 1..39: mol_type = other DNA
gccagctctctcacagatccgctagactccgactacacc

SEQ ID NO: 170 - DNA, 399 bp, synthetic construct
  misc_feature 1..399: note = Synthetic
  source 1..399: mol_type = other DNA
atgcagaggaacctgggagctgtgctggggattctgtgggtgcagatttgctgggtgagaggggatcaggtggagcagagtccttcagccctgagcctccacgagggaaccgattctgctctgagatgcaattttacgaccaccatgaggagtgtgcagtggttccgacagaattccaggggcagcctcatcagtttgttctacttggcttcaggaacaaaggagaatgggaggctaaagtcagcatttgattctaaggagcggcgctacagcaccctgcacatcagggatgcccagctggaggactcaggcacttacttctgtgctgctgactcttcgaacacgggttaccagaacttctattttgggaaaggaacaagtttgactgtcattccaaac

SEQ ID NO: 171 - DNA, 429 bp, synthetic construct
  misc_feature 1..429: note = Synthetic
  source 1..429: mol_type = other DNA
atgtctaacactgccttccctgaccccgcctggaacaccaccctgctatcttgggttgctctctttctcctgggaacaagttcagcaaattctggggttgtccagtctccaagatacataatcaaaggaaagggagaaaggtccattctaaaatgtattcccatctctggacatctctctgtggcctggtatcaacagactcaggggcaggaactaaagttcttcattcagcattatgataaaatggagagagataaaggaaacctgcccagcagattctcagtccaacagtttgatgactatcactctgagatgaacatgagtgccttggagctagaggactctgccgtgtacttctgtgccagctctctcacagatccgctagactccgactacaccttcggctcagggaccaggcttttggtaata

SEQ ID NO: 172 - DNA, 807 bp, synthetic construct
  misc_feature 1..807: note = Synthetic
  source 1..807: mol_type = other DNA
atgcagaggaacctgggagctgtgctggggattctgtgggtgcagatttgctgggtgagaggggatcaggtggagcagagtccttcagccctgagcctccacgagggaaccgattctgctctgagatgcaattttacgaccaccatgaggagtgtgcagtggttccgacagaattccaggggcagcctcatcagtttgttctacttggcttcaggaacaaaggagaatgggaggctaaagtcagcatttgattctaaggagcggcgctacagcaccctgcacatcagggatgcccagctggaggactcaggcacttacttctgtgctgctgactcttcgaacacgggttaccagaacttctattttgggaaaggaacaagtttgactgtcattccaaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctgaactttcaaaacctgtcagttatgggactccgaatcctcctgctgaaagtagcgggatttaacctgctcatgacgctgaggctgtggtccagt

SEQ ID NO: 173 - DNA, 951 bp, synthetic construct
  misc_feature 1..951: note = Synthetic
  source 1..951: mol_type = other DNA
atgtctaacactgccttccctgaccccgcctggaacaccaccctgctatcttgggttgctctctttctcctgggaacaagttcagcaaattctggggttgtccagtctccaagatacataatcaaaggaaagggagaaaggtccattctaaaatgtattcccatctctggacatctctctgtggcctggtatcaacagactcaggggcaggaactaaagttcttcattcagcattatgataaaatggagagagataaaggaaacctgcccagcagattctcagtccaacagtttgatgactatcactctgagatgaacatgagtgccttggagctagaggactctgccgtgtacttctgtgccagctctctcacagatccgctagactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaattcatga

SEQ ID NO: 174 - DNA, 408 bp, synthetic construct
  misc_feature 1..408: note = Synthetic
  source 1..408: mol_type = other DNA
atccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagt

SEQ ID NO: 175 - DNA, 522 bp, synthetic construct
  misc_feature 1..522: note = Synthetic
  source 1..522: mol_type = other DNA
gaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaattcatga

SEQ ID NO: 176 - DNA, 1818 bp, synthetic construct
  misc_feature 1..1818: note = Synthetic
  source 1..1818: mol_type = other DNA
atgaagacggtgactggacctttgttcctgtgcttctggctgcagctgaactgtgtgagcagaggcgagcaggtggagcagcgccctcctcacctgagtgtccgggagggagacagtgccgttatcacctgcacctacacagaccctaacagttattacttcttctggtacaagcaagagccgggggcaagtcttcagttgcttatgaaggttttctcaagtacggaaataaacgaaggacaaggattcactgtcctactgaacaagaaagacaaacgactctctctgaacctcacagctgcccatcctggggactcagccgcgtacttctgcgcagtcagtggagggactaacagtgcagggaacaagctaacttttggaattggaaccagggtgctggtcaggccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagtcgggccaagcggtccggatccggagccaccaacttcagcctgctgaagcaggccggcgacgtggaggagaaccccggccccatgggctgtaggctcctaagctgtgtggccttctgcctcttgggaataggccctttggagacggctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagtcgggactgggggcctgctgagcagttcttcggaccagggacacgactcaccgtcctagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctga

SEQ ID NO: 177 - DNA, 18 bp, synthetic construct
  misc_feature 1..18: note = Synthetic
  source 1..18: mol_type = other DNA
gaccctaacagttattac

SEQ ID NO: 178 - DNA, 21 bp, synthetic construct
  misc_feature 1..21: note = Synthetic
  source 1..21: mol_type = other DNA
gttttctcaagtacggaaata

SEQ ID NO: 179 - DNA, 48 bp, synthetic construct
  misc_feature 1..48: note = Synthetic
  source 1..48: mol_type = other DNA
tgcgcagtcagtggagggactaacagtgcagggaacaagctaactttt

SEQ ID NO: 180 - DNA, 15 bp, synthetic construct
  misc_feature 1..15: note = Synthetic
  source 1..15: mol_type = other DNA
ctgggccacgatact

SEQ ID NO: 181 - DNA, 18 bp, synthetic construct
  misc_feature 1..18: note = Synthetic
  source 1..18: mol_type = other DNA
tacaataataagcaactc

SEQ ID NO: 182 - DNA, 42 bp, synthetic construct
  misc_feature 1..42: note = Synthetic
  source 1..42: mol_type = other DNA
tgtgccagcagtcgggactgggggcctgctgagcagttcttc

SEQ ID NO: 183 - DNA, 411 bp, synthetic construct
  misc_feature 1..411: note = Synthetic
  source 1..411: mol_type = other DNA
atgaagacggtgactggacctttgttcctgtgcttctggctgcagctgaactgtgtgagcagaggcgagcaggtggagcagcgccctcctcacctgagtgtccgggagggagacagtgccgttatcacctgcacctacacagaccctaacagttattacttcttctggtacaagcaagagccgggggcaagtcttcagttgcttatgaaggttttctcaagtacggaaataaacgaaggacaaggattcactgtcctactgaacaagaaagacaaacgactctctctgaacctcacagctgcccatcctggggactcagccgcgtacttctgcgcagtcagtggagggactaacagtgcagggaacaagctaacttttggaattggaaccagggtgctggtcaggccagac

SEQ ID NO: 184 - DNA, 399 bp, synthetic construct
  misc_feature 1..399: note = Synthetic
  source 1..399: mol_type = other DNA
atgggctgtaggctcctaagctgtgtggccttctgcctcttgggaataggccctttggagacggctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagtcgggactgggggcctgctgagcagttcttcggaccagggacacgactcaccgtcctagag

SEQ ID NO: 185 - DNA, 819 bp, synthetic construct
  misc_feature 1..819: note = Synthetic
  source 1..819: mol_type = other DNA
atgaagacggtgactggacctttgttcctgtgcttctggctgcagctgaactgtgtgagcagaggcgagcaggtggagcagcgccctcctcacctgagtgtccgggagggagacagtgccgttatcacctgcacctacacagaccctaacagttattacttcttctggtacaagcaagagccgggggcaagtcttcagttgcttatgaaggttttctcaagtacggaaataaacgaaggacaaggattcactgtcctactgaacaagaaagacaaacgactctctctgaacctcacagctgcccatcctggggactcagccgcgtacttctgcgcagtcagtggagggactaacagtgcagggaacaagctaacttttggaattggaaccagggtgctggtcaggccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagt

SEQ ID NO: 186 - DNA, 918 bp, synthetic construct
  misc_feature 1..918: note = Synthetic
  source 1..918: mol_type = other DNA
atgggctgtaggctcctaagctgtgtggccttctgcctcttgggaataggccctttggagacggctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagtcgggactgggggcctgctgagcagttcttcggaccagggacacgactcaccgtcctagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctga

SEQ ID NO: 187 - DNA, 408 bp, synthetic construct; identical to SEQ ID NO: 174
  misc_feature 1..408: note = Synthetic
  source 1..408: mol_type = other DNA

SEQ ID NO: 188 - DNA, 519 bp, synthetic construct
  misc_feature 1..519: note = Synthetic
  source 1..519: mol_type = other DNA
gatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctga

SEQ ID NO: 189 - DNA, 18 bp, synthetic construct
  misc_feature 1..18: note = Synthetic
  source 1..18: mol_type = other DNA
aatgatatgtttgactat

SEQ ID NO: 190 - DNA, 21 bp, synthetic construct
  misc_feature 1..21: note = Synthetic
  source 1..21: mol_type = other DNA
gtacgctcaaatgtggataag

SEQ ID NO: 191 - DNA, 45 bp, synthetic construct
  misc_feature 1..45: note = Synthetic
  source 1..45: mol_type = other DNA
tgcgcagcaggtgacagtggaggcagcaattacaaactgacattt

SEQ ID NO: 192 - DNA, 15 bp, synthetic construct
  misc_feature 1..15: note = Synthetic
  source 1..15: mol_type = other DNA
aatagccacaactac

SEQ ID NO: 193 - DNA, 18 bp, synthetic construct
  misc_feature 1..18: note = Synthetic
  source 1..18: mol_type = other DNA
tcatatggtgctggcaac

SEQ ID NO: 194 - DNA, 42 bp, synthetic construct
  misc_feature 1..42: note = Synthetic
  source 1..42: mol_type = other DNA
tgtgccagcgcgagctgggggggctatgctgagcagttcttc

SEQ ID NO: 195 - DNA, 423 bp, synthetic construct
  misc_feature 1..423: note = Synthetic
  source 1..423: mol_type = other DNA
atgactggcttcctgaaggccttgctgttggttctgtgcctgcggccagaatggataaagagtcaacagaagactggtggccagcaagttaaacaaagctctccatcgctgactgttcaagagggagggatattgatcctgaattgtgattacgagaatgatatgtttgactattttgcctggtacaaaaaataccctgacaacagccccacactcctgatatccgtacgctcaaatgtggataagagggaagacggaagattcacagttttcttgaacaaaagcggcaaacacttctcactgcacatcacagcctcccagcctgaagacacagcagtgtacctctgcgcagcaggtgacagtggaggcagcaattacaaactgacatttgggaaaggaactctcttaactgtgactccaaac

SEQ ID NO: 196 - DNA, 393 bp, synthetic construct
  misc_feature 1..393: note = Synthetic
  source 1..393: mol_type = other DNA
atgggctccaggctctttctggtcttgagcctcctgtgtacaaaacacatggaggctgcagtcacccaaagccctagaaacaaggtgacagtaacaggaggaaacgtgacattgagctgtcgccagactaatagccacaactacatgtactggtatcggcaggacactgggcatgggctgaggctgatccattactcatatggtgctggcaaccttcaaataggagatgtccctgatgggtacaaggccaccagaacaacgcaagaagacttcttcctcctgctggaattggcttctccctctcagacatctttgtacttctgtgccagcgcgagctgggggggctatgctgagcagttcttcggaccagggacacgactcaccgtcctagag

SEQ ID NO: 197 - DNA, 831 bp, synthetic construct
  misc_feature 1..831: note = Synthetic
  source 1..831: mol_type = other DNA
atgactggcttcctgaaggccttgctgttggttctgtgcctgcggccagaatggataaagagtcaacagaagactggtggccagcaagttaaacaaagctctccatcgctgactgttcaagagggagggatattgatcctgaattgtgattacgagaatgatatgtttgactattttgcctggtacaaaaaataccctgacaacagccccacactcctgatatccgtacgctcaaatgtggataagagggaagacggaagattcacagttttcttgaacaaaagcggcaaacacttctcactgcacatcacagcctcccagcctgaagacacagcagtgtacctctgcgcagcaggtgacagtggaggcagcaattacaaactgacatttgggaaaggaactctcttaactgtgactccaaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagt

SEQ ID NO: 198 - DNA, 912 bp, synthetic construct
  misc_feature 1..912: note = Synthetic
  source 1..912: mol_type = other DNA
atgggctccaggctctttctggtcttgagcctcctgtgtacaaaacacatggaggctgcagtcacccaaagccctagaaacaaggtgacagtaacaggaggaaacgtgacattgagctgtcgccagactaatagccacaactacatgtactggtatcggcaggacactgggcatgggctgaggctgatccattactcatatggtgctggcaaccttcaaataggagatgtccctgatgggtacaaggccaccagaacaacgcaagaagacttcttcctcctgctggaattggcttctccctctcagacatctttgtacttctgtgccagcgcgagctgggggggctatgctgagcagttcttcggaccagggacacgactcaccgtcctagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctga

SEQ ID NO: 199 - DNA, 408 bp, synthetic construct; identical to SEQ ID NO: 174
  misc_feature 1..408: note = Synthetic
  source 1..408: mol_type = other DNA

SEQ ID NO: 200 - DNA, 519 bp, synthetic construct; identical to SEQ ID NO: 188
  misc_feature 1..519: note = Synthetic
  source 1..519: mol_type = other DNA

SEQ ID NO: 201 - protein, 605 aa, synthetic construct
  REGION 1..605: note = Synthetic
MKTVTGPLFLCFWLQLNCVSRGEQVEQRPPHLSVREGDSAVITCTYTDPNSYYFFWYKQEPGASLQLLMKVFSSTEINEGQGFTVLLNKKDKRLSLNLTAAHPGDSAAYFCAVSGGTNSAGNKLTFGIGTRVLVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSRDWGPAEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 202 - DNA, 1824 bp, synthetic construct
  misc_feature 1..1824: note = Synthetic
  source 1..1824: mol_type = other DNA
atgactggcttcctgaaggccttgctgttggttctgtgcctgcggccagaatggataaagagtcaacagaagactggtggccagcaagttaaacaaagctctccatcgctgactgttcaagagggagggatattgatcctgaattgtgattacgagaatgatatgtttgactattttgcctggtacaaaaaataccctgacaacagccccacactcctgatatccgtacgctcaaatgtggataagagggaagacggaagattcacagttttcttgaacaaaagcggcaaacacttctcactgcacatcacagcctcccagcctgaagacacagcagtgtacctctgcgcagcaggtgacagtggaggcagcaattacaaactgacatttgggaaaggaactctcttaactgtgactccaaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagtcgggccaagcggtccggatccggagccaccaacttcagcctgctgaagcaggccggcgacgtggaggagaaccccggccccatgggctccaggctctttctggtcttgagcctcctgtgtacaaaacacatggaggctgcagtcacccaaagccctagaaacaaggtgacagtaacaggaggaaacgtgacattgagctgtcgccagactaatagccacaactacatgtactggtatcggcaggacactgggcatgggctgaggctgatccattactcatatggtgctggcaaccttcaaataggagatgtccctgatgggtacaaggccaccagaacaacgcaagaagacttcttcctcctgctggaattggcttctccctctcagacatctttgtacttctgtgccagcgcgagctgggggggctatgctgagcagttcttcggaccagggacacgactcaccgtcctagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctga

SEQ ID NO: 203 - protein, 607 aa, synthetic construct
  REGION 1..607: note = Synthetic
MTGFLKALLLVLCLRPEWIKSQQKTGGQQVKQSSPSLTVQEGGILILNCDYENDMFDYFAWYKKYPDNSPTLLISVRSNVDKREDGRFTVFLNKSGKHFSLHITASQPEDTAVYLCAAGDSGGSNYKLTFGKGTLLTVTPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMGSRLFLVLSLLCTKHMEAAVTQSPRNKVTVTGGNVTLSCRQTNSHNYMYWYRQDTGHGLRLIHYSYGAGNLQIGDVPDGYKATRTTQEDFFLLLELASPSQTSLYFCASASWGGYAEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 204 - protein, 16 aa, Mus musculus
CAVSGGTNSAGNKLTF

SEQ ID NO: 205 - protein, 14 aa, Mus musculus
CASASWGGYAEQFF

SEQ ID NO: 206 - protein, 14 aa, Mus musculus
CTCSADRGAETLYF

SEQ ID NO: 207 - protein, 15 aa, Mus musculus
  VARIANT 9: note = Wherein X is alanine, arginine, asparagine, asparatic acid, cysteine, glutamic acid, glutamine, histidine, isoleucine, leucine, lysine, methionine, phenylalanine, proline, serine, threonine, tryptophan, tyrosine, or valine
CAADSSNTXYQNFYF

SEQ ID NO: 208 - protein, 133 aa, Mus musculus
  VARIANT 116: note = Wherein X is alanine, arginine, asparagine, asparatic acid, cysteine, glutamic acid, glutamine, histidine, isoleucine, leucine, lysine, methionine, phenylalanine, proline, serine, threonine, tryptophan, tyrosine, or valine
MQRNLGAVLGILWVQICWVRGDQVEQSPSALSLHEGTDSALRCNFTTTMRSVQWFRQNSRGSLISLFYLASGTKENGRLKSAFDSKERRYSTLHIRDAQLEDSGTYFCAADSSNTXYQNFYFGKGTSLTVIPN

SEQ ID NO: 209 - protein, 269 aa, Mus musculus
  VARIANT 116: note = Wherein X is alanine, arginine, asparagine, asparatic acid, cysteine, glutamic acid, glutamine, histidine, isoleucine, leucine, lysine, methionine, phenylalanine, proline, serine, threonine, tryptophan, tyrosine, or valine
MQRNLGAVLGILWVQICWVRGDQVEQSPSALSLHEGTDSALRCNFTTTMRSVQWFRQNSRGSLISLFYLASGTKENGRLKSAFDSKERRYSTLHIRDAQLEDSGTYFCAADSSNTXYQNFYFGKGTSLTVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

(19) United States
(12) Patent Application Publication
(10) Pub. No.: US 2025/0263462 A1
(43) Pub. Date: Aug. 21, 2025

(54) ANTI-MUTATED KRAS T CELL RECEPTORS

(71) Applicant: The United States of America, as represented by the Secretary, Department of Health and Human Servic, Bethesda, MD (US)

(72) Inventors: Qiong J. Wang, Highland Park, NJ (US); Zhiya Yu, Potomac, MD (US); James C. Yang, Bethesda, MD (US)

(73) Assignee: The United States of America, as represented by the Secretary, Department of Health and Human servic, Bethesda, MD (US)

(21) Appl. No.: 19/185,602

(22) Filed: Apr. 22, 2025

Related U.S. Application Data
(63) Continuation of application No. 17/535,318, filed on Nov. 24, 2021, now Pat. No. 12,312,391.
Application No. 17/535,318 is a continuation of application No. 15/528,813, filed on May 23, 2017, now Pat. No. 11,207,394.
(60) Provisional application No. 62/171,321, filed on Jun. 5, 2015.
(60) Provisional application No. 62/084,654, filed on Nov. 26, 2014.

(51) Int. Cl. C07K 14/725; A61K 38/00; A61K 39/00; A61K 40/10; A61K 40/32; A61K 40/42; C07K 14/82; G01N 33/574

(52) U.S. Cl. CPC C07K 14/7051; A61K 39/001164; A61K 40/10; A61K 40/32; A61K 40/4253; C07K 14/82; C12Y 306/00; G01N 33/5748; A61K 38/00; A61K 2239/38; A61K 2239/54; G01N 2333/914

(57) ABSTRACT

Disclosed is an isolated or purified T cell receptor (TCR) having antigenic specificity for an HLA-A11-restricted epitope of mutated Kirsten rat sarcoma viral oncogene homolog (KRAS) (KRAS₇₋₁₆), Neuroblastoma RAS Viral (V-Ras) Oncogene Homolog (NRAS), or Harvey Rat Sarcoma Viral Oncogene Homolog (HRAS). Related polypeptides and proteins, as well as related nucleic acids, recombinant expression vectors, host cells, populations of cells, and pharmaceutical compositions are also provided. Also disclosed are methods of detecting the presence of cancer in a mammal and methods of treating or preventing cancer in a mammal.

### CROSS-REFERENCE TO RELATED APPLICATIONS

This patent application is a continuation of co-pending U.S. patent application Ser. No. 17/535,318, filed Nov. 24, 2021, which is a continuation of U.S. patent application Ser. No. 15/528,813, filed May 23, 2017, now U.S. Pat. No. 11,207,394, which is the U.S. national stage of PCT/US2015/062269, filed Nov. 24, 2015, which claims the benefit of U.S. Provisional Patent Application No. 62/084,654, filed Nov. 26, 2014 and U.S. Provisional Patent Application No. 62/171,321, filed Jun. 5, 2015, each of which is incorporated by reference in its entirety herein.

### STATEMENT REGARDING FEDERALLY SPONSORED RESEARCH OR DEVELOPMENT

This invention was made with Government support under project number Z01BC011337-04 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 304,688 Byte Extensible Markup Language (XML) file named “772939_ST26.xml,” dated Apr. 17, 2025.

### BACKGROUND OF THE INVENTION

Some cancers may have very limited treatment options, particularly when the cancer becomes metastatic and unresectable. Despite advances in treatments such as, for example, surgery, chemotherapy, and radiation therapy, the prognosis for many cancers, such as, for example, pancreatic, colorectal, lung, endometrial, ovarian, and prostate cancers, may be poor. Accordingly, there exists an unmet need for additional treatments for cancer.

### BRIEF SUMMARY OF THE INVENTION

An embodiment of the invention provides an isolated or purified T cell receptor (TCR) having antigenic specificity for a mutated epitope, the mutated epitope (a) comprising VVVGADGVGK (SEQ ID NO: 2) or (b) consisting of VVVGAVGVGK (SEQ ID NO: 33) or VVGAVGVGK (SEQ ID NO: 35).

The invention further provides related polypeptides and proteins, as well as related nucleic acids, recombinant expression vectors, host cells, populations of cells, and pharmaceutical compositions relating to the TCRs of the invention.

Methods of detecting the presence of cancer in a mammal and methods of treating or preventing cancer in a mammal are further provided by the invention.

### DETAILED DESCRIPTION OF THE INVENTION

Kirsten rat sarcoma viral oncogene homolog (KRAS), also referred to as GTPase KRas, V-Ki-Ras2 Kirsten rat sarcoma viral oncogene, or KRAS2, is a member of the small GTPase superfamily. There are two transcript variants of KRAS: KRAS variant A and KRAS variant B. Hereinafter, references to “KRAS” (mutated or unmutated) refer to both variant A and variant B, unless specified otherwise. Without being bound to a particular theory or mechanism, it is believed that, when mutated, KRAS may be involved in signal transduction early in the oncogenesis of many human cancers. A single amino acid substitution may activate the mutation. When activated, mutated KRAS binds to guanosine-5′-triphosphate (GTP) and converts GTP to guanosine 5′-diphosphate (GDP). The mutated KRAS protein product may be constitutively activated. Mutated KRAS protein may be expressed in any of a variety of human cancers such as, for example, pancreatic (e.g., pancreatic carcinoma), colorectal, lung (e.g., lung adenocarcinoma), endometrial, ovarian (e.g., epithelial ovarian cancer), and prostate cancers.

An embodiment of the invention provides an isolated or purified TCR having antigenic specificity for mutated human KRAS (hereinafter, “mutated KRAS”). Hereinafter, references to a “TCR” also refer to functional portions and functional variants of the TCR, unless specified otherwise. The inventive TCR may have antigenic specificity for any mutated KRAS protein, polypeptide or peptide. In an embodiment of the invention, the TCR has antigenic specificity for a mutated KRAS protein comprising or consisting of the amino acid sequence of SEQ ID NO: 1, 32, 122, or 123. The mutated KRAS variant A protein amino acid sequences of each of SEQ ID NOs: 1 and 32 generally corresponds to positions 1-189 of the unmutated, wild-type (WT) KRAS protein variant A amino acid sequence of SEQ ID NO: 29 with the exception that in SEQ ID NOs: 1 and 32, the glycine at position 12 is substituted with aspartic acid or valine, respectively. The mutated KRAS variant B protein amino acid sequences of each of SEQ ID NOs: 122 and 123 generally corresponds to positions 1-188 of the unmutated. WT KRAS protein variant B amino acid sequence of SEQ ID NO: 121 with the exception that in SEQ ID NOs: 122 and 123, the glycine at position 12 is substituted with aspartic acid or valine, respectively. In a preferred embodiment of the invention, the TCR has antigenic specificity for a mutated KRAS7-16 peptide comprising or consisting of the amino acid sequence of VVVGADGVGK (SEQ ID NO: 2), or VVVGAVGVGK (SEQ ID NO: 33). The mutated KRAS peptide amino acid sequences of SEQ ID NOs: 2 and 33 generally correspond to positions 1-10 of the unmutated. WT KRAS7-16 peptide amino acid sequence of SEQ ID NO: 30 with the exception that in SEQ ID NOs: 2 and 33, the glycine at position 6 is substituted with aspartic acid or valine, respectively. In an embodiment of the invention, the TCR has antigenic specificity for a mutated KRAS8-16 peptide comprising or consisting of the amino acid sequence of VVGADGVGK (SEQ ID NO: 34) or VVGAVGVGK (SEQ ID NO: 35). The mutated KRAS peptide amino acid sequences of SEQ ID NOs: 34 and 35 generally correspond to positions 1-9 of the unmutated. WT KRAS8-16 peptide amino acid sequence of SEQ ID NO: 31 with the exception that in SEQ ID NOs: 34 and 35, the glycine at position 5 is substituted with aspartic acid or valine, respectively. In a preferred embodiment, the TCR has antigenic specificity for a mutated KRAS epitope, the mutated KRAS epitope (a) comprising VVVGADGVGK (SEQ ID NO: 2) or (b) consisting of VVVGAVGVGK (SEQ ID NO: 33) or VVGAVGVGK (SEQ ID NO: 35). In an especially preferred embodiment, the TCR has antigenic specificity for a mutated KRAS epitope comprising VVVGADGVGK (SEQ ID NO: 2). In another preferred embodiment, the TCR has antigenic specificity for a mutated KRAS epitope consisting of VVVGAVGVGK (SEQ ID NO: 33) or VVGAVGVGK (SEQ ID NO: 35). The mutated KRAS amino acid sequences VVVGAVGVGK (SEQ ID NO: 33) and VVGAVGVGK (SEQ ID NO: 35) are also referred to herein as “KRAS G12V.” The mutated KRAS amino acid sequences VVVGADGVGK (SEQ ID NO: 2) and VVGADGVGK (SEQ ID NO: 34) are also referred to herein as “KRAS G12D.”

The mutated KRAS epitope amino acid sequences described herein are also found in two other mutated oncogenes in human cancer. Neuroblastoma RAS Viral (V-Ras) Oncogene Homolog (NRAS) and Harvey Rat Sarcoma Viral Oncogene Homolog (HRAS). The amino acid sequences of mutated human NRAS and mutated human HRAS contain the mutated human KRAS epitope sequences described herein. Accordingly, in an embodiment of the invention, the inventive TCRs also have antigenic specificity for mutated human NRAS and HRAS. Mutated human KRAS, mutated human NRAS, and mutated human HRAS are collectively referred to herein as “mutated target(s).”

In an embodiment of the invention, the inventive TCRs are able to recognize mutated target, e.g., mutated KRAS, in a major histocompatibility complex (MHC) class I-dependent manner. “MHC class I-dependent manner.” as used herein, means that the TCR elicits an immune response upon binding to mutated target, e.g., mutated KRAS, within the context of an MHC class I molecule. The MHC class I molecule can be any MHC class I molecule known in the art, e.g., HLA-A molecules. In a preferred embodiment of the invention, the TCR has antigenic specificity for the mutated epitope, presented in the context of an HLA-A11 molecule.

The TCRs of the invention provide many advantages, including when expressed by cells used for adoptive cell transfer. Mutated KRAS, mutated NRAS, and mutated HRAS are expressed by cancer cells and are not expressed by normal, noncancerous cells. Without being bound to a particular theory or mechanism, it is believed that the inventive TCRs advantageously target the destruction of cancer cells while minimizing or eliminating the destruction of normal, non-cancerous cells, thereby reducing, for example, by minimizing or eliminating, toxicity. Moreover, the inventive TCRs may, advantageously, successfully treat or prevent one or more of mutated KRAS-positive cancers, mutated NRAS-positive cancers, and mutated HRAS-positive cancers that do not respond to other types of treatment such as, for example, chemotherapy, surgery, or radiation. Additionally, the inventive TCRs may provide highly avid recognition of one or more of mutated KRAS, mutated NRAS, and mutated HRAS, which may provide the ability to recognize unmanipulated tumor cells (e.g., tumor cells that have not been treated with interferon (IFN)-γ, transfected with a vector encoding one or both of mutated KRAS and HLA-A11, pulsed with the mutated KRAS7-16 or KRAS8-16 peptide, or a combination thereof).

The phrase “antigenic specificity.” as used herein, means that the TCR can specifically bind to and immunologically recognize mutated target, e.g., mutated KRAS, with high avidity. For example, a TCR may be considered to have “antigenic specificity” for mutated target if T cells expressing the TCR secrete at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more. 10,000 pg/mL or more, 20,000 pg/mL or more, or a range defined by any two of the foregoing values) of IFN-γ upon co-culture with (a) antigen-negative HLA-A11⁺ target cells pulsed with a low concentration of mutated target peptide (e.g., about 0.05 ng/ml to about 5 ng/ml. 0.05 ng/ml, 0.1 ng/ml, 0.5 ng/mL, 1 ng/mL, 5 ng/ml, or a range defined by any two of the foregoing values) or (b) antigen-negative HLA-A11⁺ target cells into which a nucleotide sequence encoding the mutated target has been introduced such that the target cell expresses the mutated target. Cells expressing the inventive TCRs may also secrete IFN-γ upon co-culture with antigen-negative HLA-A11⁺ target cells pulsed with higher concentrations of mutated target peptide.

Alternatively or additionally, a TCR may be considered to have “antigenic specificity” for a mutated target if T cells expressing the TCR secrete at least twice as much IFN-γ upon co-culture with (a) antigen-negative HLA-A11⁺ target cells pulsed with a low concentration of mutated target peptide or (b) antigen-negative HLA-A11⁺ target cells into which a nucleotide sequence encoding the mutated target has been introduced such that the target cell expresses the mutated target as compared to the amount of IFN-γ expressed by a negative control. The negative control may be, for example, (i) T cells expressing the TCR, co-cultured with (a) antigen-negative HLA-A11⁺ target cells pulsed with the same concentration of an irrelevant peptide (e.g., some other peptide with a different sequence from the mutated target peptide) or (b) antigen-negative HLA-A11⁺ target cells into which a nucleotide sequence encoding an irrelevant peptide has been introduced such that the target cell expresses the irrelevant peptide, or (ii) untransduced T cells (e.g., derived from PBMC, which do not express the TCR) co-cultured with (a) antigen-negative HLA-A11⁺ target cells pulsed with the same concentration of mutated target peptide or (b) antigen-negative HLA-A11′ target cells into which a nucleotide sequence encoding the mutated target has been introduced such that the target cell expresses the mutated target. IFN-γ secretion may be measured by methods known in the art such as, for example, enzyme-linked immunosorbent assay (ELISA).

Alternatively or additionally, a TCR may be considered to have “antigenic specificity” for a mutated target if at least twice as many of the numbers of T cells expressing the TCR secrete IFN-γ upon co-culture with (a) antigen-negative HLA-A11⁺ target cells pulsed with a low concentration of mutated target peptide or (b) antigen-negative HLA-A11⁺ target cells into which a nucleotide sequence encoding the mutated target has been introduced such that the target cell expresses the mutated target as compared to the numbers of negative control T cells that secrete IFN-γ. The concentration of peptide and the negative control may be as described herein with respect to other aspects of the invention. The numbers of cells secreting IFN-γ may be measured by methods known in the art such as, for example, ELISPOT.

The invention provides a TCR comprising two polypeptides (i.e., polypeptide chains), such as an alpha (α) chain of a TCR, a beta (β) chain of a TCR, a gamma (γ) chain of a TCR, a delta (δ) chain of a TCR, or a combination thereof. The polypeptides of the inventive TCR can comprise any amino acid sequence, provided that the TCR has antigenic specificity for the mutated target, e.g., mutated KRAS.

In an embodiment of the invention, the TCR comprises two polypeptide chains, each of which comprises a variable region comprising a complementarity determining region (CDR) 1, a CDR2, and a CDR3 of a TCR. In an embodiment of the invention, the TCR comprises: (a) a first polypeptide chain comprising a CDR1 comprising the amino acid sequence of SEQ ID NO: 3 (CDR1 of a chain of anti-KRAS G12D TCR), a CDR2 comprising the amino acid sequence of SEQ ID NO: 4 (CDR2 of α chain of anti-KRAS G12D TCR), and a CDR3 comprising the amino acid sequence of SEQ ID NO: 5 (CDR3 of a chain of anti-KRAS G12D TCR), and a second polypeptide chain comprising a CDR1 comprising the amino acid sequence of SEQ ID NO: 6 (CDR1 of β chain of anti-KRAS G12D TCR), a CDR2 comprising the amino acid sequence of SEQ ID NO: 7 (CDR2 of β chain of anti-KRAS G12D TCR), and a CDR3 comprising the amino acid sequence of SEQ ID NO: 8 (CDR3 of β chain of anti-KRAS G12D TCR); (b) a first polypeptide chain comprising an anti-KRAS G12V TCR α chain CDR1 comprising the amino acid sequence of SEQ ID NO: 125, an anti-KRAS G12V TCR α chain CDR2 comprising the amino acid sequence of SEQ ID NO: 126, an anti-KRAS G12V TCR α chain CDR3 comprising the amino acid sequence of SEQ ID NO: 127, and a second polypeptide chain comprising an anti-KRAS G12V TCR β chain CDR1 comprising the amino acid sequence of SEQ ID NO: 128, an anti-KRAS G12V TCR β chain CDR2 comprising the amino acid sequence of SEQ ID NO: 129, and an anti-KRAS G12V TCR β chain CDR3 comprising the amino acid sequence of SEQ ID NO: 130; (c) a first polypeptide chain comprising an anti-KRAS G12V TCR α chain CDR1 comprising the amino acid sequence of SEQ ID NO: 137, an anti-KRAS G12V TCR α chain CDR2 comprising the amino acid sequence of SEQ ID NO: 138, an anti-KRAS G12V TCR α chain CDR3 comprising the amino acid sequence of SEQ ID NO: 139, and a second polypeptide chain comprising an anti-KRAS G12V TCR β chain CDR1 comprising the amino acid sequence of SEQ ID NO: 140, an anti-KRAS G12V TCR β chain CDR2 comprising the amino acid sequence of SEQ ID NO: 141, and an anti-KRAS G12V TCR β chain CDR3 comprising the amino acid sequence of SEQ ID NO: 142; or (d) first polypeptide chain comprising an anti-KRAS G12D TCR α chain CDR1 comprising the amino acid sequence of SEQ ID NO: 149, an anti-KRAS G12D TCR α chain CDR2 comprising the amino acid sequence of SEQ ID NO: 150, an anti-KRAS G12D TCR α chain CDR3 comprising the amino acid sequence of SEQ ID NO: 151, and a second polypeptide chain comprising an anti-KRAS G12D TCR β chain CDR1 comprising the amino acid sequence of SEQ ID NO: 152, an anti-KRAS G12D TCR β chain CDR2 comprising the amino acid sequence of SEQ ID NO: 153, and an anti-KRAS G12D TCR β chain CDR3 comprising the amino acid sequence of SEQ ID NO: 154. In this regard, the inventive TCR can comprise any one or more of the amino acid sequences selected from the group consisting of SEQ ID NOs: 3-8:125-130; 137-142; and 149-154. Preferably, the TCR comprises the amino acid sequences of SEQ ID NOs: 3-5: SEQ ID NOs: 6-8: SEQ ID NOS: 125-127: SEQ ID NOs: 128-130; SEQ ID NOs: 137-139; SEQ ID NOs: 140-142: SEQ ID NOs: 149-151; or SEQ ID NOs: 152-154. In an especially preferred embodiment, the TCR comprises the amino acid sequences of (a) all of SEQ ID NOs: 3-8; (b) all of SEQ ID NOS: 125-130; (c) all of SEQ ID NOs: 137-142; (d) all of SEQ ID NOs: 149-154.

In an embodiment of the invention, the TCR comprises an amino acid sequence of a variable region of a TCR comprising the CDRs set forth above. In this regard, the TCR can comprise the amino acid sequence of SEQ ID NO: 9 (variable region of anti-KRAS G12D TCR α chain): SEQ ID NO: 10 (variable region of anti-KRAS G12D TCR β chain); both SEQ ID NOs: 9 and 10: SEQ ID NO: 131 (variable region of anti-KRAS G12V TCR α chain): SEQ ID NO: 132 (variable region of anti-KRAS G12V TCR β chain); both SEQ ID NOs: 131 and 132: SEQ ID NO: 143 (variable region of anti-KRAS G12V TCR α chain); SEQ ID NO: 144 (variable region of anti-KRAS G12V TCR β chain); both SEQ ID NOs: 143 and 144: SEQ ID NO: 155 (variable region of anti-KRAS G12D TCR α chain): SEQ ID NO: 156 (variable region of anti-KRAS G12D TCR β chain); or both SEQ ID NOs: 155 and 156. Preferably, the inventive TCR comprises the amino acid sequences of both SEQ ID NOs: 9 and 10; both SEQ ID NOs: 131 and 132; both SEQ ID NOs: 143 and 144; or both SEQ ID NOs: 155 and 156.

In an embodiment of the invention, the TCR further comprises an amino acid sequence of a constant region of a TCR. In this regard, the TCR can comprise the amino acid sequence of SEQ ID NO: 13 (constant region of anti-KRAS G12D TCR α chain). SEQ ID NO: 14 (constant region of anti-KRAS G12D TCR β chain), both SEQ ID NOs: 13 and 14; SEQ ID NO: 135 (constant region of anti-KRAS G12V TCR α chain). SEQ ID NO: 136 (constant region of anti-KRAS G12V TCR β chain), both SEQ ID NOs: 135 and 136: SEQ ID NO: 147 (constant region of anti-KRAS G12V TCR α chain). SEQ ID NO: 148 (constant region of anti-KRAS G12V TCR β chain), both SEQ ID NOs: 147 and 148: SEQ ID NO: 159 (constant region of anti-KRAS G12D TCR α chain). SEQ ID NO: 160 (constant region of anti-KRAS G12D TCR β chain), or both SEQ ID NOs: 159 and 160. Preferably, the inventive TCR comprises the amino acid sequences of both SEQ ID NOs: 13 and 14; both SEQ ID NOs: 135 and 136; both SEQ ID NOs: 147 and 148; both SEQ ID NOs: 159 and 160.

In an embodiment of the invention, the inventive TCR may comprise a combination of a variable region and a constant region. In this regard, the TCR can comprise: (a) an α chain comprising the amino acid sequences of both SEQ ID NO: 9 (variable region of a chain) and SEQ ID NO: 13 (constant region of a chain): a β chain comprising the amino acid sequences of both SEQ ID NO: 10 (variable region of β chain) and SEQ ID NO: 14 (constant region of β chain); or the amino acid sequences of all of SEQ ID NOs: 9, 10, 13, and 14; (b) an α chain comprising the amino acid sequences of both SEQ ID NO: 131 (variable region of a chain) and SEQ ID NO: 135 (constant region of a chain): a β chain comprising the amino acid sequences of both SEQ ID NO: 132 (variable region of β chain) and SEQ ID NO: 136 (constant region of β chain); or the amino acid sequences of all of SEQ ID NOs: 131, 132, 135, and 136; (c) an α chain comprising the amino acid sequences of both SEQ ID NO: 143 (variable region of a chain) and SEQ ID NO: 147 (constant region of a chain): a β chain comprising the amino acid sequences of both SEQ ID NO: 144 (variable region of β chain) and SEQ ID NO: 148 (constant region of β chain); or the amino acid sequences of all of SEQ ID NOs: 143, 144, 147, and 148; or (d) an α chain comprising the amino acid sequences of both SEQ ID NO: 155 (variable region of a chain) and SEQ ID NO: 159 (constant region of a chain): a β chain comprising the amino acid sequences of both SEQ ID NO: 156 (variable region of β chain) and SEQ ID NO: 160 (constant region of β chain); or the amino acid sequences of all of SEQ ID NOs: 155, 156, 159, and 160. Preferably, the inventive TCR comprises the amino acid sequences of (a) all of SEQ ID NOs: 9, 10, 13, and 14; (b) all of SEQ ID NOs: 131, 132, 135, and 136; (c) all of SEQ ID NOs: 143, 144, 147, and 148; or (d) all of SEQ ID NOs: 155, 156, 159, and 160.

In an embodiment of the invention, the inventive TCR may comprise a combination of any of the CDR regions described herein and a constant region. In this regard, the TCR can comprise an α chain comprising: (a) the amino acid sequences of all of SEQ ID NOs: 3-5 and 13: a β chain comprising the amino acid sequences of all of SEQ ID NOs: 6-8 and 14; or the amino acid sequences of all of SEQ ID NOs: 3-8 and 13-14; (b) the amino acid sequences of all of SEQ ID NOs: 125-127 and 135: a β chain comprising the amino acid sequences of all of SEQ ID NOs: 128-130 and 136; or the amino acid sequences of all of SEQ ID NOs: 125-130 and 135-136; (c) the amino acid sequences of all of SEQ ID NOs: 137-139 and 147: a β chain comprising the amino acid sequences of all of SEQ ID NOs: 140-142 and 148; or the amino acid sequences of all of SEQ ID NOs: 137-142 and 147-148; or (d) the amino acid sequences of all of SEQ ID NOs: 149-151 and 159: a β chain comprising the amino acid sequences of all of SEQ ID NOs: 152-154 and 160; or the amino acid sequences of all of SEQ ID NOs: 149-154 and 159-160.

In an embodiment of the invention, the inventive TCR can comprise an α chain of a TCR and a β chain of a TCR. Each of the α chain and β chain of the inventive TCR can independently comprise any amino acid sequence. In this regard, the α chain of the inventive TCR can comprise the amino acid sequence of SEQ ID NO: 11 (anti-KRAS G12D TCR α chain). SEQ ID NO: 133 (anti-KRAS G12V TCR α chain), SEQ ID NO: 145 (anti-KRAS G12V TCR α chain), or SEQ ID NO: 157 (anti-KRAS G12D TCR α chain). An α chain of this type can be paired with any B chain of a TCR. In this regard, the β chain of the inventive TCR can comprise the amino acid sequence of SEQ ID NO: 12 (anti-KRAS G12D TCR β chain). SEQ ID NO: 134 (anti-KRAS G12V TCR β chain). SEQ ID NO: 146 (anti-KRAS G12V TCR β chain), or SEQ ID NO: 158 (anti-KRAS G12D TCR β chain). The inventive TCR, therefore, can comprise the amino acid sequence of SEQ ID NO: 11, SEQ ID NO: 12, SEQ ID NO: 133, SEQ ID NO: 134, SEQ ID NO: 145, SEQ ID NO: 146, SEQ ID NO: 157, SEQ ID NO: 158, both SEQ ID NOs: 11 and 12, both SEQ ID NOs: 133 and 134, both SEQ ID NOs: 145 and 146, or both SEQ ID NOs: 157 and 158. Preferably, the inventive TCR comprises the amino acid sequences of both SEQ ID NOs: 11 and 12, both SEQ ID NOs: 133 and 134, both SEQ ID NOs: 145 and 146, or both SEQ ID NOs: 157 and 158.

In an embodiment of the invention, the inventive TCRs recognize mutated target, e.g., mutated KRAS, either (i) in the presence of CD4 and the absence of CD8 or (ii) in the presence of CD8 and the absence of CD4. In a preferred embodiment, a TCR comprising the amino acid sequences of (i) SEQ ID NOs: 125-130; (ii) SEQ ID NOs: 131-132; or (iii) SEQ ID NOs: 133 and 134 recognizes mutated target, e.g., mutated KRAS, either (i) in the presence of CD4 and the absence of CD8 or (ii) in the presence of CD8 and the absence of CD4. Accordingly, these inventive TCRs may, advantageously recognize mutated target, e.g., mutated KRAS, when expressed by either CD4+ or CD8+ cells.

In an embodiment of the invention, the TCR is a murine TCR. As used herein, the term “murine.” when referring to a TCR or any component of a TCR described herein (e.g., complementarity determining region (CDR), variable region, constant region, α chain, and/or β chain), means a TCR (or component thereof) which is derived from a mouse, i.e., a TCR (or component thereof) that originated from or was, at one time, expressed by a mouse T cell. In an embodiment of the invention, a TCR comprising (i) all of SEQ ID NOs: 3-8; (ii) SEQ ID NOs: 9 and 10; (iii) SEQ ID NOs: 11 and 12; (iv) all of SEQ ID NOs: 3-8 and 13-14; (v) all of SEQ ID NOs: 9, 10, 13, and 14; (vi) all of SEQ ID NOs: 125-130; (vii) SEQ ID NOs: 131 and 132; (viii) SEQ ID NOs: 133 and 134; (ix) all of SEQ ID NOs: 125-130 and 135-136; (x) all of SEQ ID NOs: 131, 132, 135, and 136; (xi) all of SEQ ID NOs: 137-142; (xii) SEQ ID NOs: 143 and 144; (xiii) SEQ ID NOs: 145 and 146; (xiv) all of SEQ ID NOs: 137-142 and 147-148; (xv) all of SEQ ID NOs: 143, 144, 147, and 148; (xvi) all of SEQ ID NOs: 149-154; (xvii) SEQ ID NOs: 155 and 156; (xviii) SEQ ID NOs: 157 and 158; (xix) all of SEQ ID NOs: 149-154 and 159-160; or (xx) all of SEQ ID NOs: 155, 156, 159, and 160 is a murine TCR.

Included in the scope of the invention are functional variants of the inventive TCRs described herein. The term “functional variant,” as used herein, refers to a TCR, polypeptide, or protein having substantial or significant sequence identity or similarity to a parent TCR, polypeptide, or protein, which functional variant retains the biological activity of the TCR, polypeptide, or protein of which it is a variant. Functional variants encompass, for example, those variants of the TCR, polypeptide, or protein described herein (the parent TCR, polypeptide, or protein) that retain the ability to specifically bind to mutated target, e.g., mutated KRAS for which the parent TCR has antigenic specificity or to which the parent polypeptide or protein specifically binds, to a similar extent, the same extent, or to a higher extent, as the parent TCR, polypeptide, or protein. In reference to the parent TCR, polypeptide, or protein, the functional variant can, for instance, be at least about 30%, 50%, 75%, 80%, 90%, 95%, 96%, 97%, 98%, 99% or more identical in amino acid sequence to the parent TCR, polypeptide, or protein.

The functional variant can, for example, comprise the amino acid sequence of the parent TCR, polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein. In an embodiment of the invention, the functional variant is a substituted TCR, polypeptide, or protein comprising (i) the substituted CDR3α, variable region of the α chain, or full-length α chain amino acid sequence of any one of SEQ ID NOs: 46-56 and 207, 70-80 and 208, and 94-104 and 209, respectively; (ii) the substituted CDR3B, variable region of the β chain, or full-length β chain amino acid sequence of any one of SEQ ID NOs: 57-69, 81-93, and 105-117, respectively; or (iii) a pair of any one of the amino acid sequences of (i) in combination with any one of the amino acid sequences of (ii).

For example, in an embodiment of the invention, a substituted TCR, polypeptide, or protein may comprise one or both of (a) a substituted CDR3α amino acid sequence of any one of SEQ ID NOs: 46-56 and 207 (Table I) and (b) a substituted CDR3β amino acid sequence of any one of SEQ ID NOs: 57-69 (Table II). An embodiment of the invention provides a TCR, polypeptide, or protein having any one or more of the native, unsubstituted CDR1α, CDR2α, CDR1β, CDR2β, and CDR3β amino acid sequences described herein with respect to other aspects of the invention in combination with any one of the substituted CDR3α amino acid sequences of SEQ ID NOs: 46-56 and 207. In this regard, an embodiment of the invention provides a substituted TCR comprising the amino acid sequences of all of SEQ ID NOs: 149-150, 207, and 152-154. Another embodiment of the invention provides a TCR, polypeptide, or protein having any one or more of the native, unsubstituted CDR1α, CDR2α, CDR3α, CDR1β, and CDR2β amino acid sequences described herein with respect to other aspects of the invention in combination with any one of the substituted CDR3β amino acid sequences of SEQ ID NOs: 57-69.

TABLE I

| | |
|---|---|
| Substituted | CXLRGNAGAKLTF |
| CDR3 α- | Wherein X is arginine, asparagine, asparatic acid, cysteine, glutamic acid, |
| version 1 | glutamine, glycine, histidine, isoleucine, leucine, lysine, methionine, phenylalanine, |
| | proline, serine, threonine, tryptophan, tyrosine, or valine (SEQ ID NO: 46) |
| Substituted | CAXRGNAGAKLTF |
| CDR3 α- | Wherein X is alanine, arginine, asparagine, asparatic acid, cysteine, glutamic acid, |
| version 2 | glutamine, glycine, histidine, isoleucine, lysine, methionine, phenylalanine, proline, |
| | serine, threonine, tryptophan, tyrosine, or valine (SEQ ID NO: 47) |
| Substituted | CALXGNAGAKLTF |
| CDR3 α- | Wherein X is alanine, asparagine, asparatic acid, cysteine, glutamic acid, |
| version 3 | glutamine, glycine, histidine, isoleucine, leucine, lysine, methionine, phenylalanine, |
| | proline, serine, threonine, tryptophan, tyrosine, or valine (SEQ ID NO: 48) |
| Substituted | CALRXNAGAKLTF |
| CDR3 α- | Wherein X is alanine, arginine, asparagine, asparatic acid, cysteine, glutamic acid, |
| version 4 | glutamine, histidine, isoleucine, leucine, lysine, methionine, phenylalanine, proline, |
| | serine, threonine, tryptophan, tyrosine, or valine (SEQ ID NO: 49) |
| Substituted | CALRGXAGAKLTF |
| CDR3 α- | Wherein X is alanine, arginine, asparatic acid, cysteine, glutamic acid, glutamine, |
| version 5 | glycine, histidine, isoleucine, leucine, lysine, methionine, phenylalanine, proline, |
| | serine, threonine, tryptophan, tyrosine, or valine (SEQ ID NO: 50) |
| Substituted | CALRGNXGAKLTF |
| CDR3 α- | Wherein X is arginine, asparagine, asparatic acid, cysteine, glutamic acid, |
| version 6 | glutamine, glycine, histidine, isoleucine, leucine, lysine, methionine, phenylalanine, |
| | proline, serine, threonine, tryptophan, tyrosine, or valine (SEQ ID NO: 51) |
| Substituted | CALRGNAXAKLTF |
| CDR3 α- | Wherein X is alanine, arginine, asparagine, asparatic acid, cysteine, glutamic acid, |
| version 7 | glutamine, histidine, isoleucine, leucine, lysine, methionine, phenylalanine, proline, |
| | serine, threonine, tryptophan, tyrosine, or valine (SEQ ID NO: 52) |
| Substituted | CALRGNAGXKLTF |
| CDR3 α- | Wherein X is arginine, asparagine, asparatic acid, cysteine, glutamic acid, |
| version 8 | glutamine, glycine, histidine, isoleucine, leucine, lysine, methionine, phenylalanine, |
| | proline, serine, threonine, tryptophan, tyrosine, or valine (SEQ ID NO: 53) |
| Substituted | CALRGNAGAXLTF |
| CDR3 α- | Wherein X is alanine, arginine, asparagine, asparatic acid, cysteine, glutamic acid, |
| version 9 | glutamine, glycine, histidine, isoleucine, leucine, methionine, phenylalanine, proline, |
| | serine, threonine, tryptophan, tyrosine, or valine (SEQ ID NO: 54) |
| Substituted | CALRGNAGAKXTF |
| CDR3 α- | Wherein X is alanine, arginine, asparagine, asparatic acid, cysteine, glutamic acid, |
| version 10 | glutamine, glycine, histidine, isoleucine, lysine, methionine, phenylalanine, proline, |
| | serine, threonine, tryptophan, tyrosine, or valine (SEQ ID NO: 55) |
| Substituted | CALRGNAGAKLXF |
| CDR3 α- | Wherein X is alanine, arginine, asparagine, asparatic acid, cysteine, glutamic acid, |
| version 11 | glutamine, glycine, histidine, isoleucine, leucine, lysine, methionine, phenylalanine, |
| | proline, serine, tryptophan, tyrosine, or valine (SEQ ID NO: 56) |
| Substituted | CAADSSNTXYQNFYF |
| CDR3 α- | Wherein X is alanine, arginine, asparagine, asparatic acid, cysteine, glutamic acid, |
| version 12 | glutamine, histidine, isoleucine, leucine, lysine, methionine, phenylalanine, proline, |
| | serine, threonine, tryptophan, tyrosine, or valine (SEQ ID NO: 207). |
| | In a preferred embodiment, X ia alanine in SEQ ID NO: 207. |

TABLE II

| | |
|---|---|
| Substituted | CXSSSRDWSAETLYF |
| CDR3 β- | Wherein X is arginine, asparagine, asparatic acid, cysteine, glutamic acid, |
| version 1 | glutamine, glycine, histidine, isoleucine, leucine, lysine, methionine, phenylalanine, |
| | proline, serine, threonine, tryptophan, tyrosine, or valine (SEQ ID NO: 57) |
| Substituted | CAXSSRDWSAETLYF |
| CDR3 β- | Wherein X is alanine, arginine, asparagine, asparatic acid, cysteine, glutamic acid, |
| version 2 | glutamine, glycine, histidine, isoleucine, leucine, lysine, methionine, phenylalanine, |
| | proline, threonine, tryptophan, tyrosine, or valine (SEQ ID NO: 58) |
| Substituted | CASXSRDWSAETLYF |
| CDR3 β- | Wherein X is alanine, arginine, asparagine, asparatic acid, cysteine, glutamic acid, |
| version 3 | glutamine, glycine, histidine, isoleucine, leucine, lysine, methionine, phenylalanine, |
| | proline, threonine, tryptophan, tyrosine, or valine (SEQ ID NO: 59) |
| Substituted | CASSXRDWSAETLYF |
| CDR3 β- | Wherein X is alanine, arginine, asparagine, asparatic acid, cysteine, glutamic acid, |
| version 4 | glutamine, glycine, histidine, isoleucine, leucine, lysine, methionine, phenylalanine, |
| | proline, threonine, tryptophan, tyrosine, or valine (SEQ ID NO: 60) |
| Substituted | CASSSXDWSAETLYF |
| CDR3 β- | Wherein X is alanine, asparagine, asparatic acid, cysteine, glutamic acid, |
| version 5 | glutamine, glycine, histidine, isoleucine, leucine, lysine, methionine, phenylalanine, |
| | proline, serine, threonine, tryptophan, tyrosine, or valine (SEQ ID NO: 61) |
| Substituted | CASSSRXWSAETLYF |
| CDR3 β- | Wherein X is alanine, arginine, asparagine, cysteine, glutamic acid, glutamine, |
| version 6 | glycine, histidine, isoleucine, leucine, lysine, methionine, phenylalanine, proline, |
| | serine, threonine, tryptophan, tyrosine, or valine (SEQ ID NO: 62) |
| Substituted | CASSSRDXSAETLYF |
| CDR3 β- | Wherein X is alanine, arginine, asparagine, asparatic acid, cysteine, glutamic acid, |
| version 7 | glutamine, glycine, histidine, isoleucine, leucine, lysine, methionine, phenylalanine, |
| | proline, serine, threonine, tyrosine, or valine (SEQ ID NO: 63) |
| Substituted | CASSSRDWXAETLYF |
| CDR3 β- | Wherein X is alanine, arginine, asparagine, asparatic acid, cysteine, glutamic acid, |
| version 8 | glutamine, glycine, histidine, isoleucine, leucine, lysine, methionine, phenylalanine, |
| | proline, threonine, tryptophan, tyrosine, or valine (SEQ ID NO: 64) |
| Substituted | CASSSRDWSXETLYF |
| CDR3 β- | Wherein X is arginine, asparagine, asparatic acid, cysteine, glutamic acid, |
| version 9 | glutamine, glycine, histidine, isoleucine, leucine, lysine, methionine, phenylalanine, |
| | proline, serine, threonine, tryptophan, tyrosine, or valine (SEQ ID NO: 65) |
| Substituted | CASSSRDWSAXTLYF |
| CDR3 β- | Wherein X is alanine, arginine, asparagine, asparatic acid, cysteine, glutamine, |
| version 10 | glycine, histidine, isoleucine, leucine, lysine, methionine, phenylalanine, proline, |
| | serine, threonine, tryptophan, tyrosine, or valine (SEQ ID NO: 66) |
| Substituted | CASSSRDWSAEXLYF |
| CDR3 β- | Wherein X is alanine, arginine, asparagine, asparatic acid, cysteine, glutamic acid, |
| version 11 | glutamine, glycine, histidine, isoleucine, leucine, lysine, methionine, phenylalanine, |
| | proline, serine, tryptophan, tyrosine, or valine (SEQ ID NO: 67) |
| Substituted | CASSSRDWSAETXYF |
| CDR3 β- | Wherein X is alanine, arginine, asparagine, asparatic acid, cysteine, glutamic acid, |
| version 12 | glutamine, glycine, histidine, isoleucine, lysine, methionine, phenylalanine, proline, |
| | serine, threonine, tryptophan, tyrosine, or valine (SEQ ID NO: 68) |
| Substituted | CASSSRDWSAETLXF |
| CDR β- | Wherein X is alanine, arginine, asparagine, asparatic acid, cysteine, glutamic acid, |
| version 13 | glutamine, glycine, histidine, isoleucine, leucine, lysine, methionine, phenylalanine, |
| | proline, serine, threonine, tryptophan, or valine (SEQ ID NO: 69) |

In an embodiment of the invention, each of the substituted CDR3α amino acid sequences of SEQ ID NOs: 46-56 does not comprise the native, unsubstituted CDR3α amino acid sequence of SEQ ID NO: 5. In an embodiment of the invention, the substituted CDR3α amino acid sequence of SEQ ID NO: 207 does not comprise the native, unsubstituted CDR3α amino acid sequence of SEQ ID NO: 151. Similarly, in an embodiment of the invention, each of the substituted CDR3β amino acid sequences of SEQ ID NOs: 57-69 does not comprise the native, unsubstituted CDR3β amino acid sequence of SEQ ID NO: 8.

An embodiment of the invention provides a substituted TCR, polypeptide, or protein comprising one or both of (i) a substituted variable region of an α chain comprising the amino acid sequence of any one of SEQ ID NOs: 70-80 and 208 (Table III) and (ii) a substituted variable region of a β chain comprising the amino acid sequence of any one of SEQ ID NOs: 81-93 (Table IV). An embodiment of the invention provides a TCR, polypeptide, or protein having any of the native, unsubstituted variable regions of the β chain described herein with respect to other aspects of the invention in combination with any one of the substituted variable region α chain amino acid sequences of SEQ ID NOs: 70-80 and 208. Another embodiment of the invention provides a TCR, polypeptide, or protein having any of the native, unsubstituted variable regions of the α chain described herein with respect to other aspects of the invention in combination with any one of the substituted variable region β chain amino acid sequences of SEQ ID NOs: 81-93.

TABLE III

| | |
|---|---|
| Substituted | SEQ ID NO: 70, wherein X is arginine, asparagine, |
| variable | asparatic acid, cysteine, glutamic acid, glutamine, |
| region α- | glycine, histidine, isoleucine, leucine, lysine, |
| version 1 | methionine, phenylalanine, proline, serine, |
| | threonine, tryptophan, tyrosine, or valine |
| Substituted | SEQ ID NO: 71, wherein X is alanine, arginine, |
| variable | asparagine, asparatic acid, cysteine, glutamic |
| region α- | acid, glutamine, glycine, histidine, isoleucine, |
| version 2 | lysine, methionine, phenylalanine, proline, serine, |
| | threonine, tryptophan, tyrosine, or valine |
| Substituted | SEQ ID NO: 72, wherein X is alanine, asparagine, |
| variable | asparatic acid, cysteine, glutamic acid, glutamine, |
| region α- | glycine, histidine, isoleucine, leucine, lysine, |
| version 3 | methionine, phenylalanine, proline, serine, |
| | threonine, tryptophan, tyrosine, or valine |
| Substituted | SEQ ID NO: 73, wherein X is alanine, arginine, |
| variable | asparagine, asparatic acid, cysteine, glutamic |
| region α- | acid, glutamine, histidine, isoleucine, leucine, |
| version 4 | lysine, methionine, phenylalanine, proline, serine, |
| | threonine, tryptophan, tyrosine, or valine |
| Substituted | SEQ ID NO: 74, wherein X is alanine, arginine, |
| variable | asparatic acid, cysteine, glutamic acid, glutamine, |
| region α- | glycine, histidine, isoleucine, leucine, lysine, |
| version 5 | methionine, phenylalanine, proline, serine, |
| | threonine, tryptophan, tyrosine, or valine |
| Substituted | SEQ ID NO: 75, wherein X is arginine, asparagine, |
| variable | asparatic acid, cysteine, glutamic acid, glutamine, |
| region α- | glycine, histidine, isoleucine, leucine, lysine, |
| version 6 | methionine, phenylalanine, proline, serine, |
| | threonine, tryptophan, tyrosine, or valine |
| Substituted | SEQ ID NO: 76, wherein X is alanine, arginine, |
| variable | asparagine, asparatic acid, cysteine, glutamic acid, |
| region α- | glutamine, histidine, isoleucine, leucine, lysine, |
| version 7 | methionine, phenylalanine, proline, serine, |
| | threonine, tryptophan, tyrosine, or valine |
| Substituted | SEQ ID NO: 77, wherein X is arginine, asparagine, |
| variable | asparatic acid, cysteine, glutamic acid, glutamine, |
| region α- | glycine, histidine, isoleucine, leucine, lysine, |
| version 8 | methionine, phenylalanine, proline, serine, |
| | threonine, tryptophan, tyrosine, or valine |
| Substituted | SEQ ID NO: 78, wherein X is alanine, arginine, |
| variable | asparagine, asparatic acid, cysteine, glutamic |
| region α- | acid, glutamine, glycine, histidine, isoleucine, |
| version 9 | leucine, methionine, phenylalanine, proline, |
| | serine, threonine, tryptophan, tyrosine, or valine |
| Substituted | SEQ ID NO: 79, wherein X is alanine, arginine, |
| variable | asparagine, asparatic acid, cysteine, glutamic |
| region α- | acid, glutamine, glycine, histidine, isoleucine, |
| version 10 | lysine, methionine, phenylalanine, proline, serine, |
| | threonine, tryptophan, tyrosine, or valine |
| Substituted | SEQ ID NO: 80, wherein X is alanine, arginine, |
| variable | asparagine, asparatic acid, cysteine, glutamic |
| region α- | acid, glutamine, glycine, histidine, isoleucine, |
| version 11 | leucine, lysine, methionine, phenylalanine, |
| | proline, serine, tryptophan, tyrosine, or valine |
| Substituted | SEQ ID NO: 208, Wherein X is alanine, arginine, |
| variable | asparagine, asparatic acid, cysteine, glutamic |
| region α- | acid, glutamine, histidine, isoleucine, leucine, |
| version 12 | lysine, methionine, phenylalanine, proline, |
| | serine, threonine, tryptophan, tyrosine, or valine. |
| | In a preferred embodiment, X is alanine in |
| | SEQ ID NO: 208. |

TABLE IV

| | |
|---|---|
| Substituted | SEQ ID NO: 81, wherein X is arginine, asparagine, |
| variable | asparatic acid, cysteine, glutamic acid, glutamine, |
| region β- | glycine, histidine, isoleucine, leucine, lysine, |
| version 1 | methionine, phenylalanine, proline, serine, |
| | threonine, tryptophan, tyrosine, or valine |
| Substituted | SEQ ID NO: 82, wherein X is alanine, arginine, |
| variable | asparagine, asparatic acid, cysteine, glutamic acid, |
| region β- | glutamine, glycine, histidine, isoleucine, leucine, |
| version 2 | lysine, methionine, phenylalanine, proline, |
| | threonine, tryptophan, tyrosine, or valine |
| Substituted | SEQ ID NO: 83, wherein X is alanine, arginine, |
| variable | asparagine, asparatic acid, cysteine, glutamic acid, |
| region β- | glutamine, glycine, histidine, isoleucine, leucine, |
| version 3 | lysine, methionine, phenylalanine, proline, |
| | threonine, tryptophan, tyrosine, or valine |
| Substituted | SEQ ID NO: 84, wherein X is alanine, arginine, |
| variable | asparagine, asparatic acid, cysteine, glutamic acid, |
| region β- | glutamine, glycine, histidine, isoleucine, leucine, |
| version 4 | lysine, methionine, phenylalanine, proline, |
| | threonine, tryptophan, tyrosine, or valine |
| Substituted | SEQ ID NO: 85, wherein X is alanine, asparagine, |
| variable | asparatic acid, cysteine, glutamic acid, glutamine, |
| region β- | glycine, histidine, isoleucine, leucine, lysine, |
| version 5 | methionine, phenylalanine, proline, serine, |
| | threonine, tryptophan, tyrosine, or valine |
| Substituted | SEQ ID NO: 86, wherein X is alanine, arginine, |
| variable | asparagine, cysteine, glutamic acid, glutamine, |
| region β- | glycine, histidine, isoleucine, leucine, lysine, |
| version 6 | methionine, phenylalanine, proline, serine, |
| | threonine, tryptophan, tyrosine, or valine |
| Substituted | SEQ ID NO: 87, wherein X is alanine, arginine, |
| variable | asparagine, asparatic acid, cysteine, glutamic acid, |
| region β- | glutamine, glycine, histidine, isoleucine, leucine, |
| version 7 | lysine, methionine, phenylalanine, proline, |
| | serine, threonine, tyrosine, or valine |
| Substituted | SEQ ID NO: 88, wherein X is alanine, arginine, |
| variable | asparagine, asparatic acid, cysteine, glutamic |
| region β- | acid, glutamne, glycine, histidine, isoleucine, |
| version 8 | leucine, lysine, methionine, phenylalanine, |
| | proline, threonine, tryptophan, tyrosine, or valine |
| Substituted | SEQ ID NO: 89, wherein X is arginine, asparagine, |
| variable | asparatic acid, cysteine, glutamic acid, glutamine, |
| region β- | glycine, histidine, isoleucine, leucine, lysine, |
| version 9 | methionine, phenylalanine, proline, serine, |
| | threonine, tryptophan, tyrosine, or valine |
| Substituted | SEQ ID NO: 90, wherein X is alanine, arginine, |
| variable | asparagine, asparatic acid, cysteine, glutamine, |
| region β- | glycine, histidine, isoleucine, leucine, lysine, |
| version 10 | methionine, phenylalanine, proline, serine, |
| | threonine, tryptophan, tyrosine, or valine |
| Substituted | SEQ ID NO: 91, wherein X is alanine, arginine, |
| variable | asparagine, asparatic acid, cysteine, glutamic acid, |
| region β- | glutamine, glycine, histidine, isoleucine, leucine, |
| version 11 | lysine, methionine, phenylalanine, proline, |
| | serine, tryptophan, tyrosine, or valine |
| Substituted | SEQ ID NO: 92, wherein X is alanine, arginine, |
| variable | asparagine, asparatic acid, cysteine, glutamic acid, |
| region β- | glutamine, glycine, histidine, isoleucine, lysine, |
| version 12 | methionine, phenylalanine, proline, serine, |
| | threonine, tryptophan, tyrosine, or valine |
| Substituted | SEQ ID NO: 93, wherein X is alanine, arginine, |
| variable | asparagine, asparatic acid, cysteine, glutamic acid, |
| region β- | glutamine, glycine, histidine, isoleucine, leucine, |
| version 13 | lysine, methionine, phenylalanine, proline, |
| | serine, threonine, tryptophan, or valine |

In an embodiment of the invention, each of the substituted variable region α chain amino acid sequences of SEQ ID NOs: 70-80 does not comprise the native, unsubstituted variable region α chain amino acid sequence of SEQ ID NO: 9. In an embodiment of the invention, the substituted variable region α chain amino acid sequence of SEQ ID NO: 208 does not comprise the native, unsubstituted variable region α chain amino acid sequence of SEQ ID NO: 155. Similarly, in an embodiment of the invention, each of the substituted variable region β chain amino acid sequences of SEQ ID NOs: 81-93 does not comprise the native, unsubstituted variable region β chain amino acid sequence of SEQ ID NO: 10.

An embodiment of the invention provides a substituted TCR, polypeptide, or protein comprising one or both of (i) a substituted full length α chain comprising the amino acid sequence of any one of SEQ ID NOs: 94-104 and 209 (Table V) and (ii) a substituted full length β chain comprising the amino acid sequence of any one of SEQ ID NOs: 105-117 (Table VI). An embodiment of the invention provides a TCR, polypeptide, or protein having any of the native, unsubstituted full-length β chain sequences described herein with respect to other aspects of the invention in combination with any one of the substituted full length α chain amino acid sequences of SEQ ID NOs: 94-104 and 209. Another embodiment of the invention provides a TCR, polypeptide, or protein having any of the native, unsubstituted full-length α chains described herein with respect to other aspects of the invention in combination with any one of the substituted full-length β chain sequences of SEQ ID NOS: 105-117.

TABLE V

| | |
|---|---|
| Substituted | SEQ ID NO: 94, wherein X is arginine, glutamic |
| full length | asparagine, asparatic acid, cysteine, acid, |
| α chain- | glutamine, glycine, histidine, isoleucine, leucine, |
| version 1 | lysine, methionine, phenylalanine, proline, serine, |
| | threonine, tryptophan, tyrosine, or valine |
| Substituted | SEQ ID NO: 95, wherein X is alanine, arginine, |
| full length | asparagine, asparatic acid, cysteine, glutamic |
| α chain- | acid, glutamine, glycine, histidine, isoleucine, |
| version 2 | lysine, methionine, phenylalanine, proline, serine, |
| | threonine, tryptophan, tyrosine, or valine |
| Substituted | SEQ ID NO: 96, wherein X is alanine, asparagine, |
| full length | asparatic acid, cysteine, glutamic acid, glutamine, |
| α chain- | glycine, histidine, isoleucine, leucine, lysine, |
| version 3 | methionine, phenylalanine, proline, serine, |
| | threonine, tryptophan, tyrosine, or valine |
| Substituted | SEQ ID NO: 97, wherein X is alanine, arginine, |
| full length | asparagine, asparatic acid, cysteine, glutamic acid, |
| α chain- | glutamine, histidine, isoleucine, leucine, lysine, |
| version 4 | methionine, phenylalanine, proline, serine, |
| | threonine, tryptophan, tyrosine, or valine |
| Substituted | SEQ ID NO: 98, wherein X is alanine, arginine, |
| full length | asparatic acid, cysteine, glutamic acid, glutamine, |
| α chain- | glycine, histidine, isoleucine, leucine, lysine, |
| version 5 | methionine, phenylalanine, proline, serine, |
| | threonine, tryptophan, tyrosine, or valine |
| Substituted | SEQ ID NO: 99, wherein X is arginine, asparagine, |
| full length | asparatic acid, cysteine, glutamic acid, glutamine, |
| α chain- | glycine, histidine, isoleucine, leucine, lysine, |
| version 6 | methionine, phenylalanine, proline, serine, |
| | threonine, tryptophan, tyrosine, or valine |
| Substituted | SEQ ID NO: 100, wherein X is alanine, arginine, |
| full length | asparagine, asparatic acid, cysteine, glutamic acid, |
| α chain- | glutamine, histidine, isoleucine, leucine, lysine, |
| version 7 | methionine, phenylalanine, proline, serine, |
| | threonine, tryptophan, tyrosine, or valine |
| Substituted | SEQ ID NO: 101, wherein X is arginine, asparagine, |
| full length | asparatic acid, cysteine, glutamic acid, glutamine, |
| α chain- | glycine, histidine, isoleucine, leucine, lysine, |
| version 8 | methionine, phenylalanine, proline, serine, |
| | threonine, tryptophan, tyrosine, or valine |
| Substituted | SEQ ID NO: 102, wherein X is alanine, arginine, |
| full length | asparagine, asparatic acid, cysteine, glutamic acid, |
| α chain- | glutamine, glycine, histidine, isoleucine, leucine, |
| version 9 | methionine, phenylalanine, proline, serine, |
| | threonine, tryptophan, tyrosine, or valine |
| Substituted | SEQ ID NO: 103, wherein X is alanine, arginine, |
| full length | asparagine, asparatic acid, cysteine, glutamic acid, |
| α chain- | glutamine, glycine, histidine, isoleucine, lysine, |
| version 10 | methionine, phenylalanine, proline, serine, |
| | threonine, tryptophan, tyrosine, or valine |
| Substituted | SEQ ID NO: 104, wherein X is alanine, arginine, |
| full length | asparagine, asparatic acid, cysteine, glutamic acid, |
| α chain- | glutamine, glycine, histidine, isoleucine, leucine, |
| version 11 | lysine, methionine, phenylalanine, proline, |
| | serine, tryptophan, tyrosine, or valine |
| Substituted | SEQ ID NO: 209, Wherein X is alanine, arginine, |
| full length | asparagine, asparatic acid, cysteine, glutamic acid, |
| α chain- | glutamine, histidine, isoleucine, leucine, lysine, |
| version 12 | methionine, phenylalanine, proline, serine, |
| | threonine, tryptophan, tyrosine, or valine. |
| | In a preferred embodiment, X is alanine in |
| | SEQ ID NO: 209. |

TABLE VI

| | |
|---|---|
| Substituted | SEQ ID NO: 105, wherein X is arginine, asparagine, |
| full length | asparatic acid, cysteine,glutamic acid, glutamine, |
| β chain- | glycine, histidine, isoleucine, leucine, lysine, |
| version 1 | methionine, phenylalanine, proline, serine, |
| | threonine, tryptophan, tyrosine, or valine |
| Substituted | SEQ ID NO: 106, wherein X is alanine, arginine, |
| full length | asparagine, asparatic acid, cysteine, glutamic acid, |
| β chain- | glutamine, glycine, histidine, isoleucine, leucine, |
| version 2 | lysine, methionine, phenylalanine, proline, |
| | threonine, tryptophan, tyrosine, or valine |
| Substituted | SEQ ID NO: 107, wherein X is alanine, arginine, |
| full length | asparagine, asparatic acid, cysteine, glutamic acid, |
| β chain- | glutamine, glycine, histidine, isoleucine, leucine, |
| version 3 | lysine, methionine, phenylalanine, proline, |
| | threonine, tryptophan, tyrosine, or valine |
| Substituted | SEQ ID NO: 108, wherein X is alanine, arginine, |
| full length | asparagine, asparatic acid, cysteine, glutamic acid, |
| β chain- | glutamine, glycine, histidine, isoleucine, leucine, |
| version 4 | lysine, methionine, phenylalanine, proline, |
| | threonine, tryptophan, tyrosine, or valine |
| Substituted | SEQ ID NO: 109, wherein X is alanine, asparagine, |
| full length | asparatic acid, cysteine, glutamic acid, glutamine, |
| β chain- | glycine, histidine, isoleucine, leucine, lysine, |
| version 5 | methionine, phenylalanine, proline, serine, |
| | threonine, tryptophan, tyrosine, or valine |
| Substituted | SEQ ID NO: 110, wherein X is alanine, arginine, |
| full length | asparagine, cysteine, glutamic acid, glutamine, |
| β chain- | glycine, histidine, isoleucine, leucine, lysine, |
| version 6 | methionine, phenylalanine, proline, serine, |
| | threonine, tryptophan, tyrosine, or valine |
| Substituted | SEQ ID NO: 111, wherein X is alanine, arginine, |
| full length | asparagine, asparatic acid, cysteine, glutamic acid, |
| β chain- | glutamine, glycine, histidine, isoleucine, leucine, |
| version 7 | lysine, methionine, phenylalanine, proline, serine, |
| | threonine, tyrosine, or valine |
| Substituted | SEQ ID NO: 112, wherein X is alanine, arginine, |
| full length | asparagine, asparatic acid, cysteine, glutamic acid, |
| β chain- | glutamine, glycine, histidine, isoleucine, leucine, |
| version 8 | lysine, methionine, phenylalanine, proline, |
| | threonine, tryptophan, tyrosine, or valine |
| Substituted | SEQ ID NO: 113, wherein X is arginine, asparagine, |
| full length | asparatic acid, cysteine, glutamic acid, glutamine, |
| β chain- | glycine, histidine, isoleucine, leucine, lysine, |
| version 9 | methionine, phenylalanine, proline, serine, |
| | threonine, tryptophan, tyrosine, or valine |
| Substituted | SEQ ID NO: 114, wherein X is alanine, arginine, |
| full length | asparagine, asparatic acid, cysteine, glutamine, |
| β chain- | glycine, histidine, isoleucine, leucine, lysine, |
| version 10 | methionine, phenylalanine, proline, serine, |
| | threonine, tryptophan, tyrosine, or valine |
| Substituted | SEQ ID NO: 115, wherein X is alanine, arginine, |
| full length | asparagine, asparatic acid, cysteine, glutamic acid, |
| β chain- | glutamine, glycine, histidine, isoleucine, leucine, |
| version 11 | lysine, methionine, phenylalanine, proline, |
| | serine, tryptophan, tyrosine, or valine |
| Substituted | SEQ ID NO: 116, wherein X is alanine, arginine, |
| full length | asparagine, asparatic acid, cysteine, glutamic acid, |
| β chain- | glutamine, glycine, histidine, isoleucine, lysine, |
| version 12 | methionine, phenylalanine, proline, serine, |
| | threonine, tryptophan, tyrosine, or valine |
| Substituted | SEQ ID NO: 117, wherein X is alanine, arginine, |
| full length | asparagine, asparatic acid, cysteine, glutamic acid, |
| β chain- | glutamine, glycine, histidine, isoleucine, leucine, |
| version 13 | lysine, methionine, phenylalanine, proline, serine, |
| | threonine, tryptophan, or valine |

In an embodiment of the invention, each of the substituted full length α chain amino acid sequences of SEQ ID NOs: 94-104 does not comprise the native, unsubstituted full length α chain amino acid sequence of SEQ ID NO: 11. In an embodiment of the invention, the substituted full length α chain amino acid sequence of SEQ ID NO: 209 does not comprise the native, unsubstituted full length α chain amino acid sequence of SEQ ID NO: 157. Similarly, in an embodiment of the invention, each of the substituted full length β chain amino acid sequences of SEQ ID NOs: 105-117 does not comprise the native, unsubstituted full length β chain amino acid sequence of SEQ ID NO: 12.

The TCR, polypeptide, or protein can consist essentially of the specified amino acid sequence or sequences described herein, such that other components of the TCR, polypeptide, or protein, e.g., other amino acids, do not materially change the biological activity of the TCR, polypeptide, or protein. In this regard, the inventive TCR, polypeptide, or protein can, for example, consist essentially of the amino acid sequence of SEQ ID NO: 11, SEQ ID NO: 12, SEQ ID NO: 133, SEQ ID NO: 134, SEQ ID NO: 145, SEQ ID NO: 146, SEQ ID NO: 157, SEQ ID NO: 158, SEQ ID NO: 209, both SEQ ID NOs: 11 and 12, both SEQ ID NOs: 133 and 134, both SEQ ID NOs: 145 and 146, both SEQ ID NO: 157 and 158, or both SEQ ID NOs: 158 and 209. Also, for instance, the inventive TCRs, polypeptides, or proteins can consist essentially of the amino acid sequence(s) of SEQ ID NO: 9, SEQ ID NO: 10, SEQ ID NO: 131, SEQ ID NO: 132, SEQ ID NO: 143. SEQ ID NO: 144, SEQ ID NO: 155, SEQ ID NO: 156, SEQ ID NO: 208, both SEQ ID NOs: 9 and 10, both SEQ ID NOs: 131 and 132, both SEQ ID NO: 143 and 144, both SEQ ID NOs: 155 and 156, or both SEQ ID NOs: 156 and 208. Furthermore, the inventive TCRs, polypeptides, or proteins can consist essentially of the amino acid sequence of (a) SEQ ID NO: 3 (CDR1 of α chain). SEQ ID NO: 4 (CDR2 of α chain). SEQ ID NO: 5 (CDR3 of α chain). SEQ ID NO: 6 (CDR1 of β chain). SEQ ID NO: 7 (CDR2 of β chain). SEQ ID NO: 8 (CDR3 of β chain), or any combination thereof, e.g., SEQ ID NOs: 3-5:6-8; or 3-8; (b) SEQ ID NO: 125 (CDR1 of α chain). SEQ ID NO: 126 (CDR2 of α chain). SEQ ID NO: 127 (CDR3 of α chain). SEQ ID NO: 128 (CDR1 of β chain). SEQ ID NO: 129 (CDR2 of β chain). SEQ ID NO: 130 (CDR3 of β chain), or any combination thereof, e.g., SEQ ID NOs: 125-127:128-130; or 125-130; (c) SEQ ID NO: 137 (CDR1 of α chain). SEQ ID NO: 138 (CDR2 of α chain). SEQ ID NO: 139 (CDR3 of α chain). SEQ ID NO: 140 (CDR1 of β chain). SEQ ID NO: 141 (CDR2 of β chain). SEQ ID NO: 142 (CDR3 of β chain), or any combination thereof, e.g., SEQ ID NOS: 137-139:140-142; or 137-142; (d) SEQ ID NO: 149 (CDR1 of α chain). SEQ ID NO: 150 (CDR2 of α chain), SEQ ID NO: 151 (CDR3 of α chain), SEQ ID NO: 152 (CDR1 of β chain), SEQ ID NO: 153 (CDR2 of β chain), SEQ ID NO: 154 (CDR3 of β chain), or any combination thereof, e.g., SEQ ID NOs: 149-151:152-154; or 149-154; or (e) SEQ ID NO: 149 (CDR1 of α chain), SEQ ID NO: 150 (CDR2 of α chain), SEQ ID NO: 207 (substituted CDR3 of α chain), SEQ ID NO: 152 (CDR1 of β chain), SEQ ID NO: 153 (CDR2 of β chain), SEQ ID NO: 154 (CDR3 of β chain), or any combination thereof, e.g., SEQ ID NOs: 149-150 and 207:152-154; or 149-150, 207, and 152-154.

Also provided by the invention is a polypeptide comprising a functional portion of any of the TCRs described herein. The term “polypeptide” as used herein includes oligopeptides and refers to a single chain of amino acids connected by one or more peptide bonds.

With respect to the inventive polypeptides, the functional portion can be any portion comprising contiguous amino acids of the TCR of which it is a part, provided that the functional portion specifically binds to mutated target, e.g., mutated KRAS. The term “functional portion” when used in reference to a TCR refers to any part or fragment of the TCR of the invention, which part or fragment retains the biological activity of the TCR of which it is a part (the parent TCR). Functional portions encompass, for example, those parts of a TCR that retain the ability to specifically bind to mutated target, e.g., mutated KRAS (e.g., in an HLA-A11-dependent manner), or detect, treat, or prevent cancer, to a similar extent, the same extent, or to a higher extent, as the parent TCR. In reference to the parent TCR, the functional portion can comprise, for instance, about 10%, 25%, 30%, 50%, 68%, 80%, 90%, 95%, or more, of the parent TCR.

The functional portion can comprise additional amino acids at the amino or carboxy terminus of the portion, or at both termini, which additional amino acids are not found in the amino acid sequence of the parent TCR. Desirably, the additional amino acids do not interfere with the biological function of the functional portion, e.g., specifically binding to mutated target, e.g., mutated KRAS; and/or having the ability to detect cancer, treat or prevent cancer, etc. More desirably, the additional amino acids enhance the biological activity, as compared to the biological activity of the parent TCR or functional variant thereof.

The polypeptide can comprise a functional portion of either or both of the α and β chains of the TCRs of the invention, such as a functional portion comprising one of more of CDR1, CDR2, and CDR3 of the variable region(s) of the α chain and/or β chain of a TCR of the invention. In an embodiment of the invention, the polypeptide can comprise a functional portion comprising the amino acid sequence of (a) SEQ ID NO: 3 (CDR1 of α chain), 4 (CDR2 of α chain), 5 (CDR3 of α chain), 6 (CDR1 of β chain), 7 (CDR2 of β chain), 8 (CDR3 of β chain), or a combination thereof; (b) SEQ ID NO: 125 (CDR1 of α chain), 126 (CDR2 of α chain), 127 (CDR3 of α chain), 128 (CDR1 of β chain), 129 (CDR2 of β chain), 130 (CDR3 of β chain), or a combination thereof; (c) SEQ ID NO: 137 (CDR1 of α chain), 138 (CDR2 of α chain), 139 (CDR3 of α chain), 140 (CDR1 of β chain), 141 (CDR2 of β chain), 142 (CDR3 of β chain), or a combination thereof; (d) SEQ ID NO: 149 (CDR1 of α chain), 150 (CDR2 of α chain), 151 (CDR3 of α chain), 152 (CDR1 of β chain), 153 (CDR2 of β chain), 154 (CDR3 of β chain), or a combination thereof; or (e) SEQ ID NO: 149 (CDR1 of α chain), 150 (CDR2 of α chain), 207 (substituted CDR3 of α chain), 152 (CDR1 of β chain), 153 (CDR2 of β chain), 154 (CDR3 of β chain), or a combination thereof. Preferably, the inventive polypeptide comprises a functional portion comprising the amino acid sequences of SEQ ID NOs: 3-5:6-8:125-127:128-130:137-139:140-142:149-151:152-154; all of SEQ ID NOs: 3-8; all of SEQ ID NOs: 125-130; all of SEQ ID NOs: 137-142; all of SEQ ID NOs: 149-154; or all of SEQ ID NOs: 149-150, 207, and 152-154. More preferably, the polypeptide comprises a functional portion comprising the amino acid sequences of all of SEQ ID NOs: 3-8; all of SEQ ID NOs: 125-130; all of SEQ ID NOs: 137-142; all of SEQ ID NOs: 149-154; or all of SEQ ID NOs: 149-150, 207, and 152-154.

In an embodiment of the invention, the inventive polypeptide can comprise, for instance, the variable region of the inventive TCR or functional variant thereof comprising a combination of the CDR regions set forth above. In this regard, the polypeptide can comprise the amino acid sequence of SEQ ID NO: 9 (variable region of α chain), SEQ ID NO: 10 (variable region of β chain), SEQ ID NO: 131 (variable region of α chain), SEQ ID NO: 132 (variable region of β chain), SEQ ID NO: 143 (variable region of α chain), SEQ ID NO: 144 (variable region of β chain), SEQ ID NO: 155 (variable region of α chain), SEQ ID NO: 156 (variable region of β chain), SEQ ID NO: 208 (substituted variable region of α chain), both SEQ ID NOs: 9 and 10, both SEQ ID NOs: 131 and 132, both SEQ ID NOs: 143 and 144, both SEQ ID NO: 155 and 156, or both SEQ ID NOs: 208 and 156. Preferably, the polypeptide comprises the amino acid sequences of both SEQ ID NOs: 9 and 10, both SEQ ID NOs: 131 and 132, both SEQ ID NOs: 143 and 144, both SEQ ID NO: 155 and 156, or both SEQ ID NOs: 208 and 156.

In an embodiment of the invention, the inventive polypeptide can further comprise the constant region of the inventive TCR or functional variant thereof set forth above. In this regard, the polypeptide can comprise the amino acid sequence of SEQ ID NO: 13 (constant region of α chain). SEQ ID NO: 14 (constant region of β chain). SEQ ID NO: 135 (constant region of α chain). SEQ ID NO: 136 (constant region of β chain). SEQ ID NO: 147 (constant region of α chain). SEQ ID NO: 148 (constant region of β chain). SEQ ID NO: 159 (constant region of α chain). SEQ ID NO: 160 (constant region of β chain), both SEQ ID NOs: 13 and 14, both SEQ ID NOs: 135 and 136, both SEQ ID NOs: 147 and 148, or both SEQ ID NOs: 159 and 160. Preferably, the polypeptide comprises the amino acid sequences of both SEQ ID NOs: 13 and 14, both SEQ ID NOs: 135 and 136, both SEQ ID NOs: 147 and 148, or both SEQ ID NOs: 159 and 160.

In an embodiment of the invention, the inventive polypeptide may comprise a combination of a variable region and a constant region of the inventive TCR or functional variant thereof. In this regard, the polypeptide can comprise: (a) the amino acid sequences of both SEQ ID NO: 9 (variable region of α chain) and SEQ ID NO: 13 (constant region of α chain), both SEQ ID NO: 10 (variable region of β chain) and SEQ ID NO: 14 (constant region of β chain), or all of SEQ ID NOs: 9, 10, 13, and 14; (b) the amino acid sequences of both SEQ ID NO: 131 (variable region of α chain) and SEQ ID NO: 135 (constant region of αchain), both SEQ ID NO: 132 (variable region of β chain) and SEQ ID NO: 136 (constant region of β chain), or all of SEQ ID NOs: 131, 132, 135, and 136; (c) the amino acid sequences of both SEQ ID NO: 143 (variable region of α chain) and SEQ ID NO: 147 (constant region of α chain), both SEQ ID NO: 144 (variable region of β chain) and SEQ ID NO: 148 (constant region of β chain), or all of SEQ ID NOs: 143, 144, 147, and 148; (d) the amino acid sequences of both SEQ ID NO: 155 (variable region of α chain) and SEQ ID NO: 159 (constant region of α chain), both SEQ ID NO: 156 (variable region of β chain) and SEQ ID NO: 160 (constant region of β chain), or all of SEQ ID NOs: 155, 156, 159, and 160; or (e) the amino acid sequences of both SEQ ID NO: 208 (substituted variable region of αchain) and SEQ ID NO: 159 (constant region of α chain), both SEQ ID NO: 156 (variable region of β chain) and SEQ ID NO: 160 (constant region of β chain), or all of SEQ ID NOs: 208, 156, 159, and 160. Preferably, the polypeptide comprises the amino acid sequences of all of SEQ ID NOs: 9, 10, 13, and 14; all of SEQ ID NOs: 131, 132, 135, and 136; all of SEQ ID NOs: 143, 144, 147, and 148; all of SEQ ID NOs: 155, 156, 159, and 160; or all of SEQ ID NOs: 208, 156, 159, and 160.

In an embodiment of the invention, the inventive polypeptide may comprise a combination of any of the CDR regions described herein and a constant region of the inventive TCR. In this regard, the polypeptide can comprise the amino acid sequences of all of SEQ ID NOs: 3-5 and 13, all of SEQ ID NOs: 6-8 and 14, all of SEQ ID NOs: 3-8 and 13-14; all of SEQ ID NOs: 125-127 and 135, all of SEQ ID NOs: 128-130 and 136, all of SEQ ID NOs: 125-130 and 135-136, all of SEQ ID NOs: 137-139 and 147, all of SEQ ID NOs: 140-142 and 148, all of SEQ ID NOs: 137-142 and 147-148, all of SEQ ID NOs: 149-151 and 159, all of SEQ ID NOs: 149-150, 207, and 159, all of SEQ ID NOs: 152-154 and 160, all of SEQ ID NOs: 149-154 and 159-160, or all of SEQ ID NOs: 149-150, 207, 152-154, and 159-160. Preferably, the polypeptide comprises the amino acid sequences of all of SEQ ID NOs: 3-8 and 13-14, all of SEQ ID NOs: 125-130 and 135-136, all of SEQ ID NOs: 137-142 and 147-148, all of SEQ ID NOs: 149-154 and 159-160, or all of SEQ ID NOs: 149-150, 207, 152-154, and 159-160.

In an embodiment of the invention, the inventive polypeptide can comprise the entire length of an α or β chain of the TCR described herein. In this regard, the inventive polypeptide can comprise the amino acid sequence of SEQ ID NO: 11, SEQ ID NO: 12, SEQ ID NO: 133, SEQ ID NO: 134, SEQ ID NO: 145, SEQ ID NO: 146, SEQ ID NO: 157, SEQ ID NO: 209, SEQ ID NO: 158, both SEQ ID NOs: 11 and 12, both SEQ ID NOs: 133 and 134, both SEQ ID NOs: 145 and 146, both SEQ ID NOs: 157 and 158, or both SEQ ID NOS: 209 and 158. Preferably, the polypeptide comprises the amino acid sequences of both SEQ ID NOs: 11 and 12, both SEQ ID NOs: 133 and 134, both SEQ ID NOs: 145 and 146, both SEQ ID NOs: 157 and 158, or both SEQ ID NOs: 209 and 158.

The invention further provides a protein comprising at least one of the polypeptides described herein. By “protein” is meant a molecule comprising one or more polypeptide chains.

In an embodiment, the protein of the invention can comprise a first polypeptide chain comprising the amino acid sequences of SEQ ID NOs: 3-5 and a second polypeptide chain comprising the amino acid sequence of SEQ ID NOs: 6-8; a first polypeptide chain comprising the amino acid sequences of SEQ ID NOs: 125-127 and a second polypeptide chain comprising the amino acid sequence of SEQ ID NOs: 128-130; a first polypeptide chain comprising the amino acid sequences of SEQ ID NOs: 137-139 and a second polypeptide chain comprising the amino acid sequence of SEQ ID NOs: 140-142; a first polypeptide chain comprising the amino acid sequences of SEQ ID NOs: 149-151 and a second polypeptide chain comprising the amino acid sequence of SEQ ID NOs: 152-154; or a first polypeptide chain comprising the amino acid sequences of SEQ ID NOs: 149-150 and 207 and a second polypeptide chain comprising the amino acid sequence of SEQ ID NOS: 152-154. Alternatively or additionally, the protein of the invention can comprise a first polypeptide chain comprising the amino acid sequence of SEQ ID NO: 9 and a second polypeptide chain comprising the amino acid sequence of SEQ ID NO: 10; a first polypeptide chain comprising the amino acid sequence of SEQ ID NO: 131 and a second polypeptide chain comprising the amino acid sequence of SEQ ID NO: 132; a first polypeptide chain comprising the amino acid sequence of SEQ ID NO: 143 and a second polypeptide chain comprising the amino acid sequence of SEQ ID NO: 144; a first polypeptide chain comprising the amino acid sequence of SEQ ID NO: 155 and a second polypeptide chain comprising the amino acid sequence of SEQ ID NO: 156; or a first polypeptide chain comprising the amino acid sequence of SEQ ID NO: 208 and a second polypeptide chain comprising the amino acid sequence of SEQ ID NO: 156. The protein can, for example, comprise (a) a first polypeptide chain comprising the amino acid sequences of both SEQ ID NOs: 9 and 13 or all of SEQ ID NOs: 3-5 and 13 and a second polypeptide chain comprising the amino acid sequences of both SEQ ID NOs: 10 and 14 or all of SEQ ID NOs: 6-8 and 14; (b) a first polypeptide chain comprising the amino acid sequences of both SEQ ID NOs: 131 and 135 or all of SEQ ID NOs: 125-127 and 135 and a second polypeptide chain comprising the amino acid sequences of both SEQ ID NOs: 132 and 136 or all of SEQ ID NOs: 128-130 and 136; (c) a first polypeptide chain comprising the amino acid sequences of both SEQ ID NOs: 143 and 147 or all of SEQ ID NOs: 137-139 and 147 and a second polypeptide chain comprising the amino acid sequences of both SEQ ID NOs: 144 and 148 or all of SEQ ID NOs: 140-142 and 148; (d) a first polypeptide chain comprising the amino acid sequences of both SEQ ID NOs: 155 and 159 or all of SEQ ID NOs: 149-151 and 159 and a second polypeptide chain comprising the amino acid sequences of both SEQ ID NOs: 156 and 160 or all of SEQ ID NOs: 152-154 and 160; or (e) a first polypeptide chain comprising the amino acid sequences of both SEQ ID NOs: 208 and 159 or all of SEQ ID NOs: 149-150, 207 and 159 and a second polypeptide chain comprising the amino acid sequences of both SEQ ID NOs: 156 and 160 or all of SEQ ID NOs: 152-154 and 160. Alternatively or additionally, the protein of the invention can comprise (a) a first polypeptide chain comprising the amino acid sequence of SEQ ID NO: 11 and a second polypeptide chain comprising the amino acid sequence of SEQ ID NO: 12; (b) a first polypeptide chain comprising the amino acid sequence of SEQ ID NO: 133 and a second polypeptide chain comprising the amino acid sequence of SEQ ID NO: 134; (c) a first polypeptide chain comprising the amino acid sequence of SEQ ID NO: 145 and a second polypeptide chain comprising the amino acid sequence of SEQ ID NO: 146; (d) a first polypeptide chain comprising the amino acid sequence of SEQ ID NO: 157 and a second polypeptide chain comprising the amino acid sequence of SEQ ID NO: 158; or (e) a first polypeptide chain comprising the amino acid sequence of SEQ ID NO: 209 and a second polypeptide chain comprising the amino acid sequence of SEQ ID NO: 158. In this instance, the protein of the invention can be a TCR. Alternatively, if, for example, the protein comprises a single polypeptide chain comprising the amino acid sequences of both SEQ ID NOs: 11 and 12, both SEQ ID NOs: 133 and 134, both SEQ ID NOs: 145 and 146, both SEQ ID NOs: 157 and 158, or both SEQ ID NOs: 209 and 158, or if the first and/or second polypeptide chain(s) of the protein further comprise(s) other amino acid sequences, e.g., an amino acid sequence encoding an immunoglobulin or a portion thereof, then the inventive protein can be a fusion protein. In this regard, the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof, including, but not limited to an immunoglobulin. CD3, CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods.

In some embodiments of the invention, the TCRs, polypeptides, and proteins of the invention may be expressed as a single protein comprising a linker peptide linking the α chain and the β chain. In this regard, the TCRs, polypeptides, and proteins of the invention comprising both SEQ ID NOs: 11 and 12, both SEQ ID NOs: 133 and 134, both SEQ ID NOs: 145 and 146, both SEQ ID NOs: 157 and 158, both SEQ ID NOs: 209 and 158, both SEQ ID NO: 9 and 10, both SEQ ID NOs: 131 and 132, both SEQ ID NOs: 143 and 144, both SEQ ID NOs: 155 and 156, both SEQ ID NOs: 208 and 156, all of SEQ ID NOs: 3-8, all of SEQ ID NOs: 125-130, all of SEQ ID NOs: 137-142, all of SEQ ID NOs: 149-154, all of SEQ ID NOs: 9, 10, 13, and 14, all of SEQ ID NOs: 131, 132, 135, and 136, all of SEQ ID NOs: 143, 144, 147, and 148, all of SEQ ID NOs: 155, 156, 159, and 160, all of SEQ ID NOs: 208, 156, 159, and 160, all of SEQ ID NOs: 3-8 and 13-14, all of SEQ ID NOs: 125-130 and 135-136, all of SEQ ID NOs: 137-142 and 147-148, all of SEQ ID NOs: 149-154 and 159-160, or all of SEQ ID NOs: 149-150, 207, 152-154, and 159-160 may further comprise a linker peptide. The linker peptide may advantageously facilitate the expression of a recombinant TCR, polypeptide, and/or protein in a host cell. The linker peptide may comprise any suitable amino acid sequence. In an embodiment of the invention, the TCR, polypeptide, or protein comprises a self-cleaving, viral linker peptide. For example, the linker peptide may comprise SEQ ID NO: 28. Upon expression of the construct including the linker peptide by a host cell, the linker peptide may be cleaved, resulting in separated α and β chains. In an embodiment of the invention, the TCR, polypeptide, or protein may comprise an amino acid sequence comprising a full-length α chain, a full-length β chain, and a linker peptide positioned between the α and β chains (for example, the amino acid sequence of SEQ ID NO: 45 (anti-KRAS G12D TCR), SEQ ID NO: 162 (anti-KRAS G12D TCR), SEQ ID NO: 201 (anti-KRAS G12V TCR), or SEQ ID NO: 203 (anti-KRAS G12V TCR)).

The protein of the invention can be a recombinant antibody comprising at least one of the inventive polypeptides described herein. As used herein, “recombinant antibody” refers to a recombinant (e.g., genetically engineered) protein comprising at least one of the polypeptides of the invention and a polypeptide chain of an antibody, or a portion thereof. The polypeptide of an antibody, or portion thereof, can be a heavy chain, a light chain, a variable or constant region of a heavy or light chain, a single chain variable fragment (scFv), or an Fc, Fab, or F(ab)₂′ fragment of an antibody, etc. The polypeptide chain of an antibody, or portion thereof, can exist as a separate polypeptide of the recombinant antibody. Alternatively, the polypeptide chain of an antibody, or portion thereof, can exist as a polypeptide, which is expressed in frame (in tandem) with the polypeptide of the invention. The polypeptide of an antibody, or portion thereof, can be a polypeptide of any antibody or any antibody fragment, including any of the antibodies and antibody fragments described herein.

The TCRs, polypeptides, and proteins of the invention (including functional variants thereof) can be of any length, i.e., can comprise any number of amino acids, provided that the TCRs, polypeptides, or proteins (or functional variants thereof) retain their biological activity, e.g., the ability to specifically bind to mutated target, e.g., mutated KRAS; detect cancer in a mammal; or treat or prevent cancer in a mammal, etc. For example, the polypeptide can be in the range of from about 50 to about 5000 amino acids long, such as 50, 70, 75, 100, 125, 150, 175, 200, 300, 400, 500, 600, 700, 800, 900, 1000 or more amino acids in length. In this regard, the polypeptides of the invention also include oligopeptides.

The TCRs, polypeptides, and proteins of the invention of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, ox-(2-amino-2-norbornane)-carboxylic acid, α,γ-diaminobutyric acid, α,β-diaminopropionic acid, homophenylalanine, and α-tert-butylglycine.

The TCRs, polypeptides, and proteins of the invention (including functional variants thereof) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The TCR, polypeptide, and/or protein of the invention can be obtained by methods known in the art such as, for example, de novo synthesis. Also, polypeptides and proteins can be recombinantly produced using the nucleic acids described herein using standard recombinant methods. See, for instance, Green and Sambrook, Molecular Cloning: A Laboratory Manual, 4th ed., Cold Spring Harbor Press, Cold Spring Harbor, NY (2012). Alternatively, the TCRs, polypeptides, and/or proteins described herein (including functional variants thereof) can be commercially synthesized by companies, such as Synpep (Dublin, CA), Peptide Technologies Corp. (Gaithersburg. MD), and Multiple Peptide Systems (San Diego, CA). In this respect, the inventive TCRs, polypeptides, and proteins can be synthetic, recombinant, isolated, and/or purified.

Included in the scope of the invention are conjugates, e.g., bioconjugates, comprising any of the inventive TCRs, polypeptides, or proteins, nucleic acids, recombinant expression vectors, host cells, populations of host cells, and antibodies, or antigen binding portions thereof. Conjugates, as well as methods of synthesizing conjugates in general, are known in the art.

An embodiment of the invention provides a nucleic acid comprising a nucleotide sequence encoding any of the TCRs, polypeptides, or proteins described herein. “Nucleic acid.” as used herein, includes “polynucleotide.” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In an embodiment, the nucleic acid comprises complementary DNA (cDNA). It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Green and Sambrook et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxy hydroxymethyl) uracil, 5-carboxymethylaminomethyl-2-thiouridine, 5-carboxy methylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxy carboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins. CO) and Synthegen (Houston. TX).

The nucleic acid can comprise any nucleotide sequence which encodes any of the TCRs, polypeptides, or proteins described herein. In an embodiment of the invention, the nucleic acid may comprise the nucleotide sequence of (a) SEQ ID NO: 22 (CDR1 of anti-KRAS G12D TCR α chain); the nucleotide sequence of SEQ ID NO: 23 (CDR2 of anti-KRAS G12D TCR α chain); the nucleotide sequence of SEQ ID NO: 24 (CDR3 of anti-KRAS G12D TCR α chain); the nucleotide sequence of SEQ ID NO: 25 (CDR1 of anti-KRAS G12D TCR β chain); the nucleotide sequence of SEQ ID NO: 26 (CDR2 of anti-KRAS G12D TCR β chain); or the nucleotide sequence of SEQ ID NO: 27 (CDR3 of anti-KRAS G12D TCR β chain); (b) SEQ ID NO: 164 (CDR1 of anti-KRAS G12D TCR α chain); the nucleotide sequence of SEQ ID NO: 165 (CDR2 of anti-KRAS G12D TCR α chain); the nucleotide sequence of SEQ ID NO: 166 (CDR3 of anti-KRAS G12D TCR α chain); the nucleotide sequence of SEQ ID NO: 167 (CDR1 of anti-KRAS G12D TCR β chain); the nucleotide sequence of SEQ ID NO: 168 (CDR2 of anti-KRAS G12D TCR β chain); or the nucleotide sequence of SEQ ID NO: 169 (CDR3 of anti-KRAS G12D TCR β chain); (c) SEQ ID NO: 177 (CDR1 of anti-KRAS G12V TCR α chain); the nucleotide sequence of SEQ ID NO: 178 (CDR2 of anti-KRAS G12V TCR α chain); the nucleotide sequence of SEQ ID NO: 179 (CDR3 of anti-KRAS G12V TCR α chain); the nucleotide sequence of SEQ ID NO: 180 (CDR1 of anti-KRAS G12V TCR β chain); the nucleotide sequence of SEQ ID NO: 181 (CDR2 of anti-KRAS G12V TCR β chain); or the nucleotide sequence of SEQ ID NO: 182 (CDR3 of anti-KRAS G12V TCR β chain); or (d) SEQ ID NO: 189 (CDR1 of anti-KRAS G12V TCR α chain); the nucleotide sequence of SEQ ID NO: 190 (CDR2 of anti-KRAS G12V TCR α chain); the nucleotide sequence of SEQ ID NO: 191 (CDR3 of anti-KRAS G12V TCR α chain); the nucleotide sequence of SEQ ID NO: 192 (CDR1 of anti-KRAS G12V TCR β chain); the nucleotide sequence of SEQ ID NO: 193 (CDR2 of anti-KRAS G12V TCR β chain); or the nucleotide sequence of SEQ ID NO: 194 (CDR3 of anti-KRAS G12V TCR β chain). Preferably, the nucleic acid comprises the nucleotide sequences of all of SEQ ID NOs: 22-24; all of SEQ ID NOs: 25-27; all of SEQ ID NOs: 22-27; all of SEQ ID NOs: 164-166; all of SEQ ID NOs: 167-169; all of SEQ ID NOs: 164-169; all of SEQ ID NOs: 177-179; all of SEQ ID NOs: 180-182; all of SEQ ID NOs: 177-182; all of SEQ ID NOs: 189-191; all of SEQ ID NOs: 192-194; SEQ ID NOs: 189-194. In an especially preferred embodiment, the nucleic acid comprises the nucleotide sequences of all of SEQ ID NOs: 22-27; all of SEQ ID NOs: 164-169; all of SEQ ID NOs: 177-182; or all of SEQ ID NOs: 189-194. In an embodiment of the invention, the nucleic acid may comprise the nucleotide sequence of (a) SEQ ID NO: 15 (variable region of anti-KRAS G12D TCR αchain); SEQ ID NO: 16 (variable region of anti-KRAS G12D TCR β chain); or both SEQ ID NOs: 15 and 16; (b) SEQ ID NO: 170 (variable region of anti-KRAS G12D TCR α chain); SEQ ID NO: 171 (variable region of anti-KRAS G12D TCR β chain); or both SEQ ID NOs: 170 and 171; (c) SEQ ID NO: 183 (variable region of anti-KRAS G12V TCR α chain); SEQ ID NO: 184 (variable region of anti-KRAS G12V TCR β chain); or both SEQ ID NOs: 183 and 184; (d) SEQ ID NO: 195 (variable region of anti-KRAS G12V TCR α chain); SEQ ID NO: 196 (variable region of anti-KRAS G12V TCR β chain); or both SEQ ID NOs: 195 and 196. Preferably, the nucleic acid comprises the nucleotide sequences of both SEQ ID NOS: 15 and 16; both SEQ ID NOs: 170 and 171; both SEQ ID NOs: 183 and 184; or both SEQ ID NOs: 195 and 196. In another embodiment of the invention, the nucleic acid may comprise the nucleotide sequence of (a) SEQ ID NO: 17 (full-length anti-KRAS G12D TCR α chain); SEQ ID NO: 18 (full length anti-KRAS G12D TCR β chain); or both of SEQ ID NOs: 17 and 18; (b) SEQ ID NO: 172 (full-length anti-KRAS G12D TCR α chain); SEQ ID NO: 173 (full length anti-KRAS G12D TCR β chain); or both of SEQ ID NOs: 172 and 173; (c) SEQ ID NO: 185 (full-length anti-KRAS G12V TCR α chain); SEQ ID NO: 186 (full length anti-KRAS G12V TCR β chain); or both of SEQ ID NOs: 185 and 186; or (d) SEQ ID NO: 197 (full-length anti-KRAS G12V TCR α chain); SEQ ID NO: 198 (full length anti-KRAS G12V TCR β chain); or both of SEQ ID NOs: 197 and 198. Preferably, the nucleic acid comprises the nucleotide sequences of both of SEQ ID NOs: 17 and 18; both SEQ ID NOs: 172 and 173; both SEQ ID NOs: 185 and 186; or both SEQ ID NOs: 197 and 198.

In an embodiment of the invention, the nucleic acid further comprises a nucleotide sequence that encodes the constant region of a TCR α or β chain. In this regard, any of the nucleic acids described herein may further comprise the nucleotide sequence of (a) SEQ ID NO: 19 (constant region of anti-KRAS G12D TCR α chain); SEQ ID NO: 20 (constant region of anti-KRAS G12D TCR β chain); or both SEQ ID NOs: 19 and 20; (b) SEQ ID NO: 174 (constant region of anti-KRAS G12D TCR α chain); SEQ ID NO: 175 (constant region of anti-KRAS G12D TCR β chain); or both SEQ ID NOs: 174 and 175; (c) SEQ ID NO: 187 (constant region of anti-KRAS G12V TCR α chain); SEQ ID NO: 188 (constant region of anti-KRAS G12V TCR β chain); or both SEQ ID NOs: 187 and 188; or (d) SEQ ID NO: 199 (constant region of anti-KRAS G12V TCR α chain); SEQ ID NO: 200 (constant region of anti-KRAS G12V TCR β chain); or both SEQ ID NOs: 199 and 200. Preferably, the nucleic acid comprises the nucleotide sequence of both SEQ ID NOs: 15 and 19; both SEQ ID NOS: 16 and 20; all of SEQ ID NOs: 15-16 and 19-20; all of SEQ ID NOs: 22-24 and 19; all of SEQ ID NOs: 25-27 and 20; all of SEQ ID NOs: 22-27 and 19-20; both SEQ ID NO: 170 and 174; both SEQ ID NOs: 171 and 175; all of SEQ ID NOs: 170-171 and 174-175; all of SEQ ID NOs: 164-166 and 174; all of SEQ ID NOs: 167-169 and 175; all of SEQ ID NOs: 164-169 and 174-175; both of SEQ ID NOs: 183 and 187; both of SEQ ID NOs: 184 and 188; all of SEQ ID NOs: 183-184 and 187-188: SEQ ID NO: 177-179 and 187; all of SEQ ID NOs: 180-182 and 188; all of SEQ ID NOs: 177-182 and 187-188; both of SEQ ID NOs: 195 and 199; both of SEQ ID NOs: 196 and 200; all of SEQ ID NOs: 195-196 and 199-200; all of SEQ ID NOs: 189-191 and 199; all of SEQ ID NOs: 192-194 and 200; or all of SEQ ID NOs: 189-194 and 199-200. In an especially preferred embodiment, the nucleic acid comprises the nucleotide sequences of all of SEQ ID NOs: 15-16 and 19-20; all of SEQ ID NOs: 22-27 and 19-20; all of SEQ ID NOs: 170-171 and 174-175; all of SEQ ID NOs: 164-169 and 174-175; all of SEQ ID NOs: 183-184 and 187-188; all of SEQ ID NOs: 177-182 and 187-188; all of SEQ ID NOs: 195-196 and 199-200; or all of SEQ ID NOs: 189-194 and 199-200.

Any of the nucleic acids described herein may further comprise a nucleotide sequence encoding a linker peptide. The nucleotide sequence encoding the linker peptide may comprise any suitable nucleotide sequence. For example, the nucleotide sequence encoding a linker peptide may comprise the nucleotide sequence of SEQ ID NO: 44.

In an embodiment of the invention, a nucleic acid comprising the nucleotide sequence of all of SEQ ID NOs: 22-24; all of SEQ ID NOs: 25-27; all of SEQ ID NOs: 22-27; both SEQ ID NOs: 15 and 16; both SEQ ID NOs: 17 and 18; both SEQ ID NOs: 15 and 19; both SEQ ID NOs: 16 and 20; all of SEQ ID NOs: 15-16 and 19-20; all of SEQ ID NOs: 22-24 and 19; all of SEQ ID NOs: 25-27 and 20; all of SEQ ID NOs: 22-27 and 19-20; all of SEQ ID NOs: 164-169; both SEQ ID NOs: 170 and 171; both SEQ ID NOs: 172 and 173; all of SEQ ID NOs: 164-169 and 174-175; all of SEQ ID NOs: 170-171 and 174-175; all of SEQ ID NOs: 177-182; both of SEQ ID NO: 183-184; both of SEQ ID NOs: 185-186; all of SEQ ID NOs: 177-182 and 187-188; all of SEQ ID NOs: 183-184 and 187-188; all of SEQ ID NOs: 189-194; both of SEQ ID NOs: 195-196; both of SEQ ID NOs: 197-198; all of SEQ ID NOs: 189-194 and 199-200; or all of SEQ ID NOs: 195-196 and 199-200 encodes a murine TCR.

The invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10) bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions can be rendered more stringent by the addition of increasing amounts of formamide.

The invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein. In this regard, the nucleic acid may consist essentially of any of the nucleotide sequences described herein.

The nucleic acids of the invention can be incorporated into a recombinant expression vector. In this regard, the invention provides a recombinant expression vector comprising any of the nucleic acids of the invention. In an embodiment of the invention, the recombinant expression vector comprises a nucleotide sequence encoding the α chain, the β chain, and linker peptide. For example, in an embodiment, the recombinant expression vector comprises the nucleotide sequence of SEQ ID NO: 21 (encoding α and β chains SEQ ID NOs: 11 and 12 with a linker positioned between them): SEQ ID NO: 163 (encoding a and β chains SEQ ID NOs: 157 and 158 with a linker positioned between them): SEQ ID NO: 202 (encoding α and β chains SEQ ID NOs: 145 and 146 with a linker positioned between them); or SEQ ID NO: 176 (encoding α and β chains SEQ ID NOs: 133 and 134 with a linker positioned between them).

For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotide, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages does not hinder the transcription or replication of the vector.

The recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM and pMAMneo (Clontech). Preferably, the recombinant expression vector is a viral vector, e.g., a retroviral vector. In an especially preferred embodiment, the recombinant expression vector is an MSGV1 vector.

The recombinant expression vectors of the invention can be prepared using standard recombinant DNA techniques described in, for example, Green and Sambrook et al., supra. Constructs of expression vectors, which are circular or linear, can be prepared to contain a replication system functional in a prokaryotic or eukaryotic host cell. Replication systems can be derived, e.g., from ColE1, 2μ plasmid, λ, SV40, bovine papillomavirus, and the like.

Desirably, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the TCR, polypeptide, or protein, or to the nucleotide sequence which is complementary to or which hybridizes to the nucleotide sequence encoding the TCR, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent. e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine daminase, purine nucleoside phosphorylase, and nitroreductase.

Another embodiment of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5α cell. For purposes of producing a recombinant TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL) or a peripheral blood mononuclear cell (PBMC). More preferably, the host cell is a T cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. The T cell can be any type of T cell and can be of any developmental stage, including but not limited to, CD4⁺/CD8⁺ double positive T cells, CD4⁺ helper T cells, e.g., Th₁ and Th₂ cells, CD4⁺ T cells, CD8⁺ T cells (e.g., cytotoxic T cells), tumor infiltrating lymphocytes (TILs), memory T cells (e.g., central memory T cells and effector memory T cells), naïve T cells, and the like.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

In an embodiment of the invention, the numbers of cells in the population may be rapidly expanded. Expansion of the numbers of T cells can be accomplished by any of a number of methods as are known in the art as described in, for example, U.S. Pat. No. 8,034,334: U.S. Pat. No. 8,383,099; U.S. Patent Application Publication No. 2012/0244133; Dudley et al., J. Immunother., 26:332-42 (2003); and Riddell et al., J. Immunol. Methods, 128:189-201 (1990). In an embodiment, expansion of the numbers of T cells is carried out by culturing the T cells with OKT3 antibody, IL-2, and feeder PBMC (e.g., irradiated allogeneic PBMC).

The invention further provides an antibody, or antigen binding portion thereof, which specifically binds to a functional portion of any of the TCRs described herein. Preferably, the functional portion specifically binds to the cancer antigen, e.g., the functional portion comprising the amino acid sequence SEQ ID NO: 3, 125, 137, or 149 (CDR1 of α chain), SEQ ID NO: 4, 126, 138, or 150 (CDR2 of α chain), SEQ ID NO: 5, 127, 139, 151, or 207 (CDR3 of α chain), SEQ ID NO: 6, 128, 140, or 152 (CDR1 of β chain), SEQ ID NO: 7, 129, 141, or 153 (CDR2 of β chain), SEQ ID NO: 8, 130, 142, or 154 (CDR3 of β chain), SEQ ID NO: 9, 131, 143, 155, or 208 (variable region of α chain), SEQ ID NO: 10, 132, 144, or 156 (variable region of β chain), or a combination thereof, e.g., SEQ ID NOs: 3-5: SEQ ID NOs: 6-8: SEQ ID NOs: 3-8: SEQ ID NO: 9; SEQ ID NO: 10: SEQ ID NOs: 9-10: SEQ ID NOs: 125-127, SEQ ID NOs: 128-130, SEQ ID NOs: 125-130, SEQ ID NOs: 137-139, SEQ ID NOs: 140-142, SEQ ID NOs: 137-142, SEQ ID NOs: 149-151, SEQ ID NOs: 149-150 and 207, SEQ ID NOs: 152-154, SEQ ID NOs: 149-154: SEQ ID NOs: 149-150, 207, and 152-154: SEQ ID NOs: 131-132, SEQ ID NOs: 143-144, SEQ ID NOs: 155-156; SEQ ID NO: 208; or SEQ ID NOs: 208 and 156, More preferably, the functional portion comprises the amino acid sequences of SEQ ID NOs: 3-8, SEQ ID NOs: 9 and 10, SEQ ID NOs: 125-130, SEQ ID NOs: 137-142, SEQ ID NOs: 149-154, SEQ ID NOs: 149-150, 207, and 152-154, SEQ ID NOs: 131-132, SEQ ID NOs: 143-144, SEQ ID NOs: 155-156, or SEQ ID NOs: 208 and 156. In a preferred embodiment, the antibody, or antigen binding portion thereof, binds to an epitope which is formed by all 6 CDRs (CDR1-3 of the α chain and CDR1-3 of the β chain). The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody. e.g., a humanized antibody or a chimeric antibody. The antibody can be in monomeric or polymeric form. Also, the antibody can have any level of affinity or avidity for the functional portion of the inventive TCR. Desirably, the antibody is specific for the functional portion of the inventive TCR, such that there is minimal cross-reaction with other peptides or proteins.

Methods of testing antibodies for the ability to bind to any functional portion or functional variant of the inventive TCR are known in the art and include any antibody-antigen binding assay, such as, for example, radioimmunoassay (RIA), ELISA, Western blot, immunoprecipitation, and competitive inhibition assays.

Suitable methods of making antibodies are known in the art. For instance, standard hybridoma methods are described in, e.g., C. A. Janeway et al. (eds.). Immunobiology, 8^{th} Ed., Garland Publishing, New York, NY (2011)). Alternatively, other methods, such as EBV-hybridoma methods, methods of producing antibodies in non-human animals, and bacteriophage vector expression systems are known in the art.

Phage display can also be used to generate the antibody of the invention. In this regard, phage libraries encoding antigen-binding variable (V) domains of antibodies can be generated using standard molecular biology and recombinant DNA techniques (see, e.g., Green and Sambrook et al. (eds.), Molecular Cloning, A Laboratory Manual, 4^{th} Edition. Cold Spring Harbor Laboratory Press, New York (2012)). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra).

Methods for generating humanized antibodies are well known in the art. Antibodies can also be produced by transgenic mice that are transgenic for specific heavy and light chain immunoglobulin genes. Such methods are known in the art and described in, for example, Janeway et al., supra.

The invention also provides antigen binding portions of any of the antibodies described herein. The antigen binding portion can be any portion that has at least one antigen binding site, such as Fab, F(ab′)₂, dsFv, sFv, diabodies, and triabodies.

A single-chain variable region fragment (sFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Similarly, disulfide-stabilized variable region fragments (dsFv) can be prepared by recombinant DNA technology. Antibody fragments of the invention, however, are not limited to these exemplary types of antibody fragments.

Also, the antibody, or antigen binding portion thereof, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

The inventive TCRs, polypeptides, proteins, nucleic acids, recombinant expression vectors, host cells (including populations thereof), and antibodies (including antigen binding portions thereof), can be isolated and/or purified. The term “isolated” as used herein means having been removed from its natural environment. The term “purified” as used herein means having been increased in purity, wherein “purity.” is a relative term, and not to be necessarily construed as absolute purity. For example, the purity can be at least about 50%, can be greater than 60%, 70%, 80%, 90%, 95%, or can be 100%.

The inventive TCRs, polypeptides, proteins, nucleic acids, recombinant expression vectors, host cells (including populations thereof), and antibodies (including antigen binding portions thereof), all of which are collectively referred to as “inventive TCR materials” hereinafter, can be formulated into a composition, such as a pharmaceutical composition. In this regard, the invention provides a pharmaceutical composition comprising any of the TCRs, polypeptides, proteins, nucleic acids, expression vectors, host cells (including populations thereof), and antibodies (including antigen binding portions thereof) described herein, and a pharmaceutically acceptable carrier. The inventive pharmaceutical compositions containing any of the inventive TCR materials can comprise more than one inventive TCR material, e.g., a polypeptide and a nucleic acid, or two or more different TCRs. Alternatively, the pharmaceutical composition can comprise an inventive TCR material in combination with another pharmaceutically active agent(s) or drug(s), such as a chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular inventive TCR material under consideration. Such pharmaceutically acceptable carriers are well-known to those skilled in the art and are readily available to the public. It is preferred that the pharmaceutically acceptable carrier be one which has no detrimental side effects or toxicity under the conditions of use.

The choice of carrier will be determined in part by the particular inventive TCR material, as well as by the particular method used to administer the inventive TCR material. Accordingly, there are a variety of suitable formulations of the pharmaceutical composition of the invention. Suitable formulations may include any of those for oral, parenteral, subcutaneous, intravenous, intramuscular, intraarterial, intrathecal, or interperitoneal administration. More than one route can be used to administer the inventive TCR materials, and in certain instances, a particular route can provide a more immediate and more effective response than another route.

Preferably, the inventive TCR material is administered by injection, e.g., intravenously. When the inventive TCR material is a host cell expressing the inventive TCR (or functional variant thereof), the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an embodiment, the pharmaceutically acceptable carrier is supplemented with human serum albumen.

For purposes of the invention, the amount or dose (e.g., numbers of cells when the inventive TCR material is one or more cells) of the inventive TCR material administered should be sufficient to effect, e.g., a therapeutic or prophylactic response, in the subject or animal over a reasonable time frame. For example, the dose of the inventive TCR material should be sufficient to bind to a cancer antigen (e.g., mutated KRAS), or detect, treat or prevent cancer in a period of from about 2 hours or longer, e.g., 12 to 24 or more hours, from the time of administration. In certain embodiments, the time period could be even longer. The dose will be determined by the efficacy of the particular inventive TCR material and the condition of the animal (e.g., human), as well as the body weight of the animal (e.g., human) to be treated.

Many assays for determining an administered dose are known in the art. For purposes of the invention, an assay, which comprises comparing the extent to which target cells are lysed or IFN-γ is secreted by T cells expressing the inventive TCR, polypeptide, or protein upon administration of a given dose of such T cells to a mammal among a set of mammals of which is each given a different dose of the T cells, could be used to determine a starting dose to be administered to a mammal. The extent to which target cells are lysed or IFN-γ is secreted upon administration of a certain dose can be assayed by methods known in the art.

The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration, and the severity of the cancer being treated. In an embodiment in which the inventive TCR material is a population of cells, the number of cells administered per infusion may vary, e.g., from about 1×10⁶ to about 1×10¹² cells or more. In certain embodiments, fewer than 1×10⁶ cells may be administered.

One of ordinary skill in the art will readily appreciate that the inventive TCR materials of the invention can be modified in any number of ways, such that the therapeutic or prophylactic efficacy of the inventive TCR materials is increased through the modification. For instance, the inventive TCR materials can be conjugated either directly or indirectly through a bridge to a targeting moiety. The practice of conjugating compounds, e.g., inventive TCR materials, to targeting moieties is known in the art. The term “targeting moiety” as used herein, refers to any molecule or agent that specifically recognizes and binds to a cell-surface receptor, such that the targeting moiety directs the delivery of the inventive TCR materials to a population of cells on which surface the receptor is expressed. Targeting moieties include, but are not limited to, antibodies, or fragments thereof, peptides, hormones, growth factors, cytokines, and any other natural or non-natural ligands, which bind to cell surface receptors (e.g., Epithelial Growth Factor Receptor (EGFR), T cell receptor (TCR), B-cell receptor (BCR), CD28, Platelet-derived Growth Factor Receptor (PDGF), nicotinic acetylcholine receptor (nAChR), etc.). The term “bridge” as used herein, refers to any agent or molecule that links the inventive TCR materials to the targeting moiety. One of ordinary skill in the art recognizes that sites on the inventive TCR materials, which are not necessary for the function of the inventive TCR materials, are ideal sites for attaching a bridge and/or a targeting moiety, provided that the bridge and/or targeting moiety, once attached to the inventive TCR materials, do(es) not interfere with the function of the inventive TCR materials, i.e., the ability to bind to mutated target, e.g., mutated KRAS or to detect, treat, or prevent cancer.

It is contemplated that the inventive pharmaceutical compositions, TCRs, polypeptides, proteins, nucleic acids, recombinant expression vectors, host cells, or populations of cells can be used in methods of treating or preventing cancer. Without being bound to a particular theory, the inventive TCRs are believed to bind specifically to mutated target, e.g., mutated KRAS, such that the TCR (or related inventive polypeptide or protein), when expressed by a cell, is able to mediate an immune response against a target cell expressing mutated target, e.g., mutated KRAS. In this regard, the invention provides a method of treating or preventing cancer in a mammal, comprising administering to the mammal any of the pharmaceutical compositions. TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, in an amount effective to treat or prevent cancer in the mammal.

An embodiment of the invention provides any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, for use in the treatment or prevention of cancer in a mammal.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the cancer being treated or prevented. For example, treatment or prevention can include promoting the regression of a tumor. Also, for purposes herein, “prevention” can encompass delaying the onset of the cancer, or a symptom or condition thereof.

Also provided is a method of detecting the presence of cancer in a mammal. The method comprises (i) contacting a sample comprising one or more cells from the mammal with any of the inventive TCRs, polypeptides, proteins, nucleic acids, recombinant expression vectors, host cells, populations of cells, antibodies, or antigen binding portions thereof, or pharmaceutical compositions described herein, thereby forming a complex, and detecting the complex, wherein detection of the complex is indicative of the presence of cancer in the mammal.

With respect to the inventive method of detecting cancer in a mammal, the sample of cells can be a sample comprising whole cells, lysates thereof, or a fraction of the whole cell lysates, e.g., a nuclear or cytoplasmic fraction, a whole protein fraction, or a nucleic acid fraction.

For purposes of the inventive detecting method, the contacting can take place in vitro or in vivo with respect to the mammal. Preferably, the contacting is in vitro.

Also, detection of the complex can occur through any number of ways known in the art. For instance, the inventive TCRs, polypeptides, proteins, nucleic acids, recombinant expression vectors, host cells, populations of cells, or antibodies, or antigen binding portions thereof, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the inventive methods, wherein host cells or populations of cells are administered, the cells can be cells that are allogeneic or autologous to the mammal. Preferably, the cells are autologous to the mammal.

With respect to the inventive methods, the cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, colocrectal cancer, endometrial cancer, esophageal cancer, uterine cervical cancer, gastrointestinal carcinoid tumor, glioma. Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is cancer is pancreatic, colorectal, lung, endometrial, ovarian, or prostate cancer. Preferably, the lung cancer is lung adenocarcinoma, the ovarian cancer is epithelial ovarian cancer, and the pancreatic cancer is pancreatic carcinoma. In another preferred embodiment, the cancer is a cancer that expresses the mutated amino acid sequence of VVVGADGVGK (SEQ ID NO: 2), VVGADGVGK (SEQ ID NO: 34), VVVGAVGVGK (SEQ ID NO: 33), or VVGAVGVGK (SEQ ID NO: 35), which are present in mutated human KRAS, mutated human NRAS, and mutated human HRAS.

The mammal referred to in the inventive methods can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The following examples further illustrate the invention but, of course, should not be construed as in any way limiting its scope.

### Example 1

This example demonstrates the isolation of murine anti-KRAS7-16 G12D 10-mer TCRs.

A computer algorithm was used to generate candidate HLA-A11*01 KRAS peptides. For the algorithm, the strong binder threshold was 50 nM, and the weak binder threshold was 500 nM. The candidate peptides are shown in Table 1.

TABLE 1

| | SEQ ID | | HLA-A11*01 |
|---|---|---|---|
| Description | NO: | Sequence | (nM) |
| G12D 9-mer | 34 | VVGADGVGK | 194 |
| G12D 10-mer | 2 | VVVGADGVGK | 220 |
| G12V 9-mer | 35 | VVGAVGVGK | 50 |
| G12V 10-mer | 33 | VVVGAVGVGK | 71 |
| G12C 9-mer | 36 | VVGACGVGK | 69 |
| G12C 10-mer | 37 | VVVGACGVGK | 120 |
| G12R 9-mer | 38 | VVGARGVGK | 86 |
| G12R 10-mer | 39 | VVVGARGVGK | 119 |

HLA-A11 transgenic mice were immunized with the G12D 10-mer peptide (SEQ ID NO: 2) three times. After the third immunization, the spleen and lymph nodes were removed and cultured in vitro with the G12D 10-mer peptide at various concentrations (1 μM, 0.1 μM, and 0.01 μM) for seven days. T cells isolated from the lymph node (LN) and spleen cultures were tested for reactivity against (i) COS7 cells transduced to express HLA-A11 (COS7/A11) which had been pulsed with (a) no peptide (COS/A11), (b) WT KRAS7-16 peptide (SEQ ID NO: 30) (COS/A11+WT peptide), (c) G12D 10-mer peptide (SEQ ID NO: 2) (COS/A11+G12D peptide), or (d) G12V 10-mer peptide (SEQ ID NO: 33) (COS/A11+G12V peptide); and (ii) COS7/A11 cells transfected with a vector encoding a (a) WT KRAS minigene (encoding 23-mer SEQ ID NO: 118) (COS/A11/WT) or (b) G12D minigene (encoding 23-mer SEQ ID NO: 119) (COS/A11/G12D). Interferon (IFN)-γ was measured. The results are shown in Table 2A (pulsed target cells) and Table 2B (transfected target cells). As shown in Tables 2A and 2B, HLA-A11 restricted murine T cells were reactive against KRAS G12D peptide SEQ ID NO: 2.

TABLE 2A

| Stimu- | | mIFN-γ (pg/ml) | | | |
|---|---|---|---|---|---|
| lated | | | COS/A11+ | COS/A11+ | COS/A11+ |
| with G12D | | COS/ | WT | G12D | G12V |
| peptide | | A11 | peptide | peptide | peptide |
| 0.01 uM | LN-well | 50 | 34 | >20000 | 52 |
| | (W) 1 | | | | |
| | LN-W2 | 52 | 57 | >20000 | 95 |
| | LN-W3 | 169 | 92 | 12849 | 61 |
| | Spleen- | 32 | 32 | 45 | 33 |
| | W1 | | | | |
| | Spleen- | 35 | 50 | 57 | 44 |
| | W2 | | | | |
| | Spleen- | 68 | 72 | 94 | 40 |
| | W3 | | | | |
| 0.1 uM | LN-W1 | 38 | 38 | 16729 | 36 |
| | LN-W2 | 62 | 81 | >20000 | 81 |
| | LN-W3 | 73 | 116 | >20000 | 129 |
| | Spleen- | 36 | 43 | 14423 | 35 |
| | W1 | | | | |
| | Spleen- | 33 | 34 | >20000 | 33 |
| | W2 | | | | |
| | Spleen- | 44 | 40 | 18107 | 38 |
| | W3 | | | | |
| 1 uM | LN-W1 | 101 | 210 | >20000 | 407 |
| | LN-W2 | 92 | 248 | >20000 | 577 |
| | LN-W3 | 57 | 226 | >20000 | 403 |
| | Spleen- | 32 | 44 | >20000 | 55 |
| | W1 | | | | |
| | Spleen- | 34 | 70 | >20000 | 108 |
| | W2 | | | | |
| | Spleen- | 42 | 78 | >20000 | 261 |
| | W3 | | | | |

TABLE 2B

mIFN-γ (pg/ml)

| | COS/A11 | COS/A11/WT | COS/A11/G12D |
|---|---|---|---|
| Spleen-W1 | 32 | 32 | 19184 |
| Spleen-W2 | 34 | 36 | 19545 |
| Spleen-W3 | 42 | 45 | >20000 |
| LN-Wl | 101 | 74 | 6001 |
| LN-W2 | 92 | 147 | 13589 |
| LN-W3 | 57 | 64 | 11644 |
| Spleen-W1 | 36 | 53 | 12865 |
| Spleen-W2 | 33 | 49 | 12728 |
| Spleen-W3 | 44 | 45 | 12125 |
| LN-Wl | 38 | 44 | 7025 |
| LN-W2 | 62 | 54 | 19384 |
| LN-W3 | 73 | 66 | 17431 |
| Spleen-W1 | 32 | 32 | 52 |
| Spleen-W2 | 35 | 35 | 63 |
| Spleen-W3 | 68 | 36 | 94 |
| LN-Wl | 50 | 38 | 12096 |
| LN-W2 | 52 | 56 | 14098 |
| LN-W3 | 169 | 46 | 6877 |

The TCR was isolated from the cells in each positive well using 5′ Rapid Amplification of cDNA Ends (RACE). Two dominant alpha chains and four dominant beta chains were identified (Table 3).

TABLE 3

| | V Region | D/J Region | CDR3 | SEQ ID NO: |
|---|---|---|---|---|
| Alpha | TRAV12N-3*01 | 39*01 | CALRGNAGAKLTF | 5 |
| chains | TRAV16D/DV11*03 | 52*01 | CAMREDTGANTGKLTF | 40 |
| Beta | TRBV4*01 (CB2) | 2*01/2-3*01 | CASSSRDWSAETLYF | 8 |
| chains | TRBV5*01 (CB2) | 2*01/2-1*01 | CASSQDSLGRAEQFF | 41 |
| | TRBV16*01 (CB2) (LN 0.01) | 2*01/2-3*01 | CASSSDWGGAETLYF | 42 |
| | TRBV16*01 (CB2) (Spl) | 2*01/2-3*01 | CASSSGLGSSAETLYF | 43 |

### Example 2

This example demonstrates that PBL transduced to express a TCR α chain comprising SEQ ID NO: 11 and a TCR β chain comprising SEQ ID NO: 12 are reactive against HLA-A11+/G12D 10-mer+ targets.

The two dominant α chains and four dominant β chains of Table 3 were individually cloned into MSGV1 retroviral vectors. PBL were individually co-transduced to express one of various pairs of an α and β chain, as shown in Table 4. Transduced PBL were screened for reactivity against (i) HLA-A11-expressing T2/A11+ (Table 4A) or COS7/A11+ (Table 4B) cells pulsed with (a) G12D 10-mer peptide (SEQ ID NO: 2), (b) G12V 10-mer peptide (SEQ ID NO: 33), (c) WT KRAS 10-mer peptide (SEQ ID NO: 30), or (d) no peptide (none); or (ii) COS7/A11 cells transduced with a (a) G12D minigene (encoding 23-mer SEQ ID NO: 119) (COS/A11/G12D), (b) G12V minigene (encoding 23-mer SEQ ID NO: 120) (COS/A11/G12V), (c) WT KRAS minigene (encoding 23-mer SEQ ID NO: 118) (COS/A11/WT), or (d) no cells (medium only) (Table 4C). IFN-γ secretion was measured. The results are shown in Tables 4A-C. In Tables 4A-C, bold IFN-γ secretion values indicate those pairs of TCR α and β chains that demonstrated reactivity, and IFN-γ secretion values in bold with underlining indicate the pair of TCR α and β chains that demonstrated the best reactivity. As shown in Tables 4A-C, PBL co-transduced to express murine TCR α chain TRAV12N-3*01 (SEQ ID NO: 11) and murine TCR β chain TRBV4*01 (SEQ ID NO: 12) demonstrated reactivity against HLA-A11-expressing COS7 cells pulsed with G12D 10-mer or G12D transfectant target cells.

TABLE 4A

| | | IFN-γ (pg/ml) upon co-culture | | | |
|---|---|---|---|---|---|
| | | with T2/A11+ target cells | | | |
| | | | WT 10- | G12D 10- | G12V |
| | | None | mer | mer | 10-mer |
| TRAV12N- | TRBV4*01 | 108 | 136 | >10000 | 108 |
| 3*01 | TRBV5*01 | 348 | 138 | 319 | 263 |
| | TRBV16*01 | 107 | 100 | 93 | 120 |
| | (LN) | | | | |
| | TRBV16*01 | 234 | 132 | 246 | 132 |
| | (SP) | | | | |
| TRAV16D/ | TRBV4*01 | 56 | 39 | 595 | 39 |
| DV11*03 | TRBV5*01 | 140 | 146 | 848 | 155 |
| | TRBV16*01 | 71 | 100 | 135 | 51 |
| | (LN) | | | | |
| | TRBV16*01 | 228 | 297 | 133 | 144 |
| | (SP) | | | | |

TABLE 4B

| | | IFN-γ (pg/ml) upon co- | | | |
|---|---|---|---|---|---|
| | | culture with COS/A11+ targets | | | |
| | | | WT r10- | G12D | G12V |
| | | None | mer | 10-mer | 10-mer |
| TRAV12N- | TRBV4*01 | 123 | 107 | >10000 | 129 |
| 3*01 | TRBV5*01 | 57 | 71 | 86 | 58 |
| | TRBV16*01 (LN) | 55 | 69 | 70 | 81 |
| | TRBV16*01 (Sp) | 98 | 64 | 71 | 78 |
| TRAV16D/ | TRBV4*01 | 71 | 57 | 246 | 71 |
| DV11*03 | TRBV5*01 | 74 | 66 | 1228 | 70 |
| | TRBV16*01 (LN) | 74 | 77 | 68 | 85 |
| | TRBV16*01 (Sp) | 108 | 121 | 104 | 100 |

TABLE 4C

| | | IFN-γ (pg/ml) upon co- | | | |
|---|---|---|---|---|---|
| | | culture with target cells | | | |
| | | COS/ | COS/ | COS/ | |
| | | A11/WT | A11/G12D | A11/G12V | Medium |
| TRAV12N- | TRBV4*01 | 130 | >10000 | 126 | 18 |
| 3*01 | TRBV5*01 | 95 | 106 | 83 | 22 |
| | TRBV16*01 | 97 | 91 | 98 | 18 |
| | (LN) | | | | |
| | TRBV16*01 | 129 | 114 | 84 | 23 |
| | (SP) | | | | |
| TRAV16D/ | TRBV4*01 | 95 | 302 | 89 | 18 |
| DV11*03 | TRBV5*01 | 94 | 92 | 98 | 18 |
| | TRBV16*01 | 99 | 106 | 114 | 24 |
| | (LN) | | | | |
| | TRBV16*01 | 176 | 143 | 138 | 26 |
| | (SP) | | | | |

### Example 3

This example demonstrates that PBL co-transduced with a TCR α chain comprising SEQ ID NO: 11 and a TCR β chain comprising SEQ ID NO: 12 are reactive against HLA-A11+/G12D+ pancreatic tumor cell line FA6-2/A11.

Human PBL were co-transduced with a TCR α chain comprising SEQ ID NO: 11 and a TCR β chain comprising SEQ ID NO: 12. Co-transduced cells were co-cultured with (i) COS7 cells transfected with (a) HLA-A11 alone (COS7/A11) or HLA-A11 transduced with a (b) WT KRAS minigene (encoding 23-mer SEQ ID NO: 118) (COS7/A11/KRAS WT), (c) KRAS G12D minigene (encoding 23-mer SEQ ID NO: 119) (COS7/A11/KRAS G12D), (d) KRAS G12V minigene (encoding 23-mer SEQ ID NO: 120) (COS7/A11/KRAS G12V); (ii) pancreatic tumor cell lines Mia-Paca2/A11, T3m4/A11, AsPC-1, FA6-2/A11, MDA-Panc-48/A11, PANC-1, PK-45p/A11, SK.PC.3/A11, x135 ml/A11, or (iii) medium alone. IFN-γ secretion was measured. The results are shown in Table 5. The KRAS mutations of the tumor cell lines are indicated in parentheses. As shown in Table 5, PBL co-transduced with a TCR α chain TRAV12N-3*01 (SEQ ID NO: 11) and TCR β chain TRBV4*01 (SEQ ID NO: 12) demonstrated reactivity against HLA-A11+/G12D+ pancreatic tumor cell line FA6-2/A11.

TABLE 5

| Target Cell | IFN-γ (pg/mL) |
|---|---|
| COS7/A11 | 146 |
| COS7/A11/KRAS WT | 116 |
| COS7/A11/KRAS (G12D) | 18231 |
| COS7/A11/KRAS (G12V) | 111 |
| Mia-Paca2/A11 (G12C)* | 53 |
| T3m4/A11 (Q61H)* | 178 |
| SK.PC.3/A11 (G12V)** | 53 |
| x135m1/A11 (G12V)** | 105 |
| AsPC-1 (G12D)** | 18 |
| FA6-2/A11 (G12D)** | 3982 |
| MDA-Panc-48/A11 (G12D)** | 56 |
| PANC-1 (A11+, G12D)** | 28 |
| PK.45p/A11 (G12D)** | 231 |
| Medium (no cells) | 26 |

*Mutation determined by genotyping.
**Mutation determined by enotypin and mRNA expression (see Tables 13 and 20).

### Example 4

This example demonstrates that PBL that were transduced with a retroviral vector encoding a TCR α chain TRAV12N-3*01 (SEQ ID NO: 11) and TCR β chain TRBV4*01 (SEQ ID NO: 12) demonstrated reactivity against COS7/A11 cells pulsed with KRAS G12D 10-mer peptide (SEQ ID NO: 2).

Human PBL were transduced with a retroviral vector encoding the TCR α chain TRAV12N-3*01 (SEQ ID NO: 11) and TCR β chain TRBV4*01 (SEQ ID NO: 12). Transduced PBL were co-cultured with COS7/All cells that were pulsed with KRAS G12D 10-mer peptide (SEQ ID NO: 2), KRAS G12D 9-mer peptide (SEQ ID NO: 34), KRAS G12D 9-mer peptide SEQ ID NO: 124, KRAS G12V 10-mer peptide (SEQ ID NO: 33), or WT KRAS 10-mer peptide (SEQ ID NO: 30) at various concentrations shown in Table 6. IFN-γ secretion was measured. The results are shown in Table 6. As shown in Table 6, human PBL transduced to express a TCR α chain TRAV12N-3*01 (SEQ ID NO: 11) and TCR β chain TRBV4*01 (SEQ ID NO: 12) demonstrated reactivity against COS7/A11 cells pulsed with KRAS G12D 10-mer peptide (SEQ ID NO: 2).

TABLE 6

| | IFN-γ (pg/mL) | | | | |
|---|---|---|---|---|---|
| Peptide | | G12D KRAS₇₋₁₅ | | G12D KRAS₈₋₁₆ | |
| concen- | G12D | 9-mer | G12V | 9-mer | WT |
| tration | 10- | (VVVGADGVG) | 10- | (VVGADGVGK) | 10- |
| (M) | mer | SEQ ID NO: 124 | mer | SEQ ID NO: 34 | mer |
| 1 × 10⁻⁶ | 16918 | 87 | 97 | 136 | 78 |
| 1 × 10⁻⁷ | 8677 | 91 | 83 | 95 | 88 |
| 1 × 10⁻⁸ | 4220 | 72 | 86 | 99 | 102 |
| 1 × 10⁻⁹ | 775 | 90 | 88 | 90 | 99 |
| 1 × 10⁻¹⁰ | 115 | 90 | 88 | 85 | 95 |
| 1 × 10⁻¹¹ | 98 | 95 | 86 | 85 | 86 |
| 1 × 10⁻¹² | 111 | 83 | 96 | 94 | 102 |
| 1 × 10⁻¹³ | 80 | 112 | 97 | 115 | 98 |

### Example 5

This example demonstrates that PBL that were transduced with a retroviral vector encoding a TCR α chain TRAV12N-3*01 (SEQ ID NO: 11) and TCR β chain TRBV4*01 (SEQ ID NO: 12) demonstrated reactivity against HLA-A11-expressing pancreatic tumor line FA6-2/A11.

Human PBL were transduced with a retroviral vector encoding the TCR α chain TRAV12N-3*01 (SEQ ID NO: 11) and TCR β chain TRBV4*01 (SEQ ID NO: 12). Untransduced control PBL or transduced PBL were co-cultured with the target cells set forth in Table 7. IFN-γ secretion was measured. The results are shown in Table 7. The KRAS mutations of the tumor cell lines are indicated in parentheses. As shown in Table 7, human PBL transduced to express a TCR α chain TRAV12N-3*01 (SEQ ID NO: 11) and TCR β chain TRBV4*01 (SEQ ID NO: 12) demonstrated reactivity against FA6-2/A11 tumor cell line. Untransduced PBL secreted less than 100 pg/mL IFN-γ upon co-culture with each target cell set forth in Table 7.

TABLE 7

| Target Cell | IFN-γ (pg/mL) |
|---|---|
| COS7/A11 | 90 |
| COS7/A11/KRAS WT | 71 |
| COS7/A11/KRAS (G12D) | 15496 |
| COS7/A11/KRAS (G12V) | 58 |
| Barr (A11+, G12R)* | 21 |
| BxPC3/A11 (WT)* | 18 |
| Mia-Paca2/A11 (G12C)* | 57 |
| Paca44/A11 (G12V)** | 30 |
| T3m4/A11 (Q61H)* | 28 |
| AsPC-1/A11 (G12D)** | 60 |
| FA6-2/A11 (G12D)** | 753 |
| MDA-Panc-48/A11 (G12D)** | 23 |
| PANC-1 (A11+, G12D)** | 23 |
| PK.45p/A11 (G12D)** | 28 |
| Medium (no cells) | 38 |

*Mutation (or lack thereof (i.e., “WT”) determined by genotyping.
**Mutation determined by genotyping and mRNA expression (see Tables 13 and 20).

### Example 6

This example demonstrates the isolation of murine anti-KRAS7-16 G12V 10-mer TCRs.

HLA-A11 transgenic mice were immunized with the G12V 10-mer peptide (SEQ ID NO: 33) twice. After the second immunization, the spleen and lymph nodes were removed and cultured in vitro with the G12V 10-mer peptide at various concentrations (1 μM, 0.1 μM, and 0.01 μM) for seven days. T cells isolated from the lymph node and spleen cultures were tested for reactivity against (i) COS7/A11 cells transfected with a vector encoding (a) G12V minigene (encoding 23-mer SEQ ID NO: 120) (COSA11/G12V); (b) WT KRAS minigene (encoding 23-mer SEQ ID NO: 118) (COSA11/WT); (c) G12D minigene (encoding 23-mer SEQ ID NO: 119) (COSA11/G12D); (ii) HLA-A11-expressing KRAS G12V+ pancreatic tumor cell lines Paca44/A11, SKPC3/A11, or x135 ml/A11; or (iii) no target cells (medium) (Table 8). The results are shown in Table 8. In Table 8, underlined IFN-γ secretion values indicate those cells that demonstrated reactivity against transfectants and tumors.

TABLE 8

| | mIFN-γ (pg/ml) | | | | | |
|---|---|---|---|---|---|---|
| | | | Spleen | | LN | LN |
| | Spleen | Spleen | (0.01 | LN | (0.1 | (0.01 |
| | (1 μM) | (0.1 μM) | μM) | (1 μM) | μM) | μM) |
| Cos7/ | 34 | 32 | 32 | 38 | 46 | 37 |
| A11/WT | | | | | | |
| Cos7/A11/ | 61 | 32 | 32 | 41 | 41 | 38 |
| G12D | | | | | | |
| Cos7/A11/ | >20000 | 12113 | 58 | 1685 | 3126 | 4765 |
| G12V | | | | | | |
| Paca44/A11 | 32 | 32 | 36 | 39 | 44 | 39 |
| SKPC3/A11 | 8235 | 385 | 41 | 106 | 169 | 164 |
| x135ml/A11 | 32 | 36 | 49 | 42 | 40 | 41 |
| Medium | 32 | 32 | 50 | 46 | 35 | 42 |

Oligoclonal TCRs were isolated from the cells that demonstrated highly specific G12V peptide and transfectant reactivity using 5′ RACE. Two dominant alpha chains and three dominant beta chains were identified (Table 9).

TABLE 9

| | V Region | D/J Region | CDR3 | SEQ ID NO: | Frequency |
|---|---|---|---|---|---|
| Alpha | TRAV19*01 | 53*01 | CAAGDSGGSNYKLTF | 139 | 31% |
| chains | TRAV3-3*01 | 17*01 | CAVSGGTNSAGNKLTF | 204 | 14% |
| Beta | TRBV13-1*02 (CB2) | 2*01/2-1*01 | CASASWGGYAEQFF | 205 | 23% |
| chains | TRBV4*01 (CB2) | 2*01/2-1*01 | CASSRDWGPAEQFF | 130 | 15% |
| | TRBV1*01 (CB2) | 1*01/2-3*01 | CTCSADRGAETLYF | 206 | 12% |

### Example 7

This example demonstrates that PBL transduced to express (i) a TCR α chain comprising SEQ ID NO: 133 and a TCR β chain comprising SEQ ID NO: 134 or (ii) a TCR α chain comprising SEQ ID NO: 145 and a TCR β chain comprising SEQ ID NO: 146 are reactive against HLA-A11+/G12V 10-mer+ targets.

The two dominant α chains and three dominant β chains of Table 9 were individually cloned into MSGV1 retroviral vectors. Anti-CD3 stimulated PBL were individually co-transduced to express one of various pairs of an α and β chain, as shown in Tables 10A-10B. Transduced PBL were screened for reactivity against (i) COS7/A11+ cells pulsed with (a) G12D 10-mer peptide (SEQ ID NO: 2), (b) G12V 10-mer peptide (SEQ ID NO: 33), or (c) WT KRAS 10-mer peptide (SEQ ID NO: 30) (Table 10A) or (ii) COS7/A11 cells transduced with a (a) G12D minigene (encoding 23-mer SEQ ID NO: 119) (COS/A11/G12D), (b) G12V minigene (encoding 23-mer SEQ ID NO: 120) (COS/A11/G12V), or (c) WT KRAS minigene (encoding 23-mer SEQ ID NO: 118) (COS/A11/WT) (Table 10B). Untransfected Cos7/A11 cells that were not pulsed with peptide (Cos7/A11) and medium with no cells (medium) served as negative controls. PBL pulsed or transduced with GFP served as a positive control. IFN-γ secretion was measured.

The results are shown in Tables 10A-10B. In Tables 10A-10B, bold IFN-γ secretion values indicate those pairs of TCR α and β chains that demonstrated reactivity. As shown in Tables 10A-10B, PBL co-transduced to express (i) both murine TCR α chain TRAV19*01 (SEQ ID NO: 145) and murine TCR β chain TRBV13-1*02 (SEQ ID NO: 146) or (ii) both murine TCR α chain TRAV3-3*01 (SEQ ID NO: 133) and murine TCR β chain TRBV4*01 (SEQ ID NO: 134) demonstrated reactivity against HLA-A11-expressing COS7 cells pulsed with G12V 10-mer or G12V transfectant target cells, but not control peptides or control transfectants.

TABLE 10A

| | (IFN-γ (pg/ml)) | | | | |
|---|---|---|---|---|---|
| | Cos7/ | Cos7/A11 + | Cos7/A11 + | Cos7/A11 + | Me- |
| | A11 | WT 10-mer | G12D 10-mer | G12V 10-mer | dium |
| GFP | 66 | 50 | 60 | 54 | 17 |
| TRAV19*01 + | 105 | 90 | 94 | 14138 | 29 |
| TRBV13-1*02 | | | | | |
| TRAV19*01 + | 30 | 30 | 30 | 27 | 16 |
| TRBV4*01 | | | | | |
| TRAV19*01 + | 69 | 37 | 38 | 37 | 16 |
| TRBV1*01 | | | | | |
| TRAV3-3*01 + | 68 | 47 | 49 | 44 | 23 |
| TRBV13-1*02 | | | | | |
| TRAV3-3*01 + | 42 | 36 | 39 | 8374 | 16 |
| TRBV4*01 | | | | | |
| TRAV3-3*01 + | 53 | 41 | 39 | 51 | 16 |
| TRBV1*01 | | | | | |

TABLE 10B

| | (IFN-γ (pg/ml)) | | | | |
|---|---|---|---|---|---|
| | Cos7/ | Cos7/A11/ | Cos7/A11/ | Cos7/A11/ | Me- |
| | A11 | WT | G12D | G12V | dium |
| GFP | 66 | 72 | 60 | 55 | 17 |
| TRAV19*01 + | 105 | 92 | 81 | 18058 | 29 |
| TRBV13-1*02 | | | | | |
| TRAV19*01 + | 30 | 32 | 27 | 30 | 16 |
| TRBV4*01 | | | | | |
| TRAV19*01 + | 69 | 45 | 45 | 44 | 16 |
| TRBV1*01 | | | | | |
| TRAV3-3*01 + | 68 | 51 | 56 | 61 | 23 |
| TRBV13-1*02 | | | | | |
| TRAV3-3*01 + | 42 | 41 | 38 | 11113 | 16 |
| TRBV4*01 | | | | | |
| TRAV3-3*01 + | 53 | 44 | 47 | 45 | 16 |
| TRBV1*01 | | | | | |

### Example 8

This example demonstrates that the TRAV3-3*01/TRBV4*01 murine anti-KRAS G12V TCR (SEQ ID NOs: 133 and 134) has a higher affinity for pulsed target peptide as compared to TRAV19*01/TRBV13-1*02 murine anti-KRAS G12V TCR (SEQ ID NOs: 145 and 146).

PBL were transduced with either (i) TRAV3-3*01/TRBV4*01 murine anti-KRAS G12V TCR (SEQ ID NOs: 133 and 134) or (ii) TRAV19*01/TRBV13-1*02 murine anti-KRAS G12V TCR (SEQ ID NOs: 145 and 146). Transduced cells were co-cultured with Cos7/A11 cells pulsed with (a) G12D 10-mer peptide (SEQ ID NO: 2), (b) G12V 10-mer peptide (SEQ ID NO: 33), (c) WT KRAS 10-mer peptide (SEQ ID NO: 30), (d) G12D 9-mer peptide (SEQ ID NO: 34), or (e) G12V 9-mer peptide (SEQ ID NO: 35) at the concentrations shown in Tables 11A and 11B. IFN-γ secretion was measured.

The results are shown in Table 11A (TRAV3-3*01/TRBV4*01 (SEQ ID NOS: 133 and 134)) and Table 11B (TRAV19*01/TRBV13-1*02 (SEQ ID NOs: 145 and 146)). In Tables 11A-11B, bold IFN-γ secretion values indicate those target peptide concentrations at which the TCR demonstrated reactivity. As shown in Tables 11A-11B, T cells transduced with the TRAV3-3*01/TRBV4*01 TCR (SEQ ID NOs: 133 and 134) recognized Cos7/All pulsed with both 9-mer and 10-mer peptides and recognized 9-mer at pulsed at a concentration of 0.01 nM. Accordingly, the TRAV3-3*01/TRBV4*01 TCR (SEQ ID NOS: 133 and 134) recognized pulsed target peptide with a higher avidity as compared to the TRAV19*01/TRBV13-1*02 (SEQ ID NOs: 145 and 146) TCR. The increased reactivity of the TRAV3-3*01/TRBV4*01 (SEQ ID NOs: 145 and 146) TCR against the G12V 9-mer peptide as compared to the 10-mer peptide also suggested that 9-mer peptide is the minimal determinant.

TABLE 11A

| Peptide | IFN-γ (pg/ml) | | | | |
|---|---|---|---|---|---|
| concentration | WT | G12D | G12D | G12V | G12V |
| (10^{×} μM) | 10-mer | 9-mer | 10-mer | 9-mer | 10-mer |
| −6 | 50 | 47 | 43 | 19479 | 9778 |
| −7 | 42 | 48 | 40 | 19900 | 6696 |
| −8 | 50 | 49 | 46 | 19193 | 657 |
| −9 | 50 | 44 | 41 | 9578 | 104 |
| −10 | 48 | 52 | 53 | 1877 | 59 |
| −11 | 55 | 49 | 43 | 119 | 52 |
| −12 | 47 | 55 | 49 | 56 | 52 |
| −13 | 68 | 52 | 49 | 60 | 52 |

TABLE 11B

| Peptide | IFN-γ (pg/ml) | | | | |
|---|---|---|---|---|---|
| concentration | WT | G12D | G12D | G12V | G12V |
| (10^{×} μM) | 10-mer | 9-mer | 10-mer | 9-mer | 10-mer |
| −6 | 57 | 63 | 61 | 112 | 15184 |
| −7 | 56 | 57 | 50 | 70 | 7725 |
| −8 | 57 | 48 | 49 | 52 | 2084 |
| −9 | 49 | 54 | 59 | 55 | 326 |
| −10 | 57 | 62 | 52 | 64 | 61 |
| −11 | 65 | 52 | 64 | 62 | 67 |
| −12 | 67 | 57 | 62 | 66 | 61 |
| −13 | 70 | 70 | 63 | 64 | 71 |

### Example 9

This example demonstrates that the TRAV3-3*01/TRBV4*01 murine anti-KRAS G12V TCR (SEQ ID NOs: 133 and 134) recognizes HLA-A11+KRAS G12V+ pancreatic tumor cell lines.

PBL were transduced with either (i) TRAV3-3*01/TRBV4*01 murine anti-KRAS G12V TCR (SEQ ID NOs: 133 and 134) or (ii) TRAV19*01/TRBV13-1*02 murine anti-KRAS G12V TCR (SEQ ID NOs: 145 and 146). Transduced cells were co-cultured with (i) COS7/A11 cells transduced with a (a) G12D minigene (encoding 23-mer SEQ ID NO: 119) (COS/A11/G12D), (b) G12V minigene (encoding 23-mer SEQ ID NO: 120) (COS/A11/G12V), or (c) WT KRAS minigene (encoding 23-mer SEQ ID NO: 118) (COS/A11/WT); (ii) KRAS G12V negative pancreatic tumor cell lines transduced with HLA-A11; (iii) KRAS G12V+ pancreatic tumor cell lines transduced with HLA-A11; or (iv) parental (untransduced) pancreatic tumor cell lines, as shown in Table 12. IFN-γ secretion was measured.

The results are shown in Table 12. In Table 12, bold IFN-γ secretion values indicate those target cells for which the TCR demonstrated reactivity. As shown in Table 12, the TRAV3-3*01/TRBV4*01 murine anti-KRAS G12V TCR (SEQ ID NOs: 133 and 134) recognized more HLA-A11+KRAS G12V+ pancreatic tumor cell lines as compared to the TRAV19*01/TRBV13-1*02 murine anti-KRAS G12V TCR (SEQ ID NOs: 145 and 146).

TABLE 12

| | | IFN-γ (pg/ml) | |
|---|---|---|---|
| | | TRAV3-3*01/ | TRAV19*01/ |
| | | TRBV4*01 | TRBV13-1*02 |
| Transfectants | Cos/A11 | 51 | 55 |
| | Cos/A11/G12D | 53 | 44 |
| | Cos/All/WT | 52 | 51 |
| | Cos/A11/G12V | 24290 | 11794 |
| HLA-A11 | BxPC3/A11 (WT)* | 25 | 32 |
| transduced, | MiaPaca2/A11 (G12C)* | 16 | 16 |
| KRAS G12V− | T3m4/A11 (Q61H)* | 26 | 33 |
| | AsPC-1/A11 (G12D)** | 22 | 26 |
| | FA6-2/A11 (G12D)** | 18 | 18 |
| | MDA-Panc-48/A11 | 16 | 16 |
| | (G12D)** | | |
| | PK.45p/A11 (G12D)** | 31 | 29 |
| HLA-A11 | Capan-1/A11 (G12V)** | 99 | 28 |
| transduced, | CFPAC-1/A11 (G12V)** | 224 | 28 |
| KRAS | Paca44/A11 (G12V)** | 577 | 21 |
| G12V+ | SK.PC3/A11 (G12V)** | 7947 | 2658 |
| | x135m1/A11 (G12V)** | 1020 | 90 |
| Parental | BxPC3 (WT)* | 16 | 16 |
| tumor lines | MiaPaca2 (G12C)* | 18 | 16 |
| | T3m4 (Q61H)* | 19 | 18 |
| | AsPC-1 (G12D)** | 16 | 16 |
| | FA6-2 (G12D)** | 23 | 19 |
| | MDA-Panc-48 (G12D)** | 19 | 21 |
| | PK.45p (G12D)** | 16 | 16 |
| | Capan-1 (G12V)** | 22 | 20 |
| | CFPAC-1 (G12V)** | 16 | 16 |
| | Paca44 (G12V)** | 16 | 16 |
| | SK.PC3 (G12V)** | 28 | 19 |
| | x135m1 (G12V)** | 27 | 20 |
| HLA-A11+ | PANC-1 (HLA-A11+, | 16 | 17 |
| G12V− | G12D)** | | |
| | Barr (HLA-A11+, G12R)* | 17 | 22 |
| | Medium | 18 | 16 |

*Mutation (or lack thereof, i.e., “WT”) determined by genotyping.
**Mutation determined by genotyping and mRNA expression (see Tables 13 and 20).

### Example 10

This example demonstrates the correlation between IFN-γ production and mutated KRAS expression for the TRAV3-3*01/BV4*01 murine anti-KRAS G12V TCR (SEQ ID NOs: 133 and 134).

The number of copies of KRAS G12V mRNA expressed by each of the pancreatic tumor cell lines shown in Table 13 was measured and compared to the number of copies of β-actin mRNA expressed by the indicated cell line (Table 13). The amount of IFN-γ secreted by the PBL transduced with the TRAV3-3*01/BV4*01 murine anti-KRAS G12V TCR (SEQ ID NOs: 133 and 134) upon co-culture with each cell line measured in Example 9 is reproduced in Table 13. The reactivity of the TRAV3-3*01/BV4*01 murine anti-KRAS G12V TCR (SEQ ID NOs: 133 and 134) (in terms of IFN-γ secretion upon co-culture with target cells) correlated with the number of copies of KRAS G12V mRNA.

TABLE 13

| | β-actin | Ref Total KRAS | G12V (Copy | |
|---|---|---|---|---|
| | (Copy | (Copy per 10⁶ | per 10⁶ | IFN-γ |
| | number) | β-actin) | β-actin) | (pg/ml) |
| BxPC3/A11 | 3.13 × 10⁷ | 6.84 × 10³ | 2.51 | 25 |
| MiaPaca2/A11 | 2.01 × 10⁷ | 5.87 × 10³ | 1.06 × 10⁻¹ | 16 |
| Capan-1/A11 | 2.28 × 10⁷ | 5.92 × 10³ | 5.42 × 10³ | 99 |
| CFPAC-1/A11 | 1.96 × 10⁷ | 2.09 × 10⁴ | 3.72 × 10³ | 224 |
| Paca44/A11 | 1.80 × 10⁷ | 4.94 × 10³ | 3.62 × 10³ | 577 |
| SK.PC3/A11 | 3.28 × 10⁷ | 1.48 × 10⁴ | 1.42 × 10⁴ | 7947 |
| x135m1/A11 | 8.50 × 10⁶ | 8.75 × 10³ | 9.85 × 10³ | 1020 |

### Example 11

This example demonstrates that the TRAV3-3*01/BV4*01 murine anti-KRAS G12V TCR (SEQ ID NOs: 133 and 134) recognizes mutated KRAS either (i) in the presence of CD4 and the absence of CD8 or (ii) in the presence of CD8 and the absence of CD4.

PBL were transduced with a nucleotide sequence encoding the TRAV3-3*01/BV4*01 murine anti-KRAS G12V TCR (SEQ ID NOs: 133 and 134). Transduced cells were sorted into the populations shown in Table 14 by flow cytometry. The sorted populations of cells were co-cultured with (i) COS7/All cells transduced with a (a) G12D minigene (encoding 163-mer) (COS/A11/G12D), (b) G12V minigene (encoding 163-mer) (COS/A11/G12V), or (c) WT KRAS minigene (encoding 163-mer) (COS/A11/WT); or (ii) SK.PC3 pancreatic tumor cell line untransduced or transduced with HLA-A11. Medium without cells served as a negative control. IFN-γ secretion was measured.

The results are shown in Table 14. In Table 14, bold IFN-γ secretion values indicate those target cells for which the TCR demonstrated reactivity. As shown in Table 14, the TRAV3-3*01/BV4*01 murine anti-KRAS G12V TCR (SEQ ID NOs: 133 and 134) recognized target cells either (i) in the presence of CD4 and the absence of CD8 or (ii) in the presence of CD8 and the absence of CD4. Accordingly, the TRAV3-3*01/BV4*01 murine anti-KRAS G12V TCR (SEQ ID NOs: 133 and 134) provides highly avid recognition of the target.

TABLE 14

| PBL | | | | | | |
|---|---|---|---|---|---|---|
| transduced | | | | | | |
| with | IFN-g (pg/ml) | | | | | |
| TRAV3-3*01/BV4*01 | Cos7/A11/WT | Cos7/A11/G12D | Cos7/A11/G12V | SK.PC3 | SK.PC3/A11 | Medium |
| Bulk | 48 | 67 | 8294 | 41 | 8944 | 16 |
| CD8 | 49 | 64 | 8150 | 75 | 8602 | 16 |
| enriched | | | | | | |
| CD4 | 16 | 16 | 763 | 16 | 458 | 16 |
| enriched | | | | | | |
| GFP | 16 | 17 | 16 | 20 | 16 | 16 |

### Example 12

This example demonstrates the isolation of murine anti-KRAS7-16 G12D 10-mer TCRs.

HLA-A11 transgenic mice were immunized with the G12D 10-mer peptide (SEQ ID NO: 2) three times. After the third immunization, the spleen and lymph nodes were removed and cultured in vitro with the G12D 10-mer peptide at various concentrations (1 μM, 0.1 μM, and 0.01 μM) for seven days. T cells isolated from the LN and spleen cultures were tested for reactivity against (i) COS7 cells transduced to express HLA-A11 (COS7/A11) which had been pulsed with (a) no peptide (none), (b) WT KRAS7-16 peptide (SEQ ID NO: 30) (COS/A11+WT peptide), (c) G12D 10-mer peptide (SEQ ID NO: 2) (COS/A11+G12D peptide), (d) G12V 10-mer peptide (SEQ ID NO: 33) (COS/A11+G12V peptide), (e) G12V 9-mer peptide (SEQ ID NO: 35), or (f) G12D 9-mer peptide (SEQ ID NO: 34); and (ii) COS7/A11 cells transfected with a vector encoding a (a) WT KRAS minigene (encoding 23-mer SEQ ID NO: 118) (COS/A11/WT), (b) G12D minigene (encoding 23-mer SEQ ID NO: 119) (COS/A11/G12D), or (c) (b) G12V minigene (encoding 23-mer SEQ ID NO: 120) (COS/A11/G12D). Interferon (IFN)-γ was measured.

The results are shown in Table 15A (peptide pulse) and Table 15B (transfectants). In Tables 15A and 15B, bold IFN-γ secretion values indicate those target peptides and target cells for which the TCR demonstrated reactivity. As shown in Tables 15A and 15B, HLA-A11 restricted murine T cells were reactive against KRAS G12D peptide SEQ ID NO: 2.

TABLE 15A

| | Stimulated | Cos7/A11 pulsed (IFN-y (pg/ml)) | | | | | |
|---|---|---|---|---|---|---|---|
| | with | | WT | G12D | G12D | G12V | G12V |
| | G12D10-mer | none | 10-mer | 9-mer | 10-mer | 9-mer | 10-mer |
| Spleen | 1 μM | 55 | 49 | 121 | >20000 | 54 | 64 |
| | 0.1 μM | 65 | 59 | 120 | 19521 | 65 | 64 |
| | 0.01 μM | 82 | 61 | 87 | 1060 | 66 | 66 |
| LN | 1 μM | 57 | 98 | 807 | >20000 | 68 | 95 |
| | 0.1 μM | 80 | 86 | 375 | >20000 | 108 | 89 |
| | 0.01 μM | 349 | 339 | 435 | >20000 | 296 | 325 |

TABLE 15B

| | (IFN-γ (pg/ml)) | | |
|---|---|---|---|
| | Cos7/A11/WT | Cos7/A11/G12D | Cos7/A11/G12V |
| Spleen | 50 | >20000 | 49 |
| | 53 | >20000 | 59 |
| | 80 | 847 | 81 |
| LN | 71 | >20000 | 75 |
| | 235 | >20000 | 102 |
| | 440 | >20000 | 328 |

T cells isolated from the LN and spleen cultures were also stimulated in vitro with various concentrations of G12D peptides for 6-7 days and were then co-cultured with the HLA-A11-expressing, KRAS G12D+ pancreatic cell lines shown in Table 16. IFN-γ was measured.

The results are shown in Table 16. In Table 16, bold IFN-γ secretion values indicate those target cells for which the TCR demonstrated reactivity. As shown in Table 16, T cells isolated from the LN and spleen cultures were reactive with HLA-A11-expressing, KRAS G12D+ pancreatic cell lines.

TABLE 16

| | stimulated | IFN-y (pg/ml) | | | | |
|---|---|---|---|---|---|---|
| | with G12D10-mer | Medium | FA6-2/A11 | MDA-Panc48/A11 | Panc-1 | PK.45p/A11 |
| Spleen | 1 μM | 47 | 2134 | 1322 | 48 | 46 |
| | 0.1 μM | 46 | 1588 | 665 | 54 | 54 |
| | 0.01 μM | 59 | 116 | 443 | 54 | 59 |
| LN | 1 μM | 55 | 4614 | 202 | 61 | 57 |
| | 0.1 μM | 121 | 4512 | 211 | 68 | 74 |
| | 0.01 μM | 279 | 3019 | 559 | 96 | 249 |

The TCR was isolated from the reactive cells using 5′ RACE. Two dominant alpha chains and one dominant beta chain were identified (Table 17).

TABLE 17

| | V Region | D/J Region | CDR3 | SEQ ID NO: | Frequency |
|---|---|---|---|---|---|
| Alpha | TRAV4- | 49*01 | CAADSSNTGYQNFYF | 151 | 30% |
| chains | 4/DV10*01(1) | | | | |
| | TRAV4- | 49*01 | CAALNTGYQNFYF | 161 | 10% |
| | 4/DV10*01(2) | | | | |
| Beta | TRBV12-2*01 | 1*01/1- | CASSLTDPLDSDYTF | 154 | 18% |
| chains | | 2*01 | | | |

### Example 13

This example demonstrates that PBL transduced to express a TCR α chain comprising SEQ ID NO: 157 and a TCR β chain comprising SEQ ID NO: 158 are reactive against HLA-A11+/G12D 10-mer+ targets.

The two dominant α chains and the β chains of Table 17 were individually cloned into MSGV1 retroviral vectors. PBL were individually co-transduced to express one of the two pairs of the α and β chain, as shown in Table 18. Transduced PBL were screened for reactivity against (i) COS7/A11 cells transduced with a (a) G12D minigene (encoding 23-mer SEQ ID NO: 119) (COS/A11/G12D), (b) G12V minigene (encoding 23-mer SEQ ID NO: 120) (COS/A11/G12V), (c) WT KRAS minigene (encoding 23-mer SEQ ID NO: 118) (COS/A11/WT), or (d) no cells (medium only) (Table 18). IFN-γ secretion was measured.

The results are shown in Table 18. As shown in Table 18, PBL co-transduced to express murine TCR α chain TRAV4-4*01 (1) (SEQ ID NO: 157) and murine TCR β chain TRBV12-2*01 (SEQ ID NO: 158) demonstrated reactivity against HLA-A11-expressing G12D transfectant target cells.

TABLE 18

| | IFN-γ (pg/ml) | | | |
|---|---|---|---|---|
| | Cos/A11/ | Cos/A11/ | Cos/A11/ | |
| | WT | G1213 | G12V | Medium |
| TRAV4-4*01(1)/ | 31 | 34440 | 32 | 32 |
| TRBV12-2*01 | | | | |
| TRAV4-4*01(2)/ | 48 | 79 | 36 | 40 |
| TRBV12-2*01 | | | | |

PBL were transduced to express the TCR TRAV4-4*01 (1)/TRBV12-2*01 (SEQ ID NOs: 157 and 158) and screened for reactivity against COS7/A11 cells transduced with a (a) G12D minigene (encoding 23-mer SEQ ID NO: 119) (COS/A11/G12D), (b) G12V minigene (encoding 23-mer SEQ ID NO: 120) (COS/A11/G12V), (c) WT KRAS minigene (encoding 23-mer SEQ ID NO: 118) (COS/A11/WT), (d) the pancreatic tumor lines shown in Table 19 that were untransduced or transduced to express HLA-A11 and the indicated KRAS mutation. In Table 19, the KRAS mutation expressed by each pancreatic tumor cell line is indicated. IFN-γ secretion was measured.

The results are shown in Table 19. As shown in Table 19, PBL transduced with the TCR TRAV4-4*01 (1)/TRBV12-2*01 (SEQ ID NOs: 157 and 158) recognized HLA-A11+G12D+ pancreatic tumor lines.

TABLE 19

| | IFN-γ (pg/ml) |
|---|---|
| | TRAV4-4/DV*01/TRBV12-2*01 |
| Cos/A11/WT | 96 |
| Cos/A11/G12D | 45214 |
| Cos/A11/G12V | 99 |
| BxPC3/A11 (WT)* | 22 |
| MiaPaca2/A11 | 16 |
| (G12C)* | |
| SK.PC.3/A11 | 22 |
| (G12V)** | |
| T3m4/A11 (Q61H)* | 42 |
| Barr (A11+, G12R)* | 17 |
| AsPC-1/A11 (G12D)** | 7321 |
| FA6-2/A11 (G12D)** | 11287 |
| MDA-Panc-48/A11 | 238 |
| (G12D)** | |
| PANC-1 (A11+, | 114 |
| G12D)** | |
| PK.45p/A11 (G12D)** | 70 |
| BxPC3 | 16 |
| MiaPaca2 | 16 |
| SK.PC.3 | 17 |
| T3m4 | 38 |
| AsPC-1 | 16 |
| FA6-2 | 16 |
| MDA-Panc-48 | 16 |
| PK.45p | 16 |
| Medium | 16 |

*Mutation (or lack thereof, i.e., “WT”) determined by genotyping.
**Mutation determined by genotyping and mRNA expression (see Tables 13 and 20).

### Example 14

This example demonstrates the correlation between IFN-γ production and mutated KRAS expression for the TRAV4-4*01 (1)/TRBV12-2*01 murine anti-KRAS G12D TCR (SEQ ID NOs: 157 and 158).

The number of copies of KRAS G12D mRNA expressed by each of the pancreatic tumor cell lines shown in Table 20 was measured and compared to the number of copies of β-actin mRNA expressed by each cell line (Table 20). The amount of IFN-γ secreted by the PBL transduced with the TRAV4-4*01 (1)/TRBV12-2*01 murine anti-KRAS G12D TCR (SEQ ID NOs: 157 and 158) upon co-culture with each cell line is shown in Table 13. The reactivity of the TRAV4-4*01 (1)/TRBV12-2*01 murine anti-KRAS G12D TCR (SEQ ID NOs: 157 and 158) (in terms of IFN-γ secretion upon co-culture with target cells) was correlated with the number of copies of KRAS G12D mRNA.

TABLE 20

| | β-actin | Ref Total KRAS | G12D (Copy | |
|---|---|---|---|---|
| | (Copy | (Copy per 10⁶ | per 10⁶ | IFN-γ |
| | number) | β-actin) | β-actin) | (pg/ml) |
| BxPC3/A11 | 3.13 × 10⁷ | 6.22 × 10³ | 2.91 × 10¹ | 26 |
| Barr | 1.88 × 10⁷ | 7.98 × 10³ | 2.41 × 10⁻¹ | 43 |
| T3m4/A11 | 3.40 × 10⁷ | 1.56 × 10⁴ | 5.26 × 10⁻¹ | 49 |
| ASPC-1/A11 | 2.69 × 10⁷ | 1.40 × 10⁴ | 5.99 × 10³ | 7320 |
| FA6-2/A11 | 3.01 × 10⁷ | 1.1 × 10⁵ | 3.99 × 10⁴ | 31688 |
| MDA-Panc- | 4.56 × 10⁷ | 4.01 × 10³ | 1.90 × 10³ | 433 |
| 48/A11 | | | | |
| PANC-1 | 3.48 × 10⁷ | 1.39 × 10⁴ | 4.28 × 10³ | 17 |
| PK.45p/A11 | 4.04 × 10⁷ | 1.66 × 10⁴ | 2.80 × 10² | 52 |

### Example 15

This example demonstrates that the TRAV4-4/DV10*01/BV12-2*01 murine anti-KRAS G12D TCR (SEQ ID NOs: 157 and 158) has a higher affinity for pulsed target peptide as compared to the TRAV12N-3*01/BV4*01 murine anti-KRAS G12D TCR (SEQ ID NOS: 11 and 12).

PBL were transduced with either (i) T TRAV4-4/DV10*01/BV12-2*01 murine anti-KRAS G12D TCR (SEQ ID NOs: 157 and 158) or (ii) TRAV12N-3*01/BV4*01 murine anti-KRAS G12D TCR (SEQ ID NOs: 11 and 12). Transduced cells were co-cultured with Cos7/All cells pulsed with (a) G12D 10-mer peptide (SEQ ID NO: 2), (b) WT KRAS 10-mer peptide (SEQ ID NO: 30), (c) G12D 9-mer peptide (SEQ ID NO: 34), or (d) WT KRAS 9-mer peptide (SEQ ID NO: 31) at the concentrations shown in Tables 21A and 21B. IFN-γ secretion was measured.

The results are shown in Table 21A (TRAV4-4/DV10*01/BV12-2*0 (SEQ ID NOs: 157 and 158)) and Table 21B (TRAV12N-3*01/BV4*01 (SEQ ID NOs: 11 and 12)). As shown in Tables 21A-21B, T cells transduced with the TRAV4-4/DV10*01/BV12-2*0 (SEQ ID NOs: 157 and 158) recognized 10-mer at pulsed at a concentration of 1×10⁻⁹ M. Accordingly, the TRAV4-4/DV10*01/BV12-2*0 (SEQ ID NOs: 157 and 158) recognized pulsed target peptide with a higher avidity as compared to the TRAV12N-3*01/BV4*01 (SEQ ID NOs: 11 and 12) TCR.

TABLE 21A

| Peptide | IFN-γ (pg/ml) | | | |
|---|---|---|---|---|
| concentration | WT | WT | G12D | G12D |
| (10^{×}M) | 9-mer | 10-mer | 9-mer | 10-mer |
| −6 | 54 | 56 | 131 | 27407 |
| −7 | 53 | 57 | 60 | 29508 |
| −8 | 59 | 51 | 47 | 6131 |
| −9 | 54 | 51 | 53 | 2075 |
| −10 | 51 | 54 | 53 | 402 |
| −11 | 48 | 50 | 52 | 63 |
| −12 | 52 | 44 | 58 | 50 |
| −13 | 51 | 54 | 51 | 51 |

TABLE 21B

| Peptide | IFN-g (pg/ml) | | | |
|---|---|---|---|---|
| concentration | WT | WT | G12D | G12D |
| (10^{×}M) | 9-mer | 10-mer | 9-mer | 10-mer |
| −6 | 90 | 82 | 125 | 18948 |
| −7 | 96 | 77 | 86 | 11623 |
| −8 | 95 | 85 | 90 | 3852 |
| −9 | 88 | 102 | 92 | 108 |
| −10 | 95 | 88 | 95 | 212 |
| −11 | 84 | 81 | 88 | 103 |
| −12 | 105 | 76 | 91 | 93 |
| −13 | 103 | 92 | 84 | 93 |

### Example 16

This example demonstrates that the TRAV4-4/DV10*01/BV12-2*01 murine anti-KRAS G12D TCR (SEQ ID NOs: 157 and 158) has a higher affinity for G12D+ pancreatic tumor cell lines as compared to TRAV12N-3*01/BV4*01 murine anti-KRAS G12D TCR (SEQ ID NOs: 11 and 12).

PBL were transduced with either (i) TRAV4-4/DV10*01/BV12-2*01 murine anti-KRAS G12D TCR (SEQ ID NOs: 157 and 158) or (ii) TRAV12N-3*01/BV4*01 murine anti-KRAS G12D TCR (SEQ ID NOs: 11 and 12). Transduced cells were co-cultured with pancreatic cell lines that were untransduced or transduced with HLA-A11 and mutated KRAS as shown in Table 22. IFN-γ secretion was measured.

The results are shown in Table 22. As shown in Table 22, T cells transduced with the TRAV4-4/DV10*01/BV12-2*0 (SEQ ID NOs: 157 and 158) recognized G12D+ pancreatic tumor cell lines with a higher avidity as compared to the TRAV12N-3*01/BV4*01 (SEQ ID NOs: 11 and 12) TCR.

TABLE 22

| | IFN-g (pg/ml) | |
|---|---|---|
| | TRAV4-4/DV10*01/ | TRAV12N-3*01/ |
| | BV12-2*01 | BV4*01 |
| BxPC3/A11 (WT)* | 28 | 37 |
| MiaPaca2/A11 (G12C)* | 27 | 57 |
| SK.PC.3/A11 (G12V)** | 41 | 44 |
| T3m4/A11 (Q61H)* | 42 | 135 |
| Barr (A11+, G12R)* | 31 | 21 |
| AsPC-1/A11 (G12D)** | 7478 | 980 |
| FA6-2/A11 (G12D)** | 8027 | 1494 |
| MDA-Panc-48/A11 | 362 | 66 |
| (G12D)** | | |
| PANC-1 (A11+, G12D)** | 148 | 34 |
| PK.45p/A11 (G12D)** | 52 | 113 |
| AsPC-1 | 24 | 16 |
| FA6-2 | 41 | 26 |
| MDA-Panc-48 | 43 | 134 |
| PK.45p | 31 | 35 |
| Medium | 28 | 20 |

*Mutation (or lack thereof, i.e., “WT”) determined by genotyping.
**Mutation determined by genotyping and mRNA expression (see Tables 13 and 20).

### Example 17

This example demonstrates a Phase I/II study administering PBL transduced with a vector encoding the murine TCR recognizing mutated KRAS to patients with mutated KRAS-expressing cancer.

To be eligible for inclusion in the study, patients meet the normal criteria for adoptive cell therapy (ACT)/IL-2 and have the following:
an HLA-A11+, mutated KRAS-expressing tumor (as measured by immunohistochemistry);
radioiodine-refractory cancer; and
a positron emission tomography (PET) avid tumor or demonstrate tumor progression within the last 6 months.

Autologous PBL are retovirally transduced with a vector encoding the alpha and beta chains of the murine anti-mutated KRAS TCR (SEQ ID NOs: 11 and 12). The patient is treated with preparative, non-myeloablative, high-dose cyclophosphamide (Cy) and fludarabine (Flu). The patient is treated with high-dose, IL-2 every eight hours until tolerance. In Phase I, the patient is treated with a starting dose of 1×10⁸ retrovirally transduced cells. The dose is increased by half-logs, with one patient per cohort up to a dose of 1×10¹⁰ cells, followed by three patients per cohort. Phase II has a two-stage design with a targeted response rate of 20%.

### Example 18

This example demonstrates the frequency of KRAS mutations in human cancers.

The frequency (%) of KRAS mutations in various human cancers is set forth in Table 23. Table 23 also shows the frequency (%) of specific KRAS mutations among all KRAS mutations.

TABLE 23

| | Frequency | % of all KRAS mutations | | | | | | |
|---|---|---|---|---|---|---|---|---|
| Tumor | of KRAS mutation | G12A | G12D | G12R | G12C | G12S | G12V | G13D |
| Pancreatic | 70% | 2 | 51 | 12 | 3 | 2 | 30 | 1 |
| carcinoma | | | | | | | | |
| Colorectal | 36% | 7 | 34 | 1 | 9 | 5 | 24 | 19 |
| Lung | 20% | 7 | 17 | 2 | 42 | 5 | 20 | 2 |
| adenocarcinoma | | | | | | | | |
| Endometrial | 18% | 11 | 36 | 0 | 9 | 2 | 24 | 15 |
| Epithelial | 14% | 4 | 41 | 2 | 5 | 0 | 37 | 5 |
| ovarian cancer | | | | | | | | |
| Prostate | 7% | 2 | 22 | 1 | 10 | 3 | 35 | 23 |

### Example 19

This example demonstrates that a substitution of the glycine residue in the CDR3α region of the TRAV4-4/DV10*01/BV12-2*01 TCR provides enhanced anti-KRAS reactivity as compared to the wild-type TRAV4-4/DV10*01/BV12-2*01 TCR.

The glycine residue in the CDR3α region of the TRAV4-4/DV10*01/BV12-2*01 TCR was replaced with an alanine residue to provide a substituted TRAV4-4/DV10*01/BV12-2*01 TCR (CDR3alpha G112A). PBL were transduced with either (i) wild-type TRAV4-4/DV10*01/BV12-2*01 TCR (SEQ ID NOs: 157 and 158) or (ii) substituted TRAV4-4/DV10*01/BV12-2*01 TCR (SEQ ID NOs: 209 and 158). Transduced cells were co-cultured with Cos cells transduced with HLA-A11 and WT KRAS (Cos/A11/WT), Cos cells transduced with HLA-A11 and G12D KRAS (Cos/A11/G12D), pancreatic tumor cell line FA6-2 transduced with HLA-A11 (FA6-2/A11), or pancreatic tumor cell line Panc-1. Transduced cells cultured alone (medium) served as control. IFN-γ secretion (pg/ml) was measured. The results are shown in Table 24.

TABLE 24

| | | Substituted TRAV4-4/ |
|---|---|---|
| | WT TRAV4-4/ | DV10*01/BV12-2*01 |
| | DV10*01/BV12-2*01 | (CDR3alpha G112A) |
| | (SEQ ID NOs: 157 | (SEQ ID NOs: 209 |
| | and 158) | and 158) |
| Cos/A11/WT | 51 | 64 |
| COs/A11/G12D | 465 | 634 |
| FA6-2/A11 | 2628 | 3631 |
| Panc-1 | 37 | 33 |
| Medium | 48 | 37 |

As shown in Table 24, a substitution of the glycine residue in the CDR3α region of the TRAV4-4/DV10*01/BV12-2*01 TCR provided enhanced anti-KRAS reactivity as compared to the wild-type TRAV4-4/DV10*01/BV12-2*01 TCR.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising.” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of producing a human cell, or population of human cells, expressing a TCR, the method comprising:
introducing a recombinant expression vector to a human cell, or a population of human cells, wherein the recombinant expression vector comprises a nucleotide sequence encoding a T cell receptor (TCR),
wherein the TCR has antigenic specificity for a mutated epitope presented by an HLA-A11 molecule, wherein the mutated epitope (a) comprises VVVGADGVGK (SEQ ID NO: 2) or (b) consists of VVVGAVGVGK (SEQ ID NO: 33) or VVGAVGVGK (SEQ ID NO: 35), and wherein the TCR comprises:
(a) an α chain complementarity determining region (CDR) 1 comprising the amino acid sequence of SEQ ID NO: 3, an α chain CDR2 comprising the amino acid sequence of SEQ ID NO: 4, an α chain CDR3 comprising the amino acid sequence of SEQ ID NO: 5, a β chain CDR1 comprising the amino acid sequence of SEQ ID NO: 6, a β chain CDR2 comprising the amino acid sequence of SEQ ID NO: 7, and a β chain CDR3 comprising the amino acid sequence of SEQ ID NO: 8:
(b) an α chain CDR 1 comprising the amino acid sequence of SEQ ID NO: 125, an α chain CDR2 comprising the amino acid sequence of SEQ ID NO: 126, an α chain CDR3 comprising the amino acid sequence of SEQ ID NO: 127, a β chain CDR1 comprising the amino acid sequence of SEQ ID NO: 128, a β chain CDR2 comprising the amino acid sequence of SEQ ID NO: 129, and a β chain CDR3 comprising the amino acid sequence of SEQ ID NO: 130;
(c) an α chain CDR 1 comprising the amino acid sequence of SEQ ID NO: 137, an α chain CDR2 comprising the amino acid sequence of SEQ ID NO: 138, an α chain CDR3 comprising the amino acid sequence of SEQ ID NO: 139, a β chain CDR1 comprising the amino acid sequence of SEQ ID NO: 140, a β chain CDR2 comprising the amino acid sequence of SEQ ID NO: 141, and a β chain CDR3 comprising the amino acid sequence of SEQ ID NO: 142:
(d) an α chain CDR 1 comprising the amino acid sequence of SEQ ID NO: 149, an α chain CDR2 comprising the amino acid sequence of SEQ ID NO: 150, an α chain CDR3 comprising the amino acid sequence of SEQ ID NO: 151, a β chain CDR1 comprising the amino acid sequence of SEQ ID NO: 152, a β chain CDR2 comprising the amino acid sequence of SEQ ID NO: 153, and a β chain CDR3 comprising the amino acid sequence of SEQ ID NO: 154; or
(e) an α chain CDR1 comprising the amino acid sequence of SEQ ID NO: 149, an α chain CDR2 comprising the amino acid sequence of SEQ ID NO: 150, an α chain CDR3 comprising the amino acid sequence of SEQ ID NO: 207, a β chain CDR1 comprising the amino acid sequence of SEQ ID NO: 152, a β chain CDR2 comprising the amino acid sequence of SEQ ID NO: 153, and a β chain CDR3 comprising the amino acid sequence of SEQ ID NO: 154.

2. The method of claim 1, wherein the TCR comprises:
(a) an α chain variable region comprising the amino acid sequence of SEQ ID NO: 9 and a β chain variable region comprising the amino acid sequence of SEQ ID NO: 10;
(b) an α chain variable region comprising the amino acid sequence of SEQ ID NO: 131 and a β chain variable region comprising the amino acid sequence of SEQ ID NO: 132;
(c) an α chain variable region comprising the amino acid sequence of SEQ ID NO: 143 and a β chain variable region comprising the amino acid sequence of SEQ ID NO: 144;
(d) an α chain variable region comprising the amino acid sequence of SEQ ID NO: 155 and a β chain variable region comprising the amino acid sequence of SEQ ID NO: 156; or
(e) an α chain variable region comprising the amino acid sequence of SEQ ID NO: 208 and a β chain variable region comprising the amino acid sequence of SEQ ID NO: 156.

3. The method of claim 1, wherein the TCR further comprises:
(a) an α chain constant region comprising the amino acid sequence of SEQ ID NO: 13 and a β chain constant region comprising the amino acid sequence of SEQ ID NO: 14;
(b) an α chain constant region comprising the amino acid sequence of SEQ ID NO: 135 and a β chain constant region comprising the amino acid sequence of SEQ ID NO: 136;
(c) an α chain constant region comprising the amino acid sequence of SEQ ID NO: 147 and a β chain constant region comprising the amino acid sequence of SEQ ID NO: 148; or
(d) an α chain constant region comprising the amino acid sequence of SEQ ID NO: 159 and a β chain constant region comprising the amino acid sequence of SEQ ID NO: 160.

4. The method of claim 1, wherein the TCR comprises:
(a) an α chain comprising the amino acid sequence of SEQ ID NO: 11 and a β chain comprising the amino acid sequence of SEQ ID NO: 12;
(b) an α chain comprising the amino acid sequence of SEQ ID NO: 133 and a β chain comprising the amino acid sequence of SEQ ID NO: 134;
(c) an α chain comprising the amino acid sequence of SEQ ID NO: 145 and a β chain comprising the amino acid sequence of SEQ ID NO: 146;
(d) an α chain comprising the amino acid sequence of SEQ ID NO: 157 and a β chain comprising the amino acid sequence of SEQ ID NO: 158; or
(e) an α chain comprising the amino acid sequence of SEQ ID NO: 209 and a β chain comprising the amino acid sequence of SEQ ID NO: 158.

5. The method of claim 1, wherein the TCR has antigenic specificity for the mutated epitope comprising VVVGADGVGK (SEQ ID NO: 2).

6. The method of claim 1, wherein the TCR has antigenic specificity for the mutated epitope consisting of VVVGAVGVGK (SEQ ID NO: 33) or VVGAVGVGK (SEQ ID NO: 35).

7. A method of producing a human cell expressing a polypeptide, or a population of human cells expressing the polypeptide, the method comprising:
introducing a recombinant expression vector to an isolated human cell, or an isolated population of human cells,
wherein the recombinant expression vector comprises a nucleotide sequence encoding the polypeptide,
wherein the polypeptide comprises a functional portion of a TCR, and wherein the functional portion comprises:
(i) the amino acid sequences of SEQ ID NOs: 3-8;
(ii) the amino acid sequences of SEQ ID NOs: 125-130;
(iii) the amino acid sequences of SEQ ID NOs: 137-142;
(iv) the amino acid sequences of SEQ ID NOs: 149-154; or
(v) the amino acid sequences of SEQ ID NOs: 149-150, 207, and 152-154.

8. The method of claim 7, wherein the functional portion comprises:
(i) the amino acid sequence of both SEQ ID NOs: 9 and 10;
(ii) the amino acid sequence of both SEQ ID NOs: 131 and 132;
(iii) the amino acid sequence of both SEQ ID NOs: 143 and 144;
(iv) the amino acid sequence of both SEQ ID NOs: 155 and 156; or
(v) the amino acid sequence of both SEQ ID NOs: 208 and 156.

9. The method of claim 7, wherein the functional portion comprises:
(i) the amino acid sequence of both SEQ ID NOs: 11 and 12:
(ii) the amino acid sequence of both SEQ ID NOs: 133 and 134;
(iii) the amino acid sequence of both SEQ ID NOs: 145 and 146;
(iv) the amino acid sequence of both SEQ ID NOs: 157 and 158; or
(v) the amino acid sequence of both SEQ ID NOs: 209 and 158.

10. A method of producing a human cell expressing a protein, or a population of human cells expressing the protein, the method comprising:
introducing a recombinant expression vector to an isolated human cell, or an isolated population of human cells,
wherein the recombinant expression vector comprises a nucleotide sequence encoding the protein, and
wherein the protein comprises:
(a) a first polypeptide chain comprising the amino acid sequences of SEQ ID NOs: 3-5 and a second polypeptide chain comprising the amino acid sequences of SEQ ID NOs: 6-8;
(b) a first polypeptide chain comprising the amino acid sequences of SEQ ID NOs: 125-127 and a second polypeptide chain comprising the amino acid sequences of SEQ ID NOs: 128-130;
(c) a first polypeptide chain comprising the amino acid sequences of SEQ ID NOs: 137-139 and a second polypeptide chain comprising the amino acid sequences of SEQ ID NOs: 140-142;
(d) a first polypeptide chain comprising the amino acid sequences of SEQ ID NOs: 149-151 and a second polypeptide chain comprising the amino acid sequences of SEQ ID NOs: 152-154; or
(e) a first polypeptide chain comprising the amino acid sequences of SEQ ID NOs: 149, 150, and 207 and a second polypeptide chain comprising the amino acid sequences of SEQ ID NOs: 152-154.

11. The method according to claim 10, wherein:
(a) the first polypeptide chain comprises the amino acid sequence of SEQ ID NO: 9 and the second polypeptide chain comprises the amino acid sequence of SEQ ID NO: 10;
(b) the first polypeptide chain comprises the amino acid sequence of SEQ ID NO: 131 and the second polypeptide chain comprises the amino acid sequence of SEQ ID NO: 132;
(c) the first polypeptide chain comprises the amino acid sequence of SEQ ID NO: 143 and the second polypeptide chain comprises the amino acid sequence of SEQ ID NO: 144;
(d) the first polypeptide chain comprises the amino acid sequence of SEQ ID NO: 155 and the second polypeptide chain comprises the amino acid sequence of SEQ ID NO: 156; or
(e) the first polypeptide chain comprises the amino acid sequence of SEQ ID NO: 208 and the second polypeptide chain comprises the amino acid sequence of SEQ ID NO: 156.

12. The method of claim 10, wherein:
(a) the first polypeptide chain comprises the amino acid sequence of SEQ ID NO: 11 and the second polypeptide chain comprises the amino acid sequence of SEQ ID NO: 12;
(b) the first polypeptide chain comprises the amino acid sequence of SEQ ID NO: 133 and the second polypeptide chain comprises the amino acid sequence of SEQ ID NO: 134;
(c) the first polypeptide chain comprises the amino acid sequence of SEQ ID NO: 145 and the second polypeptide chain comprises the amino acid sequence of SEQ ID NO: 146;
(d) the first polypeptide chain comprises the amino acid sequence of SEQ ID NO: 157 and the second polypeptide chain comprises the amino acid sequence of SEQ ID NO: 158; or
(e) the first polypeptide chain comprises the amino acid sequence of SEQ ID NO: 209 and the second polypeptide chain comprises the amino acid sequence of SEQ ID NO: 158.

13. The method of claim 1, wherein the recombinant expression vector is a viral vector.

14. The method of claim 7, wherein the recombinant expression vector is a viral vector.

15. The method of claim 10, wherein the recombinant expression vector is a viral vector.

16. The method of claim 1, wherein the population of human cells is a population of human peripheral blood mononuclear cells.

17. The method of claim 7, wherein the population of human cells is a population of human peripheral blood mononuclear cells.

18. The method of claim 10, wherein the population of human cells is a population of human peripheral blood mononuclear cells.

19. The method of claim 1, wherein the recombinant expression vector is a retroviral vector.

20. The method of claim 7, wherein the recombinant expression vector is a retroviral vector.
